(19) United States
(12) Patent Application Publication
(10) Pub. No.: US 2007/0253896 A1
(43) Pub. Date: Nov. 1, 2007

(54) 7,9-Dihydro-Purin-8-One and Related Analogs as HSP90-Inhibitors

(75) Inventors: Jean-Yves Le Brazidec, San Diego, CA (US); Srinivas Rao Kasibhatla, San Diego, CA (US); David Hurst, Santee, CA (US); Marcus F. Boehm, San Diego, CA (US)

Correspondence Address: WILSON SONSINI GOODRICH & ROSATI, 650 PAGE MILL ROAD, PALO ALTO, CA, 94304-1050, US

(73) Assignee: CONFORMA THERAPEUTICS CORPORATION, San Diego, CA (US)

(21) Appl. No.: 11/671,352

(22) Filed: Feb. 5, 2007

Related U.S. Application Data
(60) Provisional application No. 60/771,065, filed on Feb. 7, 2006.

(51) Int. Cl. A61K 31/522; A61K 31/495; A61K 31/65; A61K 31/704; A61K 31/7052; A61K 36/24; A61K 38/20; A61K 38/21; A61K 39/395; A61K 51/00; A61P 35/00; A61P 37/00; A61P 43/00; C07D 473/28

(52) U.S. Cl. 424/1.11; 424/130.1; 424/133.1; 424/725; 424/85.2; 424/85.4; 424/94.1; 435/375; 514/152; 514/249; 514/263.3; 514/34; 514/45; 544/265

(57) ABSTRACT

The invention relates in general to 7,9-dihydro-purin-8-one and related compounds that show broad utility, e.g., in inhibiting heat shock protein 90 (HSP90) to thereby treat or prevent HSP90-mediated diseases.

### RELATED APPLICATIONS

This application claims the benefit of U.S. Provisional Application No. 60/771,065 filed Feb. 7, 2006, which is herein incorporated by reference in its entirety.

### FIELD OF THE INVENTION

The invention relates in general to 7,9-dihydro-purin-8-one and related compounds that show broad utility, e.g., in inhibiting heat shock protein 90 (HSP90) to thereby treat or prevent HSP90-mediated diseases.

### BACKGROUND OF THE INVENTION

HSP90s are ubiquitous chaperone proteins that are involved in folding, activation and assembly of a wide range of proteins, including key proteins involved in signal transduction, cell cycle control and transcriptional regulation. Researchers have reported that HSP90 chaperone proteins are associated with important signaling proteins, such as steroid hormone receptors and protein kinases, including, e.g., Raf-1, EGFR, v-Src family kinases, Cdk4, and ErbB-2 (Buchner J. TIBS 1999, 24, 136-141; Stepanova, L. et al. Genes Dev. 1996, 10, 1491-502; Dai, K. et al. J. Biol. Chem. 1996, 271, 22030-4). Studies further indicate that certain co-chaperones, e.g., HSP70, p60/Hop/Sti1, Hip, Bag1, HSP40/Hdj2/Hsj1, immunophilins, p23, and p50, may assist HSP90 in its function (see, e.g., Caplan, A. Trends in Cell Biol. 1999, 9, 262-68).

Ansamycin antibiotics, e.g., herbimycin A (HA), geldanamycin (GM), and 17-allylaminogeldanamycin (17-AAG) are thought to exert their anticancerous effects by tight binding of the N-terminus pocket of HSP90, thereby destabilizing substrates that normally interact with HSP90 (Stebbins, C. et al. Cell 1997, 89, 239-250). This pocket is highly conserved and has weak homology to the ATP-binding site of DNA gyrase (Stebbins, C. et al., supra; Grenert, J. P. et al. J. Biol. Chem. 1997, 272, 23843-50). Further, ATP and ADP have both been shown to bind this pocket with low affinity and to have weak ATPase activity (Proromou, C. et al. Cell 1997, 90, 65-75; Panaretou, B. et al. EMBO J. 1998, 17, 4829-36). In vitro and in vivo studies have demonstrated that occupancy of this N-terminal pocket by ansamycins and other HSP90 inhibitors alters HSP90 function and inhibits protein folding. At high concentrations, ansamycins and other HSP90 inhibitors have been shown to prevent binding of protein substrates to HSP90 (Scheibel, T. H. et al. Proc. Natl. Acad. Sci. USA 1999, 96, 1297-302; Schulte, T. W. et al. J. Biol. Chem. 1995, 270, 24585-8; Whitesell, L., et al. Proc. Natl. Acad. Sci. USA 1994, 91, 8324-8328). Ansamycins have also been demonstrated to inhibit the ATP-dependent release of chaperone-associated protein substrates (Schneider, C. L. et al. Proc. Natl. Acad. Sci., USA 1996, 93, 14536-41; Sepp-Lorenzino et al. J. Biol. Chem. 1995, 270, 16580-16587). In either event, the substrates are degraded by a ubiquitin-dependent process in the proteasome (Schneider, C. L., supra; Sepp-Lorenzino, L., et al. J. Biol. Chem. 1995, 270, 16580-16587; Whitesell, L. et al. Proc. Natl. Acad. Sci. USA 1994, 91, 8324-8328).

HSP90 substrate destabilization occurs in tumor and non-transformed cells alike and has been shown to be especially effective on a subset of signaling regulators, e.g., Raf (Schulte, T. W. et al. Biochem. Biophys. Res. Commun. 1997, 239, 655-9; Schulte, T. W., et al. J. Biol. Chem. 1995, 270, 24585-8), nuclear steroid receptors (Segnitz, B.; U. Gehring J. Biol. Chem. 1997, 272, 18694-18701; Smith, D. F. et al. Mol. Cell. Biol. 1995, 15, 6804-12), v-Src (Whitesell, L., et al. Proc. Natl. Acad. Sci. USA 1994, 91, 8324-8328) and certain transmembrane tyrosine kinases (Sepp-Lorenzino, L. et al. J. Biol. Chem. 1995, 270, 16580-16587) such as EGF receptor (EGFR) and HER2/Neu (Hartmann, F., et al. Int. J. Cancer 1997, 70, 221-9; Miller, P. et al. Cancer Res. 1994, 54, 2724-2730; Mimnaugh, E. G., et al. J. Biol. Chem. 1996, 271, 22796-801; Schnur, R. et al. J. Med. Chem. 1995, 38, 3806-3812), CDK4, and mutant p53. Erlichman et al. Proc. AACR 2001, 42, abstract 4474. The ansamycin-induced loss of these proteins leads to the selective disruption of certain regulatory pathways and results in growth arrest at specific phases of the cell cycle (Muise-Heimericks, R. C. et al. J. Biol. Chem. 1998, 273, 29864-72), and apoptosis, and/or differentiation of cells so treated (Vasilevskaya, A. et al. Cancer Res., 1999, 59, 3935-40). Ansamycins thus hold great promise for the treatment and/or prevention of many types of cancers and proliferative disorders, and also hold promise as traditional antibiotics. However, their relative insolubility makes them difficult to formulate and administer, and they are not easily synthesized and currently must, at least in part, be generated through fermentation. Further, the hepatic toxicity of ansamyins is dose limiting.

In addition to anti-cancer and antitumorgenic activity, HSP90 inhibitors have also been implicated in a wide variety of other utilities, including use as anti-inflammation agents, anti-infectious disease agents, agents for treating autoimmunity, agents for treating stroke, ischemia, multiple sclerosis, cardiac disorders, central nervous system related disorders and agents useful in promoting nerve regeneration (See, e.g., Rosen et al. WO 02/09696 (PCT/US01/23640); Degranco et al. WO 99/51223 (PCT/US99/07242); Gold, U.S. Pat. No. 6,210,974 B1; DeFranco et al., U.S. Pat. No. 6,174,875. Overlapping somewhat with the above, there are reports in the literature that fibrogenetic disorders including but not limited to scleroderma, polymyositis, systemic lupus, rheumatoid arthritis, liver cirrhosis, keloid formation, interstitial nephritis, and pulmonary fibrosis also may be treatable with HSP90 inhibitors. Strehlow, WO 02/02123 (PCT/US01/20578). Still further HSP90 modulation, modulators and uses thereof are reported in Application Nos. PCT/US03/04283, PCT/US02/35938, PCT/US02/16287, PCT/US02/06518, PCT/US98/09805, PCT/US00/09512, PCT/US01/09512, PCT/US01/23640, PCT/US01/46303, PCT/US01/46304, PCT/US02/06518, PCT/US02/29715, PCT/US02/35069, PCT/US02/35938, PCT/US02/39993, 60/293,246, 60/371,668, 60/335,391, 60/128,593, 60/337,919, 60/340,762, 60/359,484 and 60/331,893.

Recently, purine derivatives showing HSP90 inhibitory activity have been reported, e.g., in PCT/US02/35069; PCT/US02/36075. Purine moieties are well accepted bioisosteres for a variety of ATP-dependent molecular targets, see, JP 10025294; U.S. Pat. No. 4,748,177; U.S. Pat. No. 4,772,606; U.S. Pat. No. 6,369,092; WO 00/06573; WO 02/055521; WO 02/055082; WO 02/055083; European Patent 0178178; Eur. J. Med. Chem. 1994, 29(1), 3-9; and J. Het. Chem. 1990, 27(5), 1409. However, compounds having the desired potency, selectivity and pharmaceutical properties required for effective HSP90 inhibition in vivo have not been reported. Therefore, a need remains for additional novel and potent HSP90 inhibitors that meet the demanding biological and pharmaceutical criteria required to proceed towards human clinical trials.

### SUMMARY OF THE INVENTION

The present invention is directed towards heterocyclic compounds, in particular, 7,9-dihydro-purin-8-one and related compounds that show broad utility, e.g., by inhibiting HSP90 and treating diseases that are HSP90-dependent.

In one aspect, the invention comprises heterocyclic compounds as specified below in Formula I. Also included in the scope of the present invention are stereoisomic forms, including the individual enantiomers and diastereomers, racemic mixtures, and diasteromeric mixtures, and combinations thereof, where appropriate, as well as polymorphs, specific racemates and stereoisomers, solvates, esters, tautomers, pharmaceutically acceptable salts and prodrugs of these compounds. Stereoisomers of the compounds of the present invention may be isolated by standard resolution techniques such as, for example, fractional crystallization and chiral column chromatography.

In one embodiment, the invention provides compounds of Formula I, or polymorphs, solvates, esters, tautomers, diastereomers, enantiomers, pharmaceutically acceptable salts or prodrugs thereof, which show utility by inhibiting HSP90 and treating and preventing diseases that are HSP90-dependent. 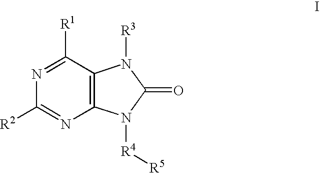
wherein:

R¹ is halogen, —OR⁸, —SR⁸, or lower alkyl;

R² is —NR⁸R¹⁰;

R³ is selected from the group consisting of hydrogen, —C(O)OH, —C(O)R⁹, —CH₂CN, —CN, alkyl, substituted alkyl, alkenyl, substituted alkenyl, alkynyl, substituted alkynyl, perhaloalkyl, substituted perhaloalkyl, alkoxyalkylene, substituted alkoxyalkylene, perhaloalkoxy, perhaloacyl, lower alkyl, lower alkenyl, lower alkynyl, lower perhaloalkyl, aryl, heteroaryl, alicyclic, heterocyclic, benzyl, substituted benzyl, phenethyl, substituted phenethyl, —(CH₂)ₘ-alicyclyl, —(CH₂)ₘ substituted alicyclyl, —(CH₂)ₘ-aryl, —(CH₂)ₘ-substituted aryl, —(CH₂)ₘ-heterocyclyl, —(CH₂)ₘ-substituted heterocyclyl, —(CH₂)ₘ-heteroaryl, —(CH₂)ₘ-substituted heteroaryl and —(CH₂)ₘ—NR⁸R¹⁰; wherein
m=1, 2 or 3;
the substituents are selected from the group comprising hydrogen, halogen, lower alkyl, lower alkenyl, lower alkynyl, —CN, —C(O)OH, —NO₂, —OR⁸, —SR⁸, —C(O)R⁹, —NR⁸R¹⁰, lower aryl, heteroaryl, alicyclyl, lower heterocyclyl, aryalkyl, heteroaralkyl, amino, alkylamino, dialkylamino, diarylalkylamino, perhaloalkyl, perhaloalkoxy, perhaloacyl, guanidyl, pyridinyl, thiophenyl, furanyl, indolyl, indazolyl, phosphonates, phosphatyl, phosphoramidyl, sulfanyl, sulfinyl, sulfonyl, sulfonamidyl, carbamyl, uryl, thiouryl and thioamidyl;

R⁴ is —C(O)—, —C(S)—, —S(O)₂—, —S(O)₂N— or —(CH₂)ₙ—, wherein n=0, 1, 2 or 3;

R⁵ is alkyl, alicyclic, heterocyclic, aryl or heteroaryl; all optionally substituted with hydrogen, halogen, lower alkyl, lower alkenyl, lower alkynyl, lower aryl, lower heteroaryl, lower heterocyclic, lower alicyclic, aralkyl, aryloxyalkylene, alkoxyalkylene, perhaloalkyl, perhaloalkyloxy, perhaloacyl, —CN, —N₃, —NO₂, —SR⁸, —OR⁸, —C(O)R⁹ or —NR⁸R¹⁰;

R⁹ is lower alkyl, lower alkenyl, lower alkynyl, lower heterocyclic, lower aryl, lower heteroaryl, —NR⁸R¹⁰ or —OR¹¹;

R⁸ is hydrogen, lower alkyl, lower alkenyl, lower alkynyl, lower heterocyclic, lower aryl or —C(O)R⁹;

R¹⁰ is hydrogen, lower alkyl, lower alkenyl, lower alkynyl, lower heterocyclic or lower aryl; or

R⁸ and R¹⁰ are taken together with the N atom to which they are attached to form a ring comprising 3-7 ring atoms, wherein, in addition to the ring N atom, optionally 1-3 of the ring atoms are heteroatoms selected from the group O, S and N;

R¹¹ is lower alkyl, lower alkenyl, lower alkynyl or lower aryl;

with the provisos that if n=0, then R⁵ cannot be heterocyclyl; and

if R¹ is Cl or OH and R² is NH₂, then R³ cannot be H or allyl.

In certain embodiments of compounds of formula I, any aryl, heteroaryl, alicyclic or heterocyclic groups are monocyclic or bicyclic. In preferred embodiments of compounds of formula I, R¹ is halogen, R² is —NHR⁸, and R⁸ is hydrogen or —C(O)R⁹. In other preferred embodiments of compounds of formula I, R¹ is chloro or bromo, R² is —NHR⁸, R⁸ is hydrogen or —C(O)R⁹; and R³ is hydrogen, lower alkyl, lower alkenyl, lower alkynyl, lower perhaloalkyl, lower aryl, lower heteroaryl or —C(O)R⁹. In yet other embodiments of compounds of formula I, R² is —NHR⁸, R⁸ is hydrogen or —C(O)R⁹; and R⁴ is —CH₂—. In further embodiments of compounds of formula I, R¹ is halogen, R² is —NH₂ and R⁴ is —CH₂—. In a preferred embodiment of compounds of formula I, R¹ is chloro or bromo, R² is —NH₂, R⁴ is —CH₂—; and R⁵ is phenyl having at least three substituents, pyridyl having at least two substituents or 1-oxy-pyridyl having at least two substituents.

Preferred compounds of formula I are given below:

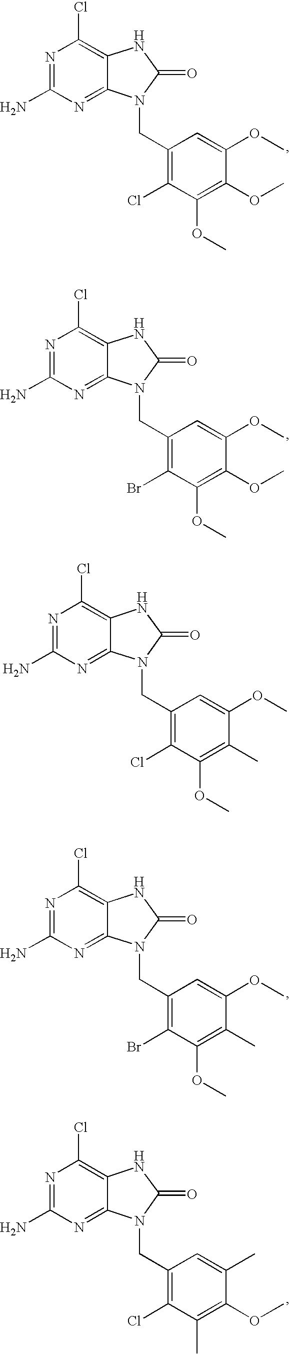

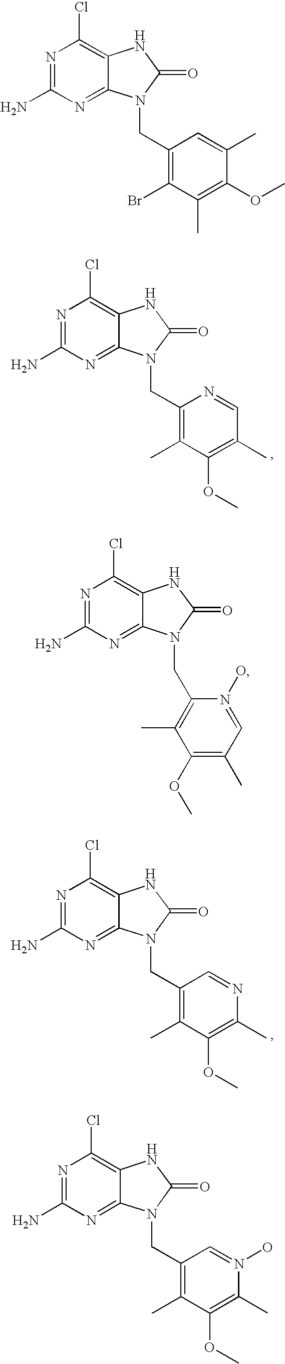

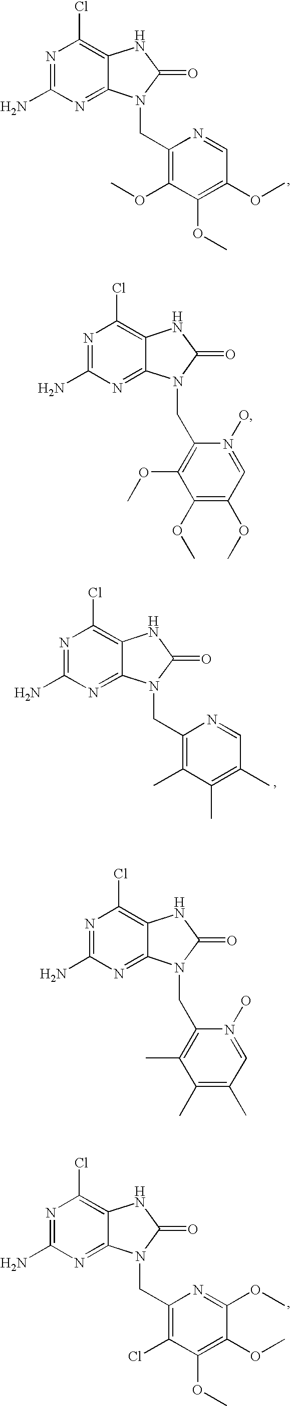

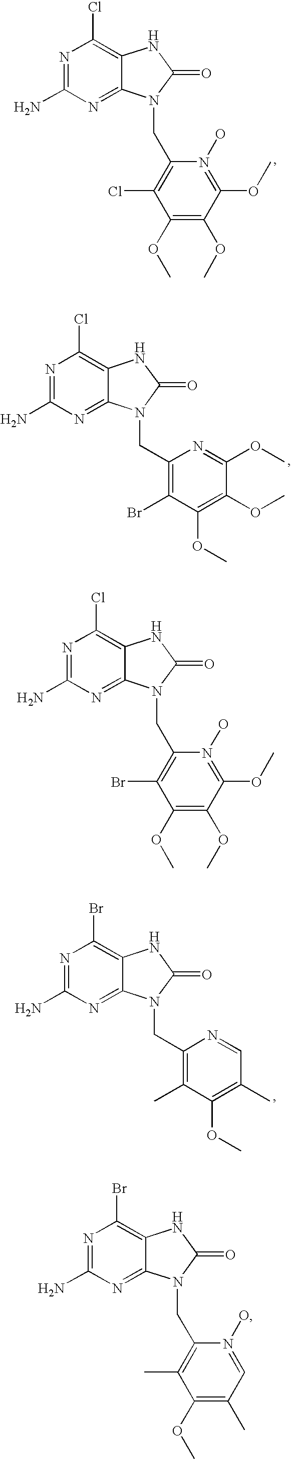

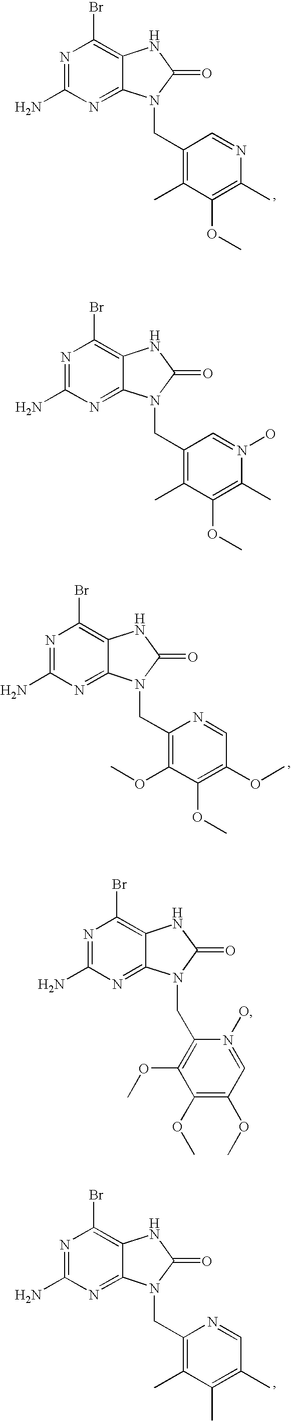

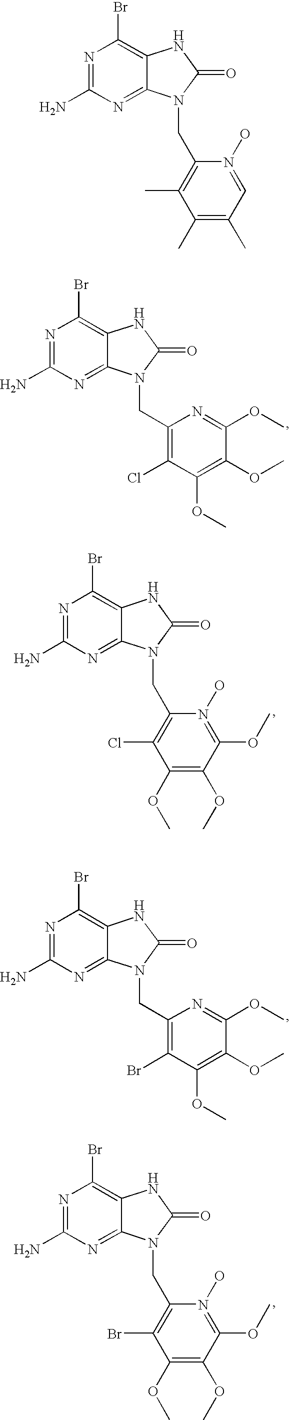

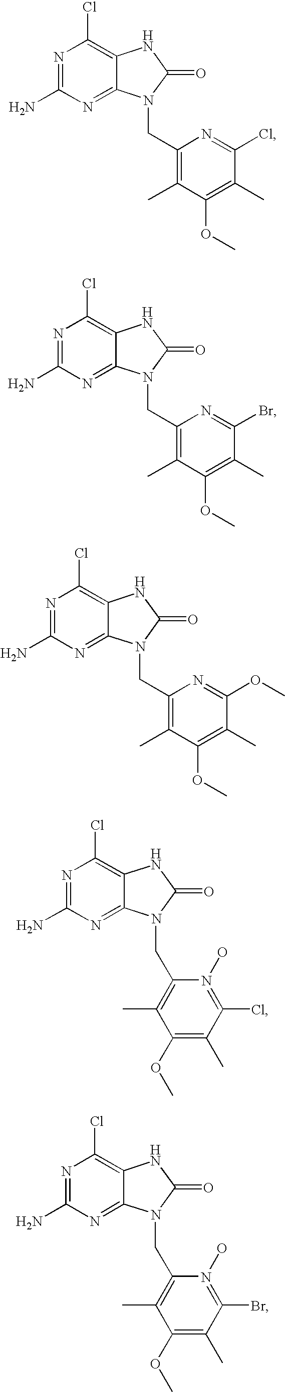

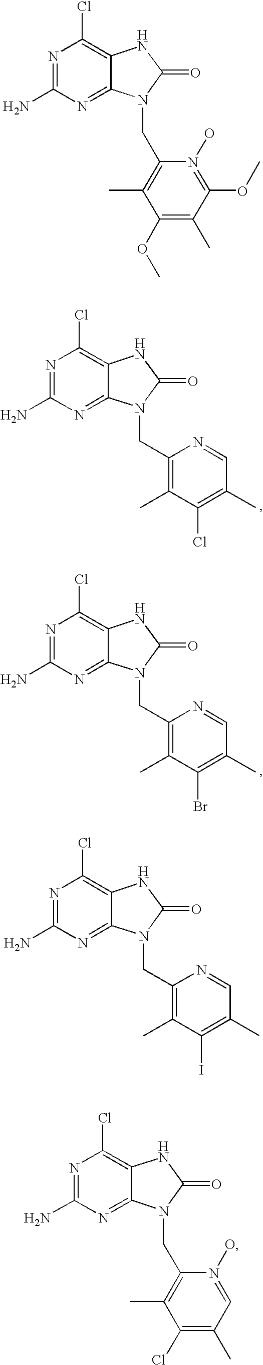

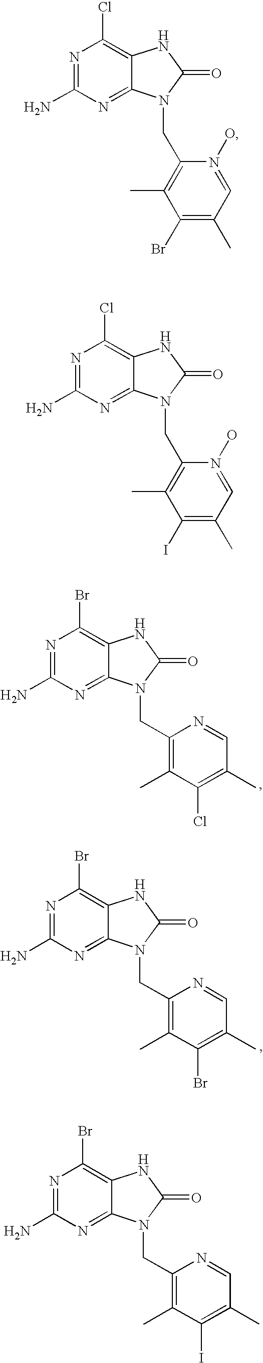

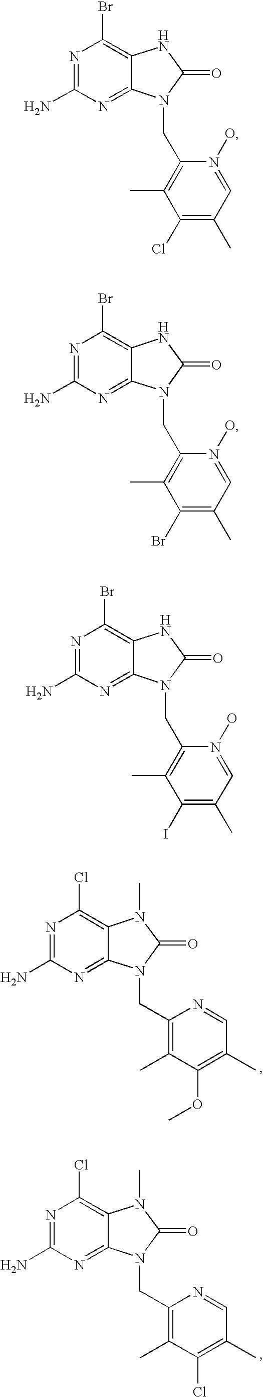

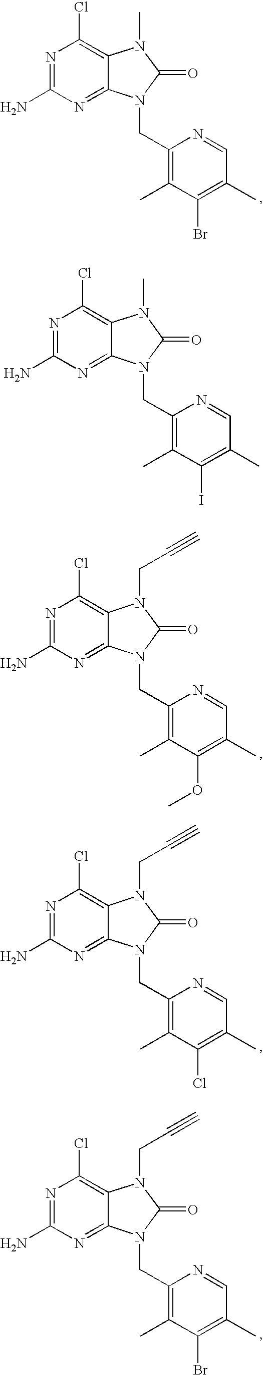

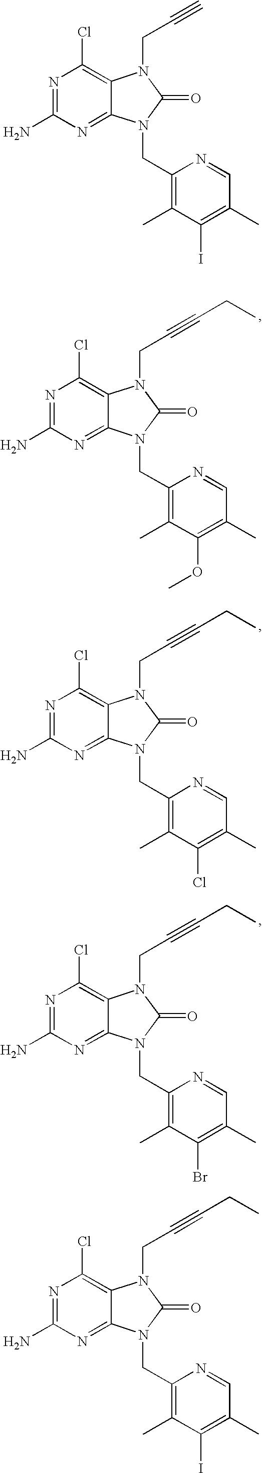

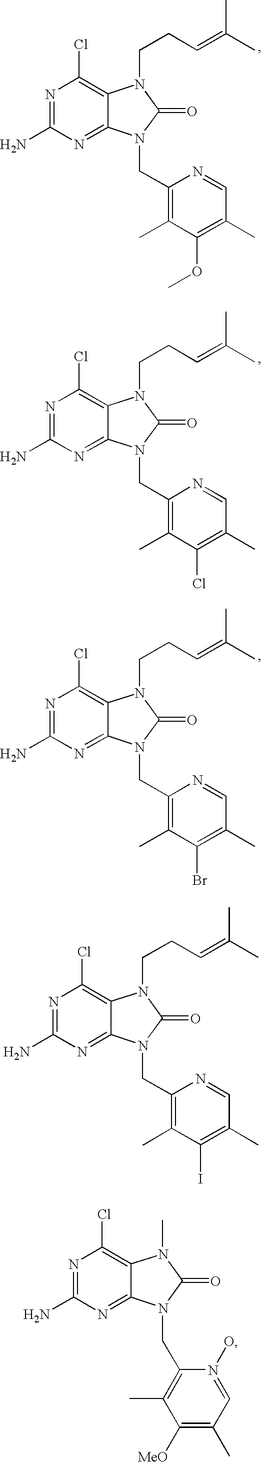

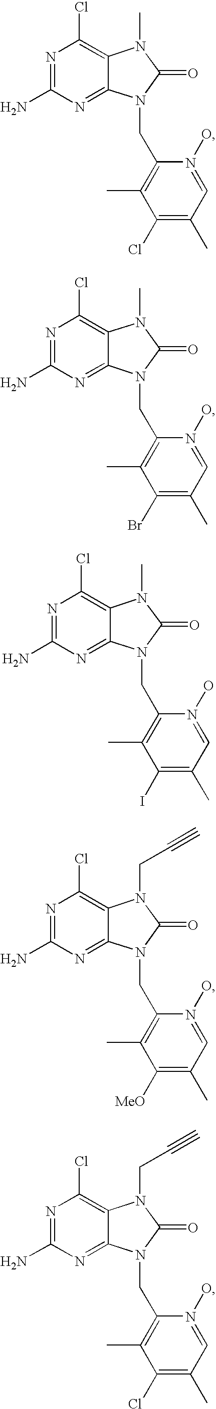

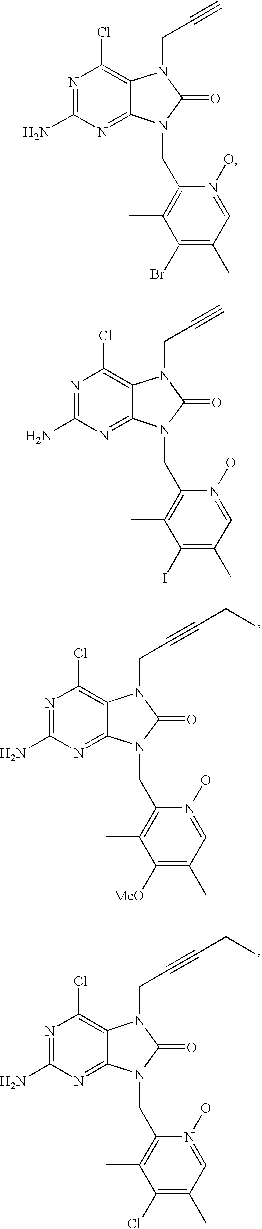

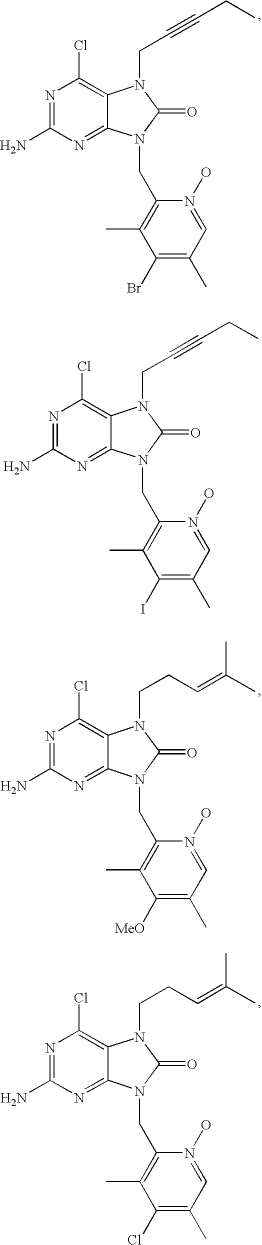

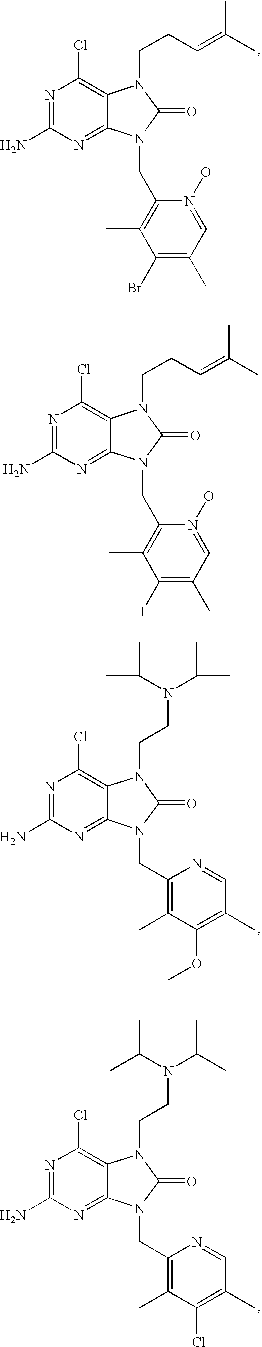

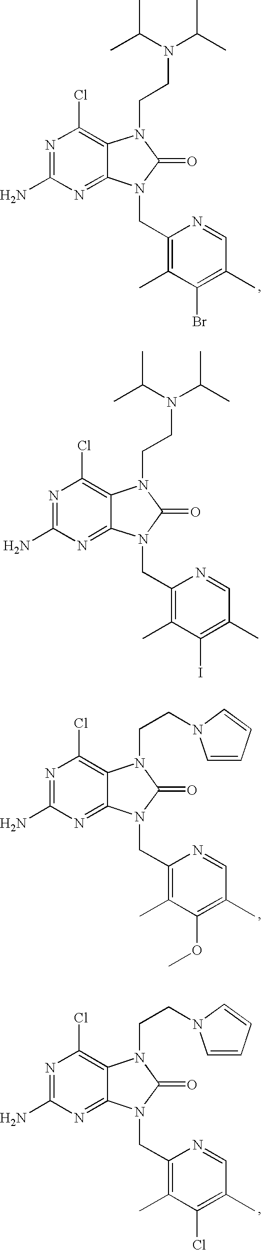

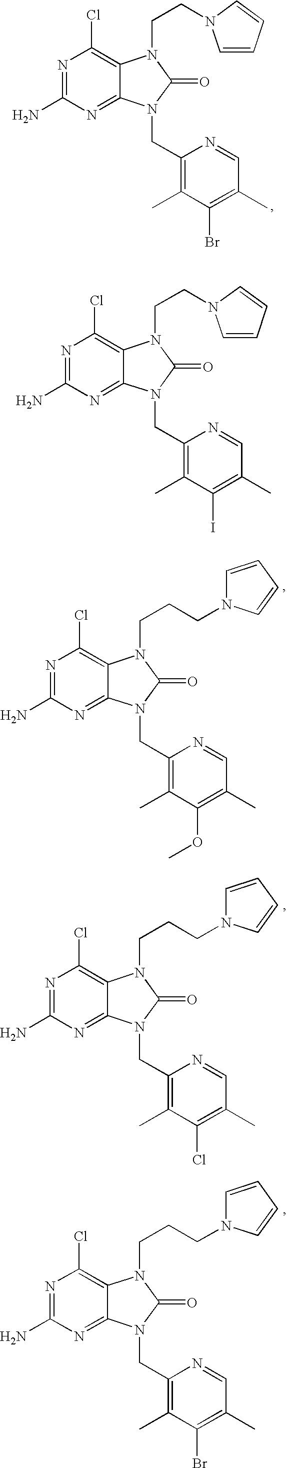

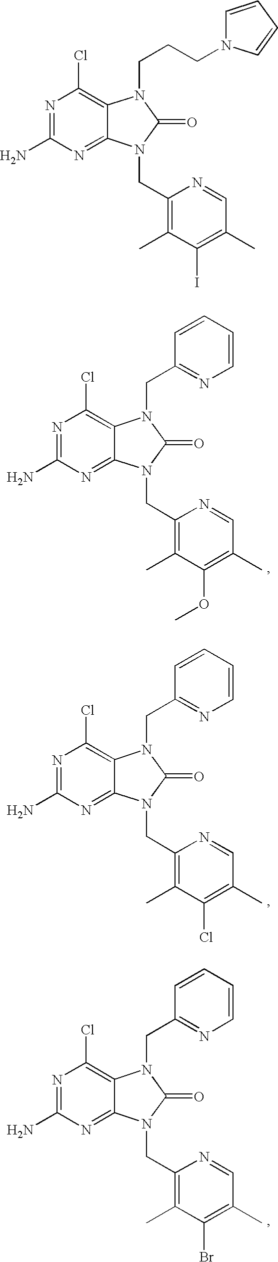

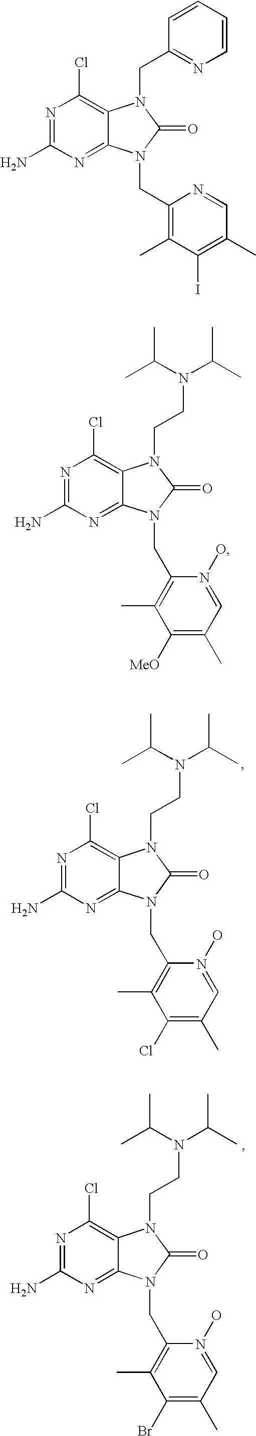

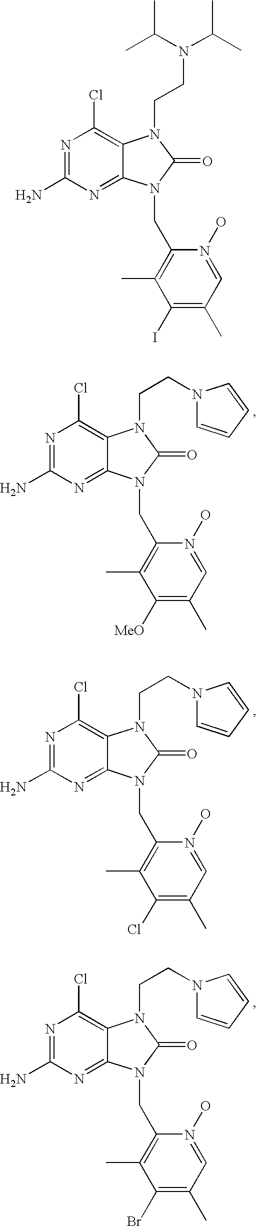

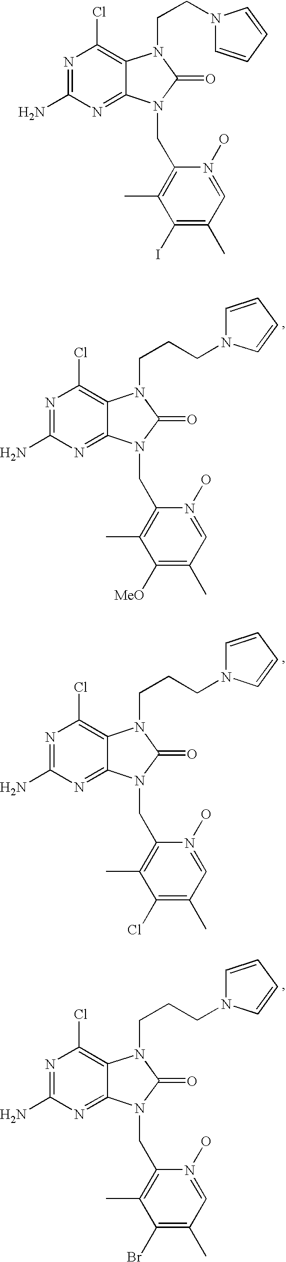

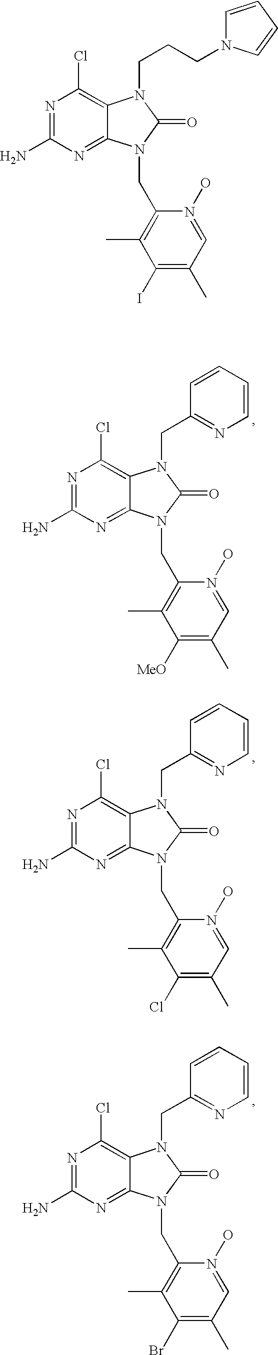

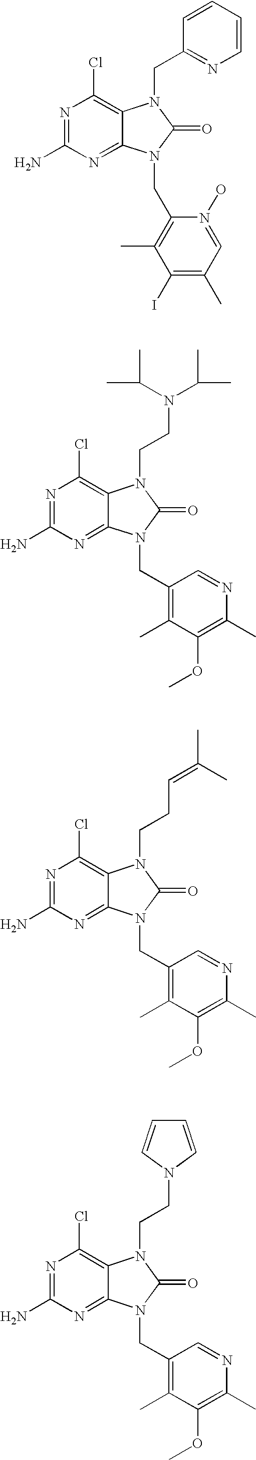

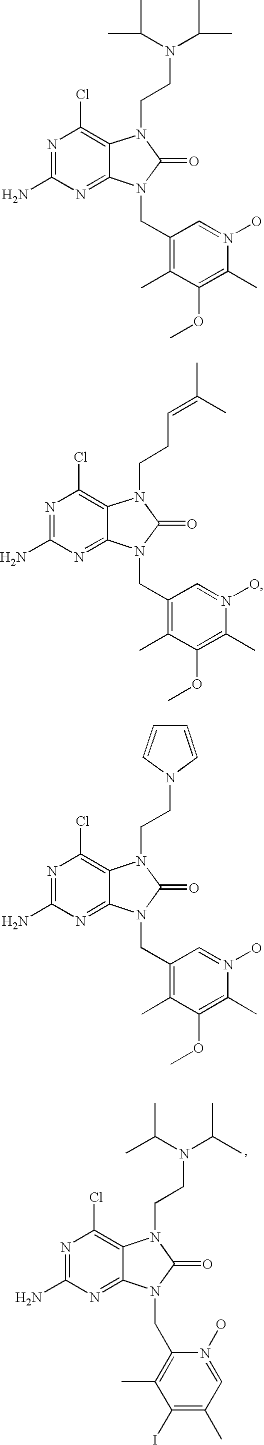

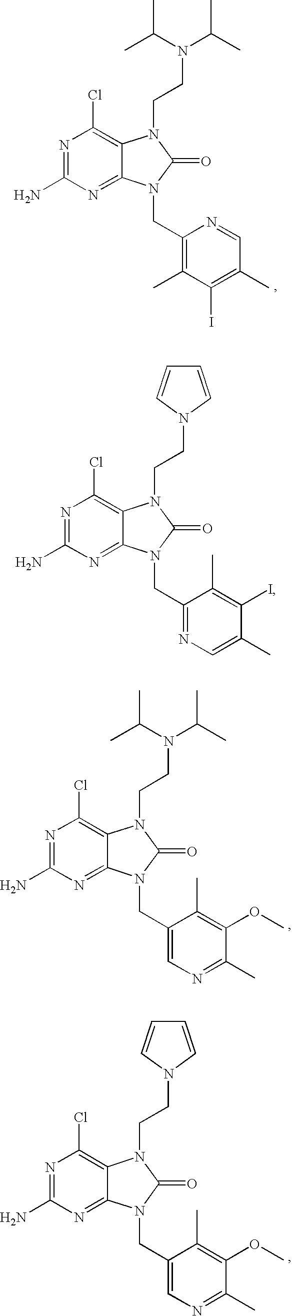

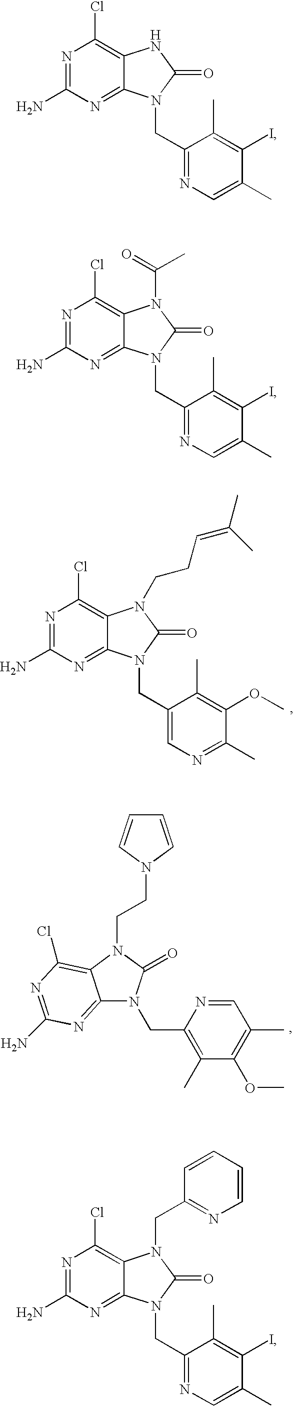

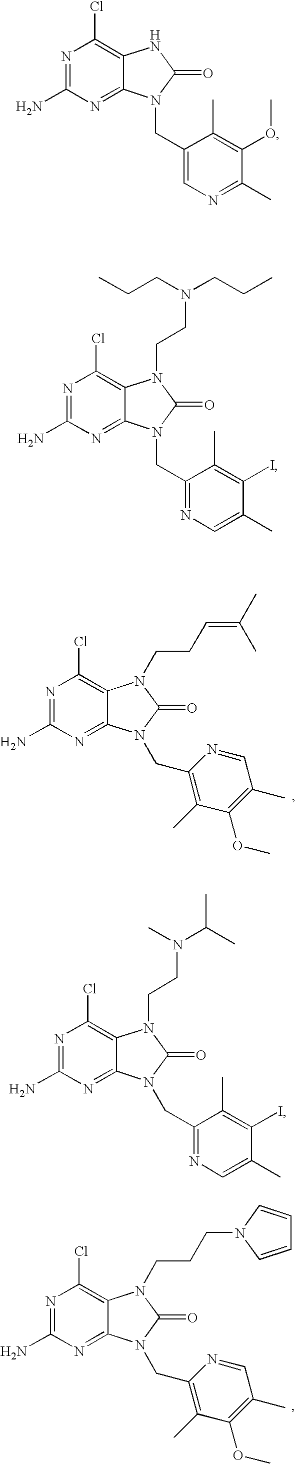

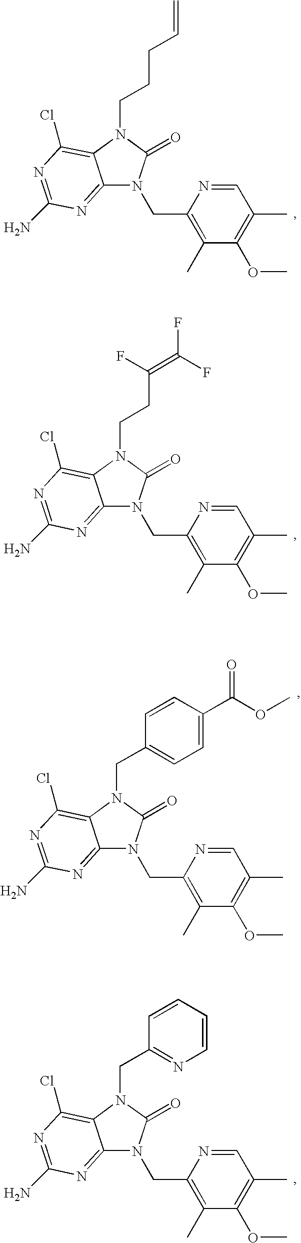

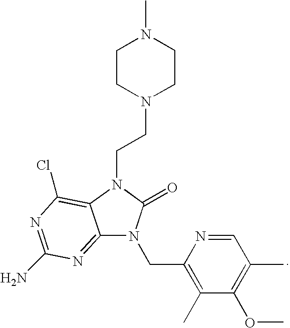

In some embodiments of the invention, the compounds of formula I may have from 0 to 25 substitutions, collectively. Another embodiment of the invention includes pharmaceutical compositions comprising compounds of formula I and one or more pharmaceutical carriers or excipients. A further embodiment of the invention includes, a complex comprising the compound of formula I and at least one other compound, preferably where the at least one other compound is an HSP90, and most preferably wherein the HSP90 is human. Another aspect of the invention involves a method of inhibiting an HSP90, comprising contacting a cell having an HSP90 with a compound of formula I. Preferably the cell is a mammalian cell, and most preferably the mammalian cell is human, and the contacting may occur in vitro, in vivo, in situ or as part of an ex vivo procedure.

In another embodiment, contacting a cell having an HSP90 with a compound of formula I is accomplished by intravenous, parenteral, oral or topical administration to a subject, and is preferably part of a therapy directed against cancer cells. In a further preferred embodiment, the cancer cells are breast cancer cells or melanoma cells.

Yet another aspect of the invention involves the use of a compound of formula I in a chemotherapy regimen, wherein said regimen may be part of a combinational therapy that makes use of one or more other agents selected from the group consisting of radioisotopes, antibodies, recombinant products, small molecules, antineoplastic agents, Herceptin, taxol, taxanes and taxane derivatives, gleevac, alkylating agents, anti-metabolites; epidophyllotoxin; an antineoplastic enzyme; a topoisomerase inhibitor; procarbazine; mitoxantrone; platinum coordination complexes; biological response modifiers/growth inhibitors; hormonal/anti-hormonal therapeutic agents and haematopoietic growth factors, antliracycline drugs, vinca drugs, mitomycins, bleomycins, cytotoxic nucleosides, tepothilones, disc odermolide, pteridine drugs, diynenes, podophyllotoxins, caminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin, podo-phyllotoxin derivatives, etoposide, etoposide phosphate or teniposide, melphalan, vinblastine, vincristine, leurosidine, vindesine, leurosine, paclitaxel, estramustine, carboplatin, cyclophosphamide, bleomycin, gemcitabine, ifosfamide, melphalan, hexamethyl melamine, thiotepa, cytarabine, edatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

In another embodiment of the invention the compounds of formula I are used for treating one or more of inflammation, infectious disease, autoimmune disease, and ischemia.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

I. Definitions

As used in the present specification, the following words and phrases are generally intended to have the meanings as set forth below, except to the extent that the context in which they are used indicates otherwise.

A “pharmaceutically acceptable derivative or prodrug” means any pharmaceutically acceptable salt, ester, salt of an ester or other derivative of a compound of this invention, which, upon administration to a recipient, is capable of providing, either directly or indirectly, a compound of this invention or a pharmaceutically active metabolite or residue thereof. Particularly favored derivatives or prodrugs are those that increase the bioavailability of the compounds of this invention when such compounds are administered to a patient (e.g., by allowing orally administered compound to be more readily absorbed into blood) or which enhance delivery of the parent compound to a biological compartment (e.g., the brain or lymphatic system).

A “pharmaceutically acceptable salt” may be prepared for any compound of the invention having a functionality capable of forming a salt, for example, an acid or base functionality. Pharmaceutically acceptable salts may be derived from organic or inorganic acids and bases. Compounds of the invention that contain one or more basic functional groups, e.g., amino or alkylamino, are capable of forming pharmaceutically acceptable salts with pharmaceutically acceptable organic and inorganic acids. These salts can be prepared in situ during the final isolation and purification of the compounds of the invention, or by separately reacting a purified compound of the invention in its free base form with a suitable organic or inorganic acid, and isolating the salt thus formed. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-napthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. See, e.g., Berge et al. “Pharmaceutical Salts”, J. Pharm. Sci. 1977, 66:1-19.

Compounds of the present invention that contain one or more acidic functional groups are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative pharmaceutically acceptable cations include alkali or alkaline earth salts such as the lithium, sodium, potassium, calcium, magnesium, and aluminum salts and the like. Illustrative examples of some of the bases that can be used include sodium hydroxide, potassium hydroxide, choline hydroxide, sodium carbonate, N+(C1-4 alkyl)4, and the like. Representative organic amines useful for the formation of base addition salts include ethylamine, diethylamine, ethylenediamine, ethanolamine, diethanolamine, piperazine and the like. This invention also envisions the quaternization of any basic nitrogen-containing groups of the compounds disclosed herein. Water or oil-soluble or dispersible products may be obtained by such quaternization. See, for example, Berge et al., supra.

Pharmaceutically acceptable prodrugs of the compounds of this invention include, but are not limited to, esters, carbonates, thiocarbonates, N-acyl derivatives, N-acyloxyalkyl derivatives, quaternary derivatives of tertiary amines, N-Mannich bases, Schiff bases, aminoacid conjugates, phosphate esters, metal salts and sulfonate esters. Suitable positions for derivatization of the compounds of the invention to create “prodrugs” include but are not limited, to, 2-amino substitution. Those of ordinary skill in the art have the knowledge and means to accomplish this without undue experimentation. Various forms of prodrugs are well known in the art. For examples of such prodrug derivatives, see “Design of Prodrugs”, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference.

The term “prodrugs” as employed herein includes, but is not limited to, the following groups and combinations of these groups:

N containing prodrugs include, but are not limited to, compounds comprising the following groups:

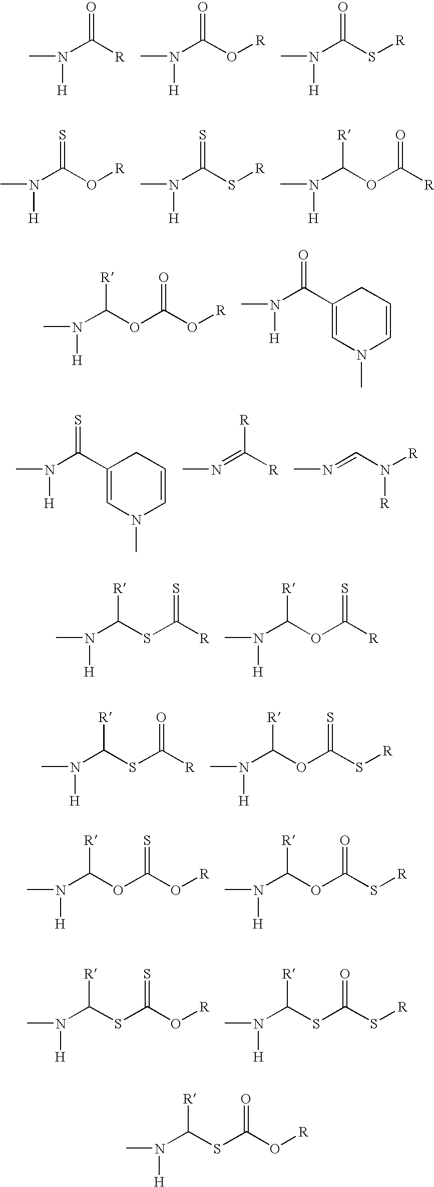

Hydroxy prodrugs include, but are not limited to acyloxyalkyl esters, alkoxycarbonyloxyalkyl esters, alkyl esters, aryl esters and disulfide containing esters.

The term “alkyl” as used herein, alone or in combination, refers to an optionally substituted straight-chain, or optionally substituted branched-chain saturated hydrocarbon monoradical having from one to about thirty carbons, more preferably one to twelve carbons. Examples of alkyl radicals include, but are not limited to methyl, ethyl, n-propyl, isopropyl, n-butyl, isobutyl, sec-butyl, tert-butyl, tert-amyl, pentyl, hexyl, heptyl, octyl and the like.

The term “cycloalkyl” as used herein, alone or in combination, refers to non-aromatic cyclic alkyl monoradicals including monocyclic, bicyclic, tricyclic, and higher multicyclic or polycyclic alkyl radicals wherein each cyclic moiety has from three to about eight carbon atoms. Examples of cycloalkyl radicals include cyclopropyl, cyclobutyl, cyclopentyl, cyclohexyl and the like.

The term “lower alkyl” as used herein, alone or in combination, refers to an alkyl containing fewer carbon atoms, e.g., one containing from one to about six carbon atoms.

The term “alkylene” as used herein, alone or in combination, refers to a diradical derived from the above-defined monoradical, alkyl. This term is exemplified by groups such as methylene (—CH₂—) and ethylene (—CH₂CH₂—).

The term “alkenyl” as used herein, alone or in combination, refers to an optionally substituted straight-chain, or optionally substituted branched-chain hydrocarbon monoradical having one or more carbon-carbon double-bonds and having from two to about thirty carbon atoms, more preferably two to about eighteen carbons. Non-limiting examples of alkenyl radicals include ethenyl (—CH═CH₂), 1-propenyl (—CH₂CH═CH₂), isopropenyl [—C(CH₃)═CH₂], butenyl, 1,3-butadienyl and the like.

The term “cycloalkenyl” as used herein, alone or in combination, refers to cyclic alkenyl radicals including monocyclic, bicyclic, tricyclic, and higher multicyclic or polycyclic alkenyl radicals wherein each cyclic moiety has from three to about eight carbon atoms.

The term “lower alkenyl” as used herein, alone or in combination, refers to an alkenyl containing fewer carbon atoms, e.g., one containing from two to about six carbon atoms.

The term “alkenylene” as used herein, alone or in combination, refers to a diradical derived from the above-defined monoradical alkenyl. A non-limiting example of an alkenylene radical includes ethenylene (—CH═CH—).

The term “alkynyl” as used herein, alone or in combination, refers to an optionally substituted straight-chain or optionally substituted branched-chain hydrocarbon monoradical having one or more carbon-carbon triple-bonds and having from two to about thirty carbon atoms, more preferably from two to about twelve carbon atoms, or from two to about six carbon atoms, as well as those having from two to about four carbon atoms. Non-limiting examples of alkynyl radicals include ethynyl, 2-propynyl, 2-butynyl, 1,3-butadienyl and the like.

The term “cycloalkanyl” as used herein, alone or in combination, refers to cyclic alkynyl radicals including monocyclic, bicyclic, tricyclic, and higher multicyclic or polycyclic alkynyl radicals wherein each cyclic moiety has from three to about eight carbon atoms.

The term “lower alkynyl” as used herein, alone or in combination, refers to an alkynyl containing fewer carbon atoms, e.g. one containing from two to about six carbon atoms.

The term “alkynylene” as used herein, alone or in combination, refers to a diradical derived from the above-defined monoradical, alkynyl. Non-limiting examples of alkynylene groups include ethynylene (—C≡C—), propargylene (—CH₂—C≡C—) and the like.

The terms “heteroalkyl”, “heteroalkenyl” and “heteroalkynyl” as used herein, alone or in combination, refers to optionally substituted alkyl, alkenyl and alkynyl structures respectively, as described above, and which have one or more skeletal chain atoms selected from an atom other than carbon, e.g., oxygen, nitrogen, sulfur, phosphorous or combinations thereof.

The term “carbon chain” as used herein, alone or in combination, refers to any alkyl, alkenyl, alkynyl, heteroalkyl, heteroalkenyl or heteroalkynyl group, which is linear, cyclic, or any combination thereof. If the chain is part of a linker and that linker comprises one or more rings as part of the core backbone, for purposes of calculating chain length, the “chain” only includes those carbon atoms that compose the bottom or top of a given ring and not both, and where the top and bottom of the ring(s) are not equivalent in length, the shorter distance shall be used in determining the chain length. If the chain contains heteroatoms as part of the backbone, those atoms are not calculated as part of the carbon chain length.

The terms “cyclic” and “membered ring” as used herein, alone or in combination, refers to any cyclic structure, including alicyclic, heterocyclic, aromatic, heteroaromatic and polycyclic fused or non-fused ring systems as described herein. The term “membered” is meant to denote the number of skeletal atoms that constitute the ring. Thus, for example, pyridine, pyran, and pyrimidine are six-membered rings and pyrrole, tetrahydrofuran, and thiophene are five-membered rings.

The term “alicyclic” as used herein, alone or in combination, refers to an optionally substituted saturated or unsaturated nonaromatic hydrocarbon ring system containing from three to about twenty ring atoms, three to about twelve carbon atoms, or from three to about ten carbon atoms. The term alicyclic includes fused, non-fused and bridged alicyclic radicals. A fused alicyclic may contain from two to four fused rings where the ring of attachment is an alicyclic ring, and the other individual rings within the fused-alicyclic radical may be alicyclic, heterocyclic, aromatic, heteroaromatic or any combination thereof. Examples of alicyclics include, without limitation, cyclopropyl, cyclopropenyl, cyclobutyl, cyclopentyl, cyclodecyl, cyclododecyl, cyclopentadienyl, indanyl, cyclooctatetraenyl, norbornyl and adamantyl ring systems.

The term “lower alicyclic” as used herein, alone or in combination, refers to an alicyclic having three to about ten skeletal ring carbons, e.g., cyclopropyl, cyclopropenyl, cyclobutyl, cyclopentyl, decalinyl, and cyclohexyl.

The term “alicyclyl” as used herein, alone or in combination, refers to a diradical derived from the above-defined monoradical, alicyclic.

The terms “heterocycle”, “heterocyclic” and “heterocyclyl” as used herein, alone or in combination, refer to optionally substituted saturated or unsaturated nonaromatic ring monoradicals containing from five to about twenty ring atoms where one or more of the ring atoms are heteroatoms such as, for example, oxygen, nitrogen, sulfur, and phosphorus. The term heterocyclic includes fused and non-fused heterocyclic ring radicals. A fused heterocyclic radical may contain from two to four fused rings where the attaching ring is a heterocyclic, and the other individual rings within the fused heterocyclic radical may be alicyclic, heterocyclic, aromatic, heteroaromatic or any combination thereof. The term heterocyclic also includes radicals having from five to about twelve skeletal ring atoms, as well as those having from five to about ten skeletal ring atoms. Example of heterocyclics include, without limitation, morpholino, piperidinyl, tetrahydrofuranyl, benzodiazepinyl, tetrahydroindazolyl, dihyroquinolinyl, and the like.

The term “lower heterocyclic” as used herein, alone or in combination, refers to a heterocyclic ring system having five to about ten skeletal ring atoms, e.g., dihydropyranyl, pyrrolidinyl, dioxolanyl, piperidinyl, piperazinyl, and the like.

The term “aromatic” refers to a cyclic or polycyclic moiety having a conjugated unsaturated (4n+2)π electron system (where n is a positive integer), sometimes referred to as a delocalized n electron system.

The term “aryl” as used herein, alone or in combination, refers to an optionally substituted aromatic hydrocarbon radical of six to about twenty ring atoms, and includes fused and non-fused aromatic rings. A fused aromatic ring radical contains from two to four fused rings where the ring of attachment is an aromatic ring, and the other individual rings within the fused ring may be alicyclic, heterocyclic, aromatic, heteroaromatic or any combination thereof. Further, the term aryl includes fused and non-fused aromatic rings containing from six to about twelve carbon atoms, as well as those containing from six to about ten carbon atoms. A non-limiting example of a single ring aryl group includes phenyl; a fused ring aryl group includes naphthyl, anthryl, azulenyl; and a non-fused bi-aryl group includes biphenyl.

The term “lower aryl” as used herein, alone or in combination, refers to an aryl group having six to about ten skeletal ring carbons, e.g., phenyl and naphthyl ring systems.

The term “arylene” as used herein, alone or in combination, refers to a diradical derived from the above-defined monoradical, aryl, and includes for example, groups such as phenylene.

The term “heteroaryl” as used herein, alone or in combination, refers to optionally substituted aromatic monoradicals containing from about five to about twenty skeletal ring atoms and where one or more of the ring atoms is a heteroatom such as, for example, oxygen, nitrogen, sulfur, selenium or phosphorus. The term heteroaryl includes optionally substituted fused and non-fused heteroaryl radicals having at least one heteroatom (e.g., quinoline, benzothiazole). A fused heteroaryl radical may contain from two to four fused rings where the ring of attachment is a heteroaromatic ring and the other individual rings within the fused ring system may be alicyclic, heterocyclic, aromatic, heteroaromatic or any combination thereof. The term heteroaryl also includes fused and non-fused heteroaryls having from five to about twelve skeletal ring atoms, as well as those having from five to about ten skeletal ring atoms. Examples of heteroaryls include, without limitation, furanyl, benzofuranyl, chromenyl, pyridyl, pyrrolyl, indolyl, quinolinyl, pyrimidyl, pyrazinyl, imidazolyl, pyrazolyl, oxazolyl, isoxazolyl, benzothiazole, benzimidazole, benzoxazole, benzothiadiazole, benzoxadiazol, benzotriazole, quinolines, isoquinolines, indoles, purinyl, indolizinyl, thienyl and the like, and their oxides, such as for example pyridyl-N-oxide.

The term “lower heteroaryl” as used herein, alone or in combination, refers to a heteroaryl having five to about ten skeletal ring atoms, e.g., pyridyl, thienyl, pyrimidyl, pyrazinyl, pyrrolyl, or furanyl.

The term “heteroarylene” as used herein, alone or in combination, refers to a diradical derived from the above-defined monoradical heteroaryl, and includes for example, groups such as pyridinyl.

The term “alkaryl” as used herein, alone or in combination, refers to the group -alkylene-aryl, wherein the terms alkylene and aryl are as defined herein, and include for example, benzyl, 2-phenylethyl and the like. Alkaryl moieties also fall within the definition of optionally substituted alkyl, e.g., as a 2-phenyl-n-pentyl moiety.

The term “aryalkyl” as used herein, alone or in combination, refers to the group -arylene-alkyl, wherein the terms arylene and alkyl are as defined herein, and include for example tolyl, xylyl and the like. Aryalkyl moieties also fall within the definition of optionally substituted aryls, e.g., as a 2-ethyl-phenyl moiety.

The term “heteroaralkyl” as used herein, alone or in combination, refers to the group -heteroarylene-alkyl, wherein the terms heteroarylene and alkyl are as defined herein, and include for example picolinoyl and the like.

The term “alkyloxy” as used herein, alone or in combination, refers to the group —O-alkyl, wherein the term alkyl is as defined herein. Non-limiting examples of alkyloxy radicals include methoxy, ethoxy, n-propoxy, isopropoxy, n-butoxy, iso-butoxy, sec-butoxy, tert-butoxy and the like.

The term “alkoxy” as used herein, alone or in combination, refers to the groups —O-alkyl, —O-alkenyl, —O-alkynyl, —O-cycloalkyl and —O-cycloalkenyl, wherein the terms alkyl, alkenyl, alkynyl, cycloalkyl and cycloalkenyl are as defined herein. Non-limiting examples of alkoxy radicals include n-propenyloxy, cyclopentyloxy and the like.

The term “aryloxy” as used herein, alone or in combination, refers to an aryl ether radical, —O-aryl, wherein the term aryl is as defined herein. A non-limiting example of an aryloxy radical is phenoxy.

The term “heteroaryloxy” as used herein, alone or in combination, refers to a heteroaryl ether radical, —O-heteroaryl, wherein the term heteroaryl is as defined herein. A non-limiting example of a heteroaryloxy radical is pyridoxy.

The term “alkoxyalkylene” as used herein, alone or in combination, refers to the group -alkylene-O-alkyl, wherein the alkylene and alkyl groups are as defined herein and may be optionally substituted. Non-limiting examples include methoxymethylene (—CH₂OCH₃), methoxyethylene (—CH₂CH₂OCH₃), n-(isopropoxy)propylene (—CH₂CH₂CH₂OCH(CH₃)₂) and the like.

The term “aryloxyalkylene” as used herein, alone or in combination, refers to the group -alkylene-O-aryl, wherein the alkylene and aryl groups are as defined herein and may be optionally substituted. A non-limiting example would be pheyloxymethylene (—CH₂OPh).

The term “alkylthio” as used herein, alone or in combination, refers to an alkyl thio radical, —S-alkyl, wherein the term alkyl is as defined herein.

The term “arylthio” as used herein, alone or in combination, refers to an aryl thio radical, —S-aryl, wherein the term aryl is as defined herein.

The term “heteroarylthio” as used herein, alone or in combination, refers to a heteroaryl thio radical, —S-heteroaryl, wherein the term heteroaryl is as defined herein.

The term “acyl” as used herein, alone or in combination, refers to an acyl radical, —C(O)R, wherein R may be, but is not limited to alkyl, alkenyl, alkynyl, alicyclic, heterocyclic, aryl, heteroaryl, arylalkyl or heteroarylalkyl, wherein the alkyl, alkenyl, alkynyl, alicyclic, heterocyclic, aryl, heteroaryl, arylalkyl or heteroarylalkyl groups may be optionally substituted.

The term “acyloxy” as used herein, alone or in combination, refers to an acyloxy radical, —OC(O)R, wherein R may be, but is not limited to H, alkyl, alkenyl, alkynyl, alicyclic, heterocyclic, aryl, heteroaryl, arylalkyl or heteroarylalkyl, wherein the alkyl, alkenyl, alkynyl, alicyclic, heterocyclic, aryl, heteroaryl, arylalkyl or heteroarylalkyl groups may be optionally substituted.

The term “carboxy ester” as used herein, alone or in combination, refers to a carboxy ester radical, —C(O)OR, wherein R may be, but is not limited to alkyl, aryl or arylalkyl, wherein the alkyl, aryl and arylalkyl groups may be optionally substituted.

The term “carboxamido” as used herein, alone or in combination, refers to a carboxamido radical, —NR′—C(O)R, wherein each R and R′ are independently selected from the group consisting of H, alkyl, alicyclic, heterocyclic, aryl, heteroaryl, arylalkyl and heteroarylalkyl, wherein the alkyl, alicyclic, heterocyclic, aryl, heteroaryl, arylalkyl and heteroarylalkyl groups may be optionally substituted.

The term “aminoacyl” as used herein, alone or in combination, refers to an aminoacyl radical, —C(O)—NRR′ wherein each R and R′ are independently selected from the group consisting of H, alkyl, alicyclic, heterocyclic, aryl, heteroaryl, arylalkyl and heteroarylalkyl, wherein the alkyl, alicyclic, heterocyclic, aryl, heteroaryl, arylalkyl and heteroarylalkyl groups may be optionally substituted.

The term “amide” as used herein encompasses both the carboxamido and the aminoacyl groups.

The term “oxo” as used herein, alone or in combination, refers to ═O.

The term “carbonyl” as used herein, alone or in combination, refers to the di-radical “—C(═O)-”, which may also be written as “—C(O)-”.

The term “carboxy” or “carboxyl” as used herein, alone or in combination, refers to the moiety “—C(O)OH”, which may also be written as “—COOH”.

The term “halogen” or “halo” as used herein refers to fluoro, chloro, bromo and iodo.

The terms “haloalkyl”, “haloalkenyl”, “haloalkynyl” and “haloalkoxy” as used herein, alone or in combination, refer to alkyl, alkenyl, alkynyl and alkoxy groups respectively, as defined herein, that are substituted with one or more fluorines, chlorines, bromines or iodines, or combinations thereof. Examples include, but are not limited to fluoromethyl and bromoethyl.

The term “perhalo” as used herein, alone or in combination, refers to groups in which all of the H atoms are replaced by fluorines, chlorines, bromines, iodines, or combinations thereof. Thus, as a non-limiting example, the term “perhaloalkyl” refers to an alkyl group, as defined herein, in which all of the H atoms have been replaced by fluorines, chlorines, bromines or iodines, or combinations thereof. A non-limiting example of a perhaloalkyl group is trifluoromethyl.

The term “amino” as used herein, alone or in combination, refers to the group —NH₂.

The term “alkylamino” as used herein, alone or in combination, refers to the group —NHR wherein R is an alkyl group as defined herein.

The term “dialkylamino” as used herein, alone or in combination, refers to the group —NRR′ wherein R and R′ are alkyl groups as defined herein.

The term “diarylalkylamino” as used herein, alone or in combination, refers to the group —N(-alkylene-aryl)₂ wherein the terms alkylene and aryl are as defined herein.

The term “aminoalkyl” as used herein, alone or in combination, refers to the group -alkylene-NH₂, wherein alkylene is as defined herein.

The terms “sulfide” and “thioether” as used herein, alone or in combination, refer to a sulfur atom covalently linked to two atoms, wherein the formal oxidation state of said sulfur is (II).

The term “sulfanyl” as used herein, alone or in combination, refers to the groups —S—R, wherein R may be, but is not limited to alkyl, alkenyl, alkynyl, aryl, alicyclic, heterocyclic, aryl, heteroaryl, arylalkyl and heteroarylalkyl, wherein the alkyl, alkenyl, alkynyl, aryl, alicyclic, heterocyclic, aryl, heteroaryl, arylalkyl and heteroarylalkyl groups may be optionally substituted. Non-limiting examples of sulfanyl groups include methylsulfanyl (—SCH₃) and iso-propylsulfanyl (—SCH(CH₃)₂) and the like.

The term “sulfoxide” as used herein, alone or in combination, refers to a sulfur atom covalently linked to three atoms, at least one of which is an oxygen atom, —S(O)—, wherein the formal oxidation state of said sulfur atom is (IV).

The term “sulfinyl” as used herein, alone or in combination, refers to the groups —S(O)—R, wherein R may be, but is not limited to alkyl, alkenyl, alkynyl, aryl, alicyclic, heterocyclic, aryl, heteroaryl, arylalkyl and heteroarylalkyl, wherein the alkyl, alkenyl, alkynyl, aryl, alicyclic, heterocyclic, aryl, heteroaryl, arylalkyl and heteroarylalkyl groups may be optionally substituted. A non-limiting example of a sulfinyl group includes methylsulfinyl (—S(O)CH₃) and the like.

The term “sulfone” as used herein, alone or in combination, refers to a sulfur atom covalently linked to four atoms, at least two of which are oxygen atoms, —S(O)₂—, wherein the formal oxidation state of said sulfur atom is (VI).

The term “sulfonyl” as used herein, alone or in combination, refers to the groups —S(O₂)—R, wherein R may be, but is not limited to alkyl, alkenyl, alkynyl, aryl, alicyclic, heterocyclic, aryl, heteroaryl, arylalkyl and heteroarylalkyl, wherein the alkyl, alkenyl, alkynyl, aryl, alicyclic, heterocyclic, aryl, heteroaryl, arylalkyl and heteroarylalkyl groups may be optionally substituted. A non-limiting example of a sulfonyl group includes methylsulfonyl (—S(O₂)CH₃) and the like.

The term “sulphonamide” as used herein, alone or in combination, refers to a sulfur atom covalently linked to four atoms, two of which are oxygen atoms and one of which is a nitrogen atom, —S(O)₂NH—, wherein the formal oxidation state of said sulfur atom is (VI).

The term “phosphite” as used herein, alone or in combination, refers to a phosphorus atom covalently linked to three carbon atoms, wherein the formal oxidation state of said phosphorus is (III).

The term “phosphonate” as used herein, alone or in combination, refers to a phosphorus atom covalently linked to four atoms, three of which are oxygen and one of which is carbon wherein the formal oxidation state of said phosphorus is (V).

The term “phosphate” as used herein, alone or in combination, refers to a phosphorus atom covalently linked to four oxygen atoms, wherein the formal oxidation state of said phosphorus is (V).

The term “phosphoramide” as used herein, alone or in combination, refers to a phosphorus atom covalently linked to four atoms, three of which are nitrogen and one of which is oxygen wherein the formal oxidation state of said phosphorus is (V).

The terms defined above are intended, where applicable, to include their optionally substituted derivatives.

The terms “optional” or “optionally” as used herein mean that the subsequently described event or circumstance may or may not occur, but need not occur, and that the description includes instances where the event or circumstance occurs and instances in which it does not. For example, “optionally substituted alkyl” means either “alkyl” or “substituted alkyl,” as defined herein. It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, the substituents described for R¹ to R¹¹ should be generally understood as having a maximum molecular weight of about 1,000 daltons, and more typically, up to about 500 Daltons.

The terms “substituents” or “substituted” as used herein, alone or in combination, refer to groups which may be used to replace another group on a molecule. Such groups may include, without limitation, one or more of the following independently selected groups, or designated subsets thereof: —CN, —NO₂, —N₃, —SH, —OH, —C(O)CH₃, —SO₂, ═S, —COOH, halo, carboxy, lower alkyl, lower alkenyl, lower alkynyl, lower alicyclic, lower heterocyclyl, lower aryl, lower heteroaryl, lower arylalkyl, lower heteroarylalkyl, lower alkoxy, lower aryloxy, lower cycloalkyl, lower cycloalkenyl, lower alkoxyalkyl, lower alkoxyaryl, lower aryloxyalkyl, lower oxyaryl, lower alkylsulfinyl, lower alkylsulfonyl, esters, amido, nitroalkyl, carbonyl, amino, alkylamino, dialkylamino, diarylalkylamino, alkylthio, arylthio, heteroarylthio, oxo, oxa, acyl, acyloxy, carboxyesters, carboxamido, perhaloalkyl, perhaloalkoxy, perhaloacyl, guanidinyl, pyridinyl, thiophenyl, furanyl, indolyl, indazolyl, phosphonates, phosphonic acid, phosphates, phosphoramidyl, sulfonates, sulfones, sulfates, sulfonamidyl, carbamates, uryl, thiouryl, thioamidyl and thioalkyl, all of which may be further optionally substituted.

Where substituent groups are specified by their conventional chemical formulas, written from left to right, they equally encompass the chemically identical substituents that would result from writing the structure from right to left; for example, —CH₂O— is equivalent to —OCH₂—.

The term “optionally substituted” as used herein, refers to groups that are substituted or un-substituted. An optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CF₃).

The term “pyridine-1-oxy” as used herein also refers to “pyridine-N-oxy”.

The term “catalytic group” as used herein refers to a chemical functional group that assists catalysis by acting as a general acid, Brønsted acid, general base, Brønsted base, nucleophile, or any other means by which the activation barrier to reaction is lowered.

Some of the compounds of the present invention may contain one or more chiral centers and therefore may exist in enantiomeric and diastereomeric forms. The scope of the present invention is intended to cover all isomers per se, as well as mixtures of cis and trans isomers, mixtures of diastereomers and racemic mixtures of enantiomers (optical isomers) as well. Further, it is possible using well known techniques to separate the various forms, and some embodiments of the invention may feature purified or enriched species of a given enantiomer or diastereomer.

A “pharmacological composition” refers to a mixture of one or more of the compounds described herein, or pharmaceutically acceptable salts thereof, with other chemical components, such as pharmaceutically acceptable carriers and/or excipients. The purpose of a pharmacological composition is to facilitate administration of a compound to an organism.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. A physiologically acceptable carrier should not cause significant irritation to an organism and does not abrogate the biological activity and properties of the administered compound.

An “excipient” refers to an inert substance added to a pharmacological composition to further facilitate administration of a compound. Examples of excipients include but are not limited to calcium carbonate, calcium phosphate, various sugars and types of starch, cellulose derivatives, gelatin, vegetable oils and polyethylene glycols.

A “pharmaceutically effective amount” means an amount which is capable of providing a therapeutic and/or prophylactic effect. The specific dose of compound administered according to this invention to obtain therapeutic and/or prophylactic effect will, of course, be determined by the particular circumstances surrounding the case, including, for example, the specific compound administered, the route of administration, the condition being treated, and the individual being treated. A typical daily dose (administered in single or divided doses) will contain a dosage level of from about 0.01 mg/kg to about 50-100 mg/kg of body weight of an active compound of the invention. Preferred daily doses generally will be from about 0.05 mg/kg to about 20 mg/kg and ideally from about 0.1 mg/kg to about 10 mg/kg. Factors such as clearance rate, half-life and maximum tolerated dose (MTD) have yet to be determined but one of ordinary skill in the art can determine these using standard procedures.

In some method embodiments, the preferred therapeutic effect is the inhibition, to some extent, of the growth of cells characteristic of a proliferative disorder, e.g., breast cancer. A therapeutic effect will also normally, but need not, relieve to some extent one or more of the symptoms other than cell growth or size of cell mass. A therapeutic effect may include, for example, one or more of 1) a reduction in the number of cells; 2) a reduction in cell size; 3) inhibition (i.e., slowing to some extent, preferably stopping) of cell infiltration into peripheral organs, e.g., in the instance of cancer metastasis; 3) inhibition (i.e., slowing to some extent, preferably stopping) of tumor metastasis; 4) inhibition, to some extent, of cell growth; and/or 5) relieving to some extent one or more of the symptoms associated with the disorder.

As used herein, the term IC50 refers to an amount, concentration or dosage of a particular test compound that achieves a 50% inhibition of a maximal response in an assay that measures such response. In some method embodiments of the invention, the “IC50” value of a compound of the invention can be greater for normal cells than for cells exhibiting a proliferative disorder, e.g., breast cancer cells. The value depends on the assay used.

By a “standard” is meant a positive or negative control. A negative control in the context of HER2 expression levels is, e.g., a sample possessing an amount of HER2 protein that correlates with a normal cell. A negative control may also include a sample that contains no HER2 protein. By contrast, a positive control does contain HER2 protein, preferably of an amount that correlates with overexpression as found in proliferative disorders, e.g., breast cancers. The controls may be from cell or tissue samples, or else contain purified ligand (or absent ligand), immobilized or otherwise. In some embodiments, one or more of the controls may be in the form of a diagnostic “dipstick.”

By “selectively targeting” is meant affecting one type of cell to a greater extent than another, e.g., in the case of cells with high as opposed to relatively low or normal HER2 levels.

II. Compounds of the Invention

Compounds of the invention and their polymorphs, solvates, esters, tautomers, diastereomers, enantiomers, pharmaceutically acceptable salts or prodrugs show utility for inhibiting HSP90 and treating and preventing diseases that are HSP90-dependent.

In one embodiment of the invention, the compounds are represented by Formula I: 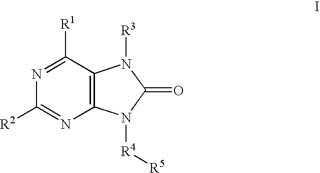
wherein:

R¹ is halogen, —OR⁸, —SR⁸, or lower alkyl;

R² is —NR⁸R¹⁰;

R³ is selected from the group consisting of hydrogen, —C(O)OH, —C(O)R⁹, —CH₂CN, —CN, alkyl, substituted alkyl, alkenyl, substituted alkenyl, alkynyl, substituted alkynyl, perhaloalkyl, substituted perhaloalkyl, alkoxyalkylene, substituted alkoxyalkylene, perhaloalkoxy, perhaloacyl, lower alkyl, lower alkenyl, lower alkynyl, lower perhaloalkyl, aryl, heteroaryl, alicyclic, heterocyclic, benzyl, substituted benzyl, phenethyl, substituted phenethyl, —(CH₂)ₘ-alicyclyl, —(CH₂)ₘ-substituted alicyclyl, —(CH₂)ₘ-aryl, —(CH₂)ₘ-substituted aryl, —(CH₂)ₘ-heterocyclyl, —(CH₂)ₘ-substituted heterocyclyl, —(CH₂)ₘ-heteroaryl, —(CH₂)ₘ-substituted heteroaryl and —(CH₂)ₘ—NR⁸R¹⁰; wherein
m=1, 2 or 3;
the substituents are selected from the group comprising hydrogen, halogen, lower alkyl, lower alkenyl, lower alkynyl, —CN, —C(O)OH, —NO₂, —OR⁸, —SR⁸, —C(O)R⁹, —NR⁸R¹⁰, lower aryl, heteroaryl, alicyclyl, lower heterocyclyl, araalkyl, heteroaraalkyl, amino, alkylamino, dialkylamino, diarylalkylamino, perhaloalkyl, perhaloalkoxy, perhaloacyl, guanidyl, pyridinyl, thiophenyl, furanyl, indolyl, indazolyl, phosphonates, phosphatyl, phosphoramidyl, sulfanyl, sulfinyl, sulfonyl, sulfonamidyl, carbamyl, uryl, thiouryl and thioamidyl;

R⁴ is —C(O)—, —C(S)—, —S(O)₂—, —S(O)₂N— or —(CH₂)ₙ—, wherein n=0, 1, 2 or 3;

R⁵ is alkyl, alicyclic, heterocyclic, aryl or heteroaryl; all optionally substituted with hydrogen, halogen, lower alkyl, lower alkenyl, lower alkynyl, lower aryl, lower heteroaryl, lower heterocyclic, lower alicyclic, aralkyl, aryloxyalkylene, alkoxyalkylene, perhaloalkyl, perhaloalkyloxy, perhaloacyl, —CN, —N₃, —NO₂, —SR⁸, —OR⁸, —C(O)R⁹ or —NR⁸R¹⁰;

R⁹ is lower alkyl, lower alkenyl, lower alkynyl, lower heterocyclic, lower aryl, lower heteroaryl, —NR⁸R¹⁰ or —OR¹¹;

R⁸ is hydrogen, lower alkyl, lower alkenyl, lower alkynyl, lower heterocyclic, lower aryl or —C(O)R⁹;

R¹⁰ is hydrogen, lower alkyl, lower alkenyl, lower alkynyl, lower heterocyclic or lower aryl; or

R⁸ and R¹⁰ are taken together with the N atom to which they are attached to form a ring comprising 3-7 ring atoms, wherein, in addition to the ring N atom, optionally 1-3 of the ring atoms are heteroatoms selected from the group O, S and N;

R¹¹ is lower alkyl, lower alkenyl, lower alkynyl or lower aryl;

with the provisos that if n=0, then R⁵ cannot be heterocyclyl; and

if R¹ is Cl or OH and R² is NH₂, then R³ cannot be H or allyl.

In one embodiment, the compound, tautomer, pharmaceutically acceptable salt thereof, or prodrug thereof of Formula I, R¹ is selected from halogen, hydroxyl, lower alkoxy, lower thioalkyl and C₁₋₄ alkyl; and R² is —NH₂.

In another embodiment, R⁴ is —(CH₂)n-, wherein n=0-3.

In another embodiment, R¹ is selected from halogen, hydroxyl, lower alkoxy, lower thioalkyl or C₁₋₄ alkyl; optionally wherein R² is NH₂.

In another embodiment, R⁴ is —(CH₂)n-, wherein n=0-3.

In another embodiment, R⁴ is —(CH₂)n-, wherein n=0-3, R¹ is selected from halogen, hydroxyl, lower alkoxy, lower thioalkyl, and C₁₋₄ alkyl, and R² is optionally NH₂.

In another embodiment, R¹ is halogen, hydroxyl, lower alkoxy, lower thioalkyl, or C₁₋₄ alkyl; and R² is optionally NH₂, R⁴ is —(CH₂)—, and R⁵ is phenyl, benzyl, or pyridyl, all optionally substituted with H, halogen, lower alkyl, —SR⁸, —OR⁸ (or cyclic ethers such as methylenedioxy), —CN, —C0₂R⁹, —NO₂, or —NR⁸R¹⁰; R⁸ is hydrogen, lower alkyl, lower aryl or —(CO)R⁹; R⁹ is lower alkyl, lower aryl, lower heteroaryl, —NR⁸R¹⁰ or —OR¹¹; R¹¹ is lower alkyl or lower aryl; and R¹⁰ is hydrogen or lower alkyl.

In another embodiment R¹ is halogen, R² is —NH₂, R⁴ is —CH₂—, R⁶ is H or halogen, and R⁵ is phenyl optionally substituted with H, halogen, C₁₋₄ alkyl, C₁₋₄ alkoxy, C₁₋₄ alkylthio, perhaloalkyl, perhaloalkyloxy, —CN, —NO₂, —NH₂ or —CO₂R¹¹.

In another embodiment, R¹ is halogen, R² is —NH₂, R⁴ is —CH₂—, R⁶ is H, and R⁵ is 2-halo-3,5-dimethoxyphenyl optionally substituted with H, halogen, C₁₋₄ alkyl, C₁₋₄ alkoxy, C₁₋₄ alkylthio, perhaloalkyl, perhaloalkyloxy, —CN, —NO₂, —NH₂, or —CO₂R¹¹ at the para (4-) position.

In another embodiment, R¹ is chloro, R² is —NH₂, R⁴ is —CH₂—, R⁶ is H and R⁵ is 2-chloro-3,4,5-trimethoxyphenyl.

In another embodiment, R¹ is chloro, R² is —NH₂, R⁴ is —CH₂—, R⁶ is H and R⁵ is 2-bromo-3,4,5-trimethoxyphenyl. In other embodiments, R⁵ is selected from 2-iodo-3,4,5-trimethoxyphenyl, 2-fluoro-3,4,5-trimethoxyphenyl, and 2-bromo-3,4,5-trimethoxyphenyl.

Any of the foregoing embodiments can be combined where feasible and appropriate.

TABLE 1
Exemplary Compounds based on Formula I

| | |
|---|---|
| | I |
| 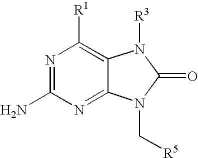 | |

| No. | Ex | R¹ | R³ | R⁵ |
|---|---|---|---|---|
| 1 | 10 | Cl | H | 3,5-Dimethyl-4-methoxypyridin-2-yl |
| 2 | 13 | Cl | Me | 3,5-Dimethyl-4-methoxypyridin-2-yl |
| 3 | 14 | Cl | Et | 3,5-Dimethyl-4-methoxypyridin-2-yl |
| 4 | 15 | Cl | i-Pr | 3,5-Dimethyl-4-methoxypyridin-2-yl |
| 5 | 16 | Cl | n-Bu | 3,5-Dimethyl-4-methoxypyridin-2-yl |
| 6 | 17 | Cl | n-Heptyl | 3,5-Dimethyl-4-methoxypyridin-2-yl |
| 7 | 18 | Cl | Bn | 3,5-Dimethyl-4-methoxypyridin-2-yl |
| 8 | 19 | Cl | n-Pentyl | 3,5-Dimethyl-4-methoxypyridin-2-yl |
| 9 | 20 | Cl | 4-methyl-pent-3-enyl | 3,5-Dimethyl-4-methoxypyridin-2-yl |
| 10 | 21 | Cl | 2-dimethyl amino-ethyl | 3,5-Dimethyl-4-methoxypyridin-2-yl |
| 11 | 22 | Cl | 2-acetoxy-ethyl | 3,5-Dimethyl-4-methoxypyridin-2-yl |
| 12 | 23 | Cl | 3-pyrrol-1-yl-propyl | 3,5-Dimethyl-4-methoxypyridin-2-yl |
| 13 | 24 | Cl | 3,5-dimethoxy-benzyl | 3,5-Dimethyl-4-methoxypyridin-2-yl |
| 14 | 25 | Cl | 2-morpholin-4-yl-ethyl | 3,5-Dimethyl-4-methoxypyridin-2-yl |
| 15 | 26 | Cl | Pent-4-ynyl | 3,5-Dimethyl-4-methoxypyridin-2-yl |
| 16 | 27 | Cl | 3,4-dimethoxy-pyridin-2-ylmethyl | 3,5-Dimethyl-4-methoxypyridin-2-yl |
| 17 | 28 | Cl | 2-methyl-thiazol-4-ylmethyl | 3,5-Dimethyl-4-methoxypyridin-2-yl |
| 18 | 29 | Cl | 4-fluoro-2-trifluoromethyl-benzyl | 3,5-Dimethyl-4-methoxypyridin-2-yl |
| 19 | 30 | Cl | Ethoxycarbonyl-methyl | 3,5-Dimethyl-4-methoxypyridin-2-yl |
| 20 | 31 | Cl | 3-Acetoxy-propyl | 3,5-Dimethyl-4-methoxypyridin-2-yl |
| 21 | 32 | Cl | 3-hydroxy-propyl | 3,5-Dimethyl-4-methoxypyridin-2-yl |
| 22 | 33 | Cl | 2-Phenyl-ethyl | 3,5-Dimethyl-4-methoxypyridin-2-yl |
| 23 | 34 | Cl | Prop-2-ynyl | 3,5-Dimethyl-4-methoxypyridin-2-yl |
| 24 | 35 | Cl | Pent-4-enyl | 3,5-Dimethyl-4-methoxypyridin-2-yl |
| 25 | 36 | Cl | n-Hexyl | 3,5-Dimethyl-4-methoxypyridin-2-yl |
| 26 | 37 | Cl | 4-Methyl-pentyl | 3,5-Dimethyl-4-methoxypyridin-2-yl |
| 27 | 38 | Cl | 2,3,6-Trifluoro-benzyl | 3,5-Dimethyl-4-methoxypyridin-2-yl |
| 28 | 39 | Cl | Pent-2-ynyl | 3,5-Dimethyl-4-methoxypyridin-2-yl |
| 29 | 40 | Cl | 3-Bromo-propyl | 3,5-Dimethyl-4-methoxypyridin-2-yl |
| 30 | 41 | Cl | 3-Cyano-propyl | 3,5-Dimethyl-4-methoxypyridin-2-yl |
| 31 | 42 | Cl | 3-Methyl-but-2-enyl | 3,5-Dimethyl-4-methoxypyridin-2-yl |
| 32 | 43 | Cl | 2-Diethylamino-ethyl | 3,5-Dimethyl-4-methoxypyridin-2-yl |
| 33 | 44 | Cl | 2-Methylsulfanyl-ethyl | 3,5-Dimethyl-4-methoxypyridin-2-yl |
| 34 | 45 | Cl | n-Propyl | 3,5-Dimethyl-4-methoxypyridin-2-yl |
| 35 | 46 | Cl | 2-Methoxy-ethyl | 3,5-Dimethyl-4-methoxypyridin-2-yl |
| 36 | 47 | Cl | But-3-enyl | 3,5-Dimethyl-4-methoxypyridin-2-yl |
| 37 | 48 | Cl | 3,4-Dichloro-benzyl | 3,5-Dimethyl-4-methoxypyridin-2-yl |
| 38 | 49 | Cl | 3-Diethylamino-propyl | 3,5-Dimethyl-4-methoxypyridin-2-yl |
| 39 | 50 | Cl | Tetrahydro-pyran-2-ylmethyl | 3,5-Dimethyl-4-methoxypyridin-2-yl |
| 40 | 51 | Cl | 2-(1-methyl-pyrrolidin-2-yl)-ethyl | 3,5-Dimethyl-4-methoxypyridin-2-yl |
| 41 | 52 | Cl | 2-Ethoxy-ethyl | 3,5-Dimethyl-4-methoxypyridin-2-yl |
| 42 | 53 | Cl | i-Pentyl | 3,5-Dimethyl-4-methoxypyridin-2-yl |
| 43 | 54 | Cl | Hex-5-enyl | 3,5-Dimethyl-4-methoxypyridin-2-yl |
| 44 | 55 | Cl | Hex-5-ynyl | 3,5-Dimethyl-4-methoxypyridin-2-yl |
| 45 | 56 | Cl | 1H-Benzoimidazol-2-ylmethyl | 3,5-Dimethyl-4-methoxypyridin-2-yl |
| 46 | 57 | Cl | 2-(Piperidin-1-yl)-ethyl | 3,5-Dimethyl-4-methoxypyridin-2-yl |
| 47 | 58 | Cl | 2-(Pyrrolidin-1-yl)-ethyl | 3,5-Dimethyl-4-methoxypyridin-2-yl |
| 48 | 59 | Cl | 2-Diisopropyl amino-ethyl | 3,5-Dimethyl-4-methoxypyridin-2-yl |
| 49 | 60 | Cl | 2-Fluoro-benzyl | 3,5-Dimethyl-4-methoxypyridin-2-yl |
| 50 | 61 | Cl | 3-Nitro-benzyl | 3,5-Dimethyl-4-methoxypyridin-2-yl |
| 51 | 62 | Cl | 2-Bromo-ethyl | 3,5-Dimethyl-4-methoxypyridin-2-yl |
| 52 | 63 | Cl | 3,5-Dimethyl-4-methoxypyridin-2-ylmethyl | 3,5-Dimethyl-4-methoxypyridin-2-yl |
| 53 | 64 | Cl | Cyclohexyl-methyl | 3,5-Dimethyl-4-methoxypyridin-2-yl |
| 54 | 65 | Cl | 3-Fluoro-benzyl | 3,5 -Dimethyl-4-methoxypyridin-2-yl |
| 55 | 66 | Cl | 4-Fluoro-benzyl | 3,5-Dimethyl-4-methoxypyridin-2-yl |
| 56 | 67 | Cl | 2-Methyl-benzyl | 3,5-Dimethyl-4-methoxypyridin-2-yl |
| 57 | 68 | Cl | 4-Methyl-benzyl | 3,5-Dimethyl-4-methoxypyridin-2-yl |
| 58 | 69 | Cl | 4-Acetoxy-butyl | 3,5-Dimethyl-4-methoxypyridin-2-yl |
| 59 | 70 | Cl | Cyano-methyl | 3,5-Dimethyl-4-methoxypyridin-2-yl |
| 60 | 71 | Cl | 3,4,4-Trifluoro-but-3-enyl | 3,5-Dimethyl-4-methoxypyridin-2-yl |
| 61 | 72 | Cl | 3-Methoxy-benzyl | 3,5-Dimethyl-4-methoxypyridin-2-yl |
| 62 | 73 | Cl | 4-Methoxy-benzyl | 3,5-Dimethyl-4-methoxypyridin-2-yl |
| 63 | 74 | Cl | 2-Oxo-oxazolidin-5-ylmethyl | 3,5-Dimethyl-4-methoxypyridin-2-yl |
| 64 | 75 | Cl | 3-(Diethoxy-phosphoryl)-propyl | 3,5-Dimethyl-4-methoxypyridin-2-yl |
| 65 | 76 | Cl | 3-Methyl-benzyl | 3,5-Dimethyl-4-methoxypyridin-2-yl |
| 66 | 77 | Cl | 3-Isopropylamino-propyl | 3,5-Dimethyl-4-methoxypyridin-2-yl |
| 67 | 78 | Cl | 3-Isobutylamino-propyl | 3,5-Dimethyl-4-methoxypyridin-2-yl |
| 68 | 79 | Cl | 3,4-Difluoro-benzyl | 3,5-Dimethyl-4-methoxypyridin-2-yl |
| 69 | 80 | Cl | 4-Methoxy carbonyl-benzyl | 3,5-Dimethyl-4-methoxypyridin-2-yl |
| 70 | 81 | Cl | Pent-2-enyl | 3,5-Dimethyl-4-methoxypyridin-2-yl |
| 71 | 82 | Cl | Pyridin-2-ylmethyl | 3,5-Dimethyl-4-methoxypyridin-2-yl |
| 72 | 83 | Cl | Pyridin-3-ylmethyl | 3,5-Dimethyl-4-methoxypyridin-2-yl |
| 73 | 84 | Cl | Pyridin-4-ylmethyl | 3,5-Dimethyl-4-methoxypyridin-2-yl |
| 74 | 85 | Cl | Methoxycarbonyl-propyl | 3,5-Dimethyl-4-methoxypyridin-2-yl |
| 75 | 86 | Cl | 2-(2,4-Dioxo-1,4-dihydro-2H-quinazolin-3-yl)-ethyl | 3,5-Dimethyl-4-methoxypyridin-2-yl |
| 76 | 87 | Cl | 2-Isopropylamino-ethyl | 3,5-Dimethyl-4-methoxypyridin-2-yl |
| 77 | 88 | Cl | 2-Amino-ethyl | 3,5-Dimethyl-4-methoxypyridin-2-yl |
| 78 | 89 | Cl | 2-Pyrrol-1-yl-ethyl | 3,5-Dimethyl-4-methoxypyridin-2-yl |
| 79 | 90 | Cl | Acetyl | 3,5-Dimethyl-4-methoxypyridin-2-yl |
| 80 | 91 | Cl | 2-[1,2,4]Triazol-1-yl-ethyl | 3,5-Dimethyl-4-methoxypyridin-2-yl |
| 81 | 92 | Cl | 2-(4-Methyl-4H-[1,2,4]triazol-3-ylsulfanyl)-ethyl | 3,5-Dimethyl-4-methoxypyridin-2-yl |
| 82 | 93 | Cl | 2-(Imidazol-1-yl)-ethyl | 3,5-Dimethyl-4-methoxypyridin-2-yl |
| 83 | 94 | Cl | 2-(4-Methyl-piperazin-1-yl)-ethyl | 3,5-Dimethyl-4-methoxypyridin-2-yl |
| 84 | 95 | Cl | Pentanoyl | 3,5-Dimethyl-4-methoxypyridin-2-yl |
| 85 | 100 | Cl | 2-(5-Amino-tetrazol-1-yl)-ethyl | 3,5-Dimethyl-4-methoxypyridin-2-yl |
| 86 | 101 | Cl | 2-Oxo-benzothiazol-3-ylmethyl | 3,5-Dimethyl-4-methoxypyridin-2-yl |
| 87 | 96 | Cl | 2-Pyrrol-1-yl-ethyl | 3,5-Dimethyl-4-iodopyridin-2-yl |
| 88 | 6 | Cl | H | 3,5-Dimethyl-4-iodopyridin-2-yl |
| 89 | 97 | Cl | 4-Methyl-pent-3-enyl | 3,5-Dimethyl-4-iodopyridin-2-yl |
| 90 | 98 | Cl | 2-Diisopropyl amino-ethyl | 3,5-Dimethyl-4-iodopyridin-2-yl |
| 91 | 99 | Cl | Pyridin-2-ylmethyl | 3,5-Dimethyl-4-iodopyridin-2-yl |
| 92 | 102 | Cl | 2-(4-methyl-piperazin-1-yl)-ethyl | 3,5-dimethyl-4-iodopyridin-2-yl |
| 93 | 103 | Cl | 2-bromoethyl | 3,5-dimethyl-4-iodopyridin-2-yl |
| 94 | 104 | Cl | 2-(4-(ethoxycarbonyl)piperazin-1-yl)-ethyl | 3,5-dimethyl-4-iodopyridin-2-yl |
| 95 | 105 | Cl | Acetyl | 3,5-dimethyl-4-iodopyridin-2-yl |
| 96 | 106 | Cl | 2-diisobutylamino-ethyl | 3,5-dimethyl-4-iodopyridin-2-yl |
| 97 | 107 | Cl | 2-(dipropylamino)ethyl | 3,5-dimethyl-4-iodopyridin-2-yl |
| 98 | 108 | Cl | 2-(isopropyl(methyl)amino)ethyl | 3,5-dimethyl-4-iodopyridin-2-yl |
| 99 | 109 | Cl | 2-(diisopropylamino)ethyl | N-oxide-3,5-dimethyl-4-iodopyridin-2-yl |
| 100 | 4 | Cl | H | 3,5-dimethyl-4-bromopyridin-2-yl |
| 101 | 110 | Cl | 2-(diisopropylamino)ethyl | 3,5-dimethyl-4-bromopyridin-2-yl |
| 102 | 111 | Cl | 4-methylpent-3-enyl | 3,5-dimethyl-4-bromopyridin-2-yl |
| 103 | 112 | Cl | 2-(1H-pyrrol-1-yl)ethyl | 3,5-dimethyl-4-bromopyridin-2-yl |
| 104 | 8 | Cl | H | 2-bromo-3,4,5-trimethoxyphenyl |
| 105 | 113 | Cl | 2-(diisopropylamino)ethyl | 2-bromo-3,4,5-trimethoxyphenyl |
| 106 | 114 | Cl | 4-methylpent-3-enyl | 2-bromo-3,4,5-trimethoxyphenyl |
| 107 | 115 | Cl | 2-(1H-pyrrol-1-yl)ethyl | 2-bromo-3,4,5-trimethoxyphenyl |
| 108 | 10 | Cl | H | 4,6-dimethyl-5-methoxypyridin-3-yl |
| 109 | 116 | Cl | 4-methylpent-3-enyl | 4,6-dimethyl-5-methoxypyridin-3-yl |
| 110 | 117 | Cl | 2-(1H-pyrrol-1-yl)ethyl | 4,6-dimethyl-5-methoxypyridin-3-yl |
| 111 | 118 | Cl | 2-(diisopropylamino)ethyl | 4,6-dimethyl-5-methoxypyridin-3-yl |
| 112 | | Cl | H | 2-chloro-3,4,5-trimethoxyphenyl |
| 113 | | Cl | H | 2-chloro-3,5-dimethoxy-4-methylphenyl |
| 114 | | Cl | H | 2-bromo-3,5-dimethoxy-4-methylphenyl |
| 115 | | Cl | H | 2-chloro-4-methoxy-3,5-dimethylphenyl |
| 116 | | Cl | H | 2-bromo-4-methoxy-3,5-dimethylphenyl |
| 117 | | Cl | H | 3,4,5-trimethoxypyridin-2-yl |
| 118 | | Cl | H | N-oxide-3,4,5-trimethoxypyridin-2-yl |
| 119 | | Cl | H | 3,4,5-trimethylpyridin-2-yl |
| 120 | | Cl | H | N-oxide-3,4,5-trimethylpyridin-2-yl |
| 121 | | Cl | H | 3-chloro-4,5,6-trimethoxypyridin-2-yl |
| 122 | | Cl | H | N-oxide-3-chloro-4,5,6-trimethoxypyridin-2-yl |
| 123 | | Cl | H | 3-bromo-4,5,6-trimethoxypyridin-2-yl |
| 124 | | Cl | H | N-oxide-3-bromo-4,5,6-trimethoxypyridin-2-yl |
| 125 | | Cl | H | N-oxide-3-bromo-4,5,6-trimethoxypyridin-2-yl |
| 126 | | Br | H | 4-methoxy-3,5-dimethylpyridin-2-yl |
| 127 | | Br | H | N-oxide-4-methoxy-3,5-dimethylpyridin-2-yl |
| 128 | | Br | H | 5-methoxy-4,6-dimethylpyridin-3-yl |
| 129 | | Br | H | N-oxide-5-methoxy-4,6-dimethylpyridin-3-yl |
| 130 | | Br | H | 3,4,5-trimethoxypyridin-2-yl |
| 131 | | Br | H | N-oxide-3,4,5-trimethoxypyridin-2-yl |
| 132 | | Br | H | 3,4,5-trimethylpyridin-2-yl |
| 133 | | Br | H | N-oxide-3,4,5-trimethylpyridin-2-yl |
| 134 | | Br | H | 3-chloro-4,5,6-trimethoxypyridin-2-yl |
| 135 | | Br | H | N-oxide-3-chloro-4,5,6-trimethoxypyridin-2-yl |
| 136 | | Br | H | 3-bromo-4,5,6-trimethoxypyridin-2-yl |
| 137 | | Br | H | N-oxide-3-bromo-4,5,6-trimethoxypyridin-2-yl |
| 138 | | Cl | H | 6-chloro-4-methoxy-3,5-dimethylpyridin-2-yl |
| 139 | | Cl | H | 6-bromo-4-methoxy-3,5-dimethylpyridin-2-yl |
| 140 | | Cl | H | 6-methoxy-4-methoxy-3,5-dimethylpyridin-2-yl |
| 141 | | Cl | H | N-oxide-6-chloro-4-methoxy-3,5-dimethylpyridin-2-yl |
| 142 | | Cl | H | N-oxide-6-bromo-4-methoxy-3,5-dimethylpyridin-2-yl |
| 143 | | Cl | H | N-oxide-6-methoxy-4-methoxy-3,5-dimethylpyridin-2-yl |
| 144 | | Cl | 4-methylpent-3-enyl | 2-chloro-3,4,5-trimethoxyphenyl |
| 145 | | Cl | 4-methylpent-3-enyl | 2-chloro-3,5-dimethoxy-4-methylphenyl |
| 146 | | Cl | 4-methylpent-3-enyl | 2-bromo-3,5-dimethoxy-4-methylphenyl |
| 147 | | Cl | 4-methylpent-3-enyl | 2-chloro-4-methoxy-3,5-dimethylphenyl |
| 148 | | Cl | 4-methylpent-3-enyl | 2-bromo-4-methoxy-3,5-dimethylphenyl |
| 149 | | Cl | 4-methylpent-3-enyl | 3,4,5-trimethoxypyridin-2-yl |
| 150 | | Cl | 4-methylpent-3-enyl | N-oxide-3,4,5-trimethoxypyridin-2-yl |
| 151 | | Cl | 4-methylpent-3-enyl | 3,4,5-trimethylpyridin-2-yl |
| 152 | | Cl | 4-methylpent-3-enyl | N-oxide-3,4,5-trimethylpyridin-2-yl |
| 153 | | Cl | 4-methylpent-3-enyl | 3-chloro-4,5,6-trimethoxypyridin-2-yl |
| 154 | | Cl | 4-methylpent-3-enyl | N-oxide-3-chloro-4,5,6-trimethoxypyridin-2-yl |
| 155 | | Cl | 4-methylpent-3-enyl | 3-bromo-4,5,6-trimethoxypyridin-2-yl |
| 156 | | Cl | 4-methylpent-3-enyl | N-oxide-3-bromo-4,5,6-trimethoxypyridin-2-yl |
| 157 | | Cl | 4-methylpent-3-enyl | N-oxide-3-bromo-4,5,6-trimethoxypyridin-2-yl |
| 158 | | Br | 4-methylpent-3-enyl | 4-methoxy-3,5-dimethylpyridin-2-yl |
| 159 | | Br | 4-methylpent-3-enyl | N-oxide-4-methoxy-3,5-dimethylpyridin-2-yl |
| 160 | | Br | 4-methylpent-3-enyl | 5-methoxy-4,6-dimethylpyridin-3-yl |
| 161 | | Br | 4-methylpent-3-enyl | N-oxide-5-methoxy-4,6-dimethylpyridin-3-yl |
| 162 | | Br | 4-methylpent-3-enyl | 3,4,5-trimethoxypyridin-2-yl |
| 163 | | Br | 4-methylpent-3-enyl | N-oxide-3,4,5-trimethoxypyridin-2-yl |
| 164 | | Br | 4-methylpent-3-enyl | 3,4,5-trimethylpyridin-2-yl |
| 165 | | Br | 4-methylpent-3-enyl | N-oxide-3,4,5-trimethylpyridin-2-yl |
| 166 | | Br | 4-methylpent-3-enyl | 3-chloro-4,5,6-trimethoxypyridin-2-yl |
| 167 | | Br | 4-methylpent-3-enyl | N-oxide-3-chloro-4,5,6-trimethoxypyridin-2-yl |
| 168 | | Br | 4-methylpent-3-enyl | 3-bromo-4,5,6-trimethoxypyridin-2-yl |
| 169 | | Br | 4-methylpent-3-enyl | N-oxide-3-bromo-4,5,6-trimethoxypyridin-2-yl |
| 170 | | Cl | 4-methylpent-3-enyl | 6-chloro-4-methoxy-3,5-dimethylpyridin-2-yl |
| 171 | | Cl | 4-methylpent-3-enyl | 6-bromo-4-methoxy-3,5-dimethylpyridin-2-yl |
| 172 | | Cl | 4-methylpent-3-enyl | 6-methoxy-4-methoxy-3,5-dimethylpyridin-2-yl |
| 173 | | Cl | 4-methylpent-3-enyl | N-oxide-6-chloro-4-methoxy-3,5-dimethylpyridin-2-yl |
| 174 | | Cl | 4-methylpent-3-enyl | N-oxide-6-bromo-4-methoxy-3,5-dimethylpyridin-2-yl |
| 175 | | Cl | 4-methylpent-3-enyl | N-oxide-6-methoxy-4-methoxy-3,5-dimethylpyridin-2-yl |
| 176 | | Cl | 2-(1H-pyrrol-1-yl)ethyl | 2-chloro-3,4,5-trimethoxyphenyl |
| 177 | | Cl | 2-(1H-pyrrol-1-yl)ethyl | 2-chloro-3,5-dimethoxy-4-methylphenyl |
| 178 | | Cl | 2-(1H-pyrrol-1-yl)ethyl | 2-bromo-3,5-dimethoxy-4-methylphenyl |
| 179 | | Cl | 2-(1H-pyrrol-1-yl)ethyl | 2-chloro-4-methoxy-3,5-dimethylphenyl |
| 180 | | Cl | 2-(1H-pyrrol-1-yl)ethyl | 2-bromo-4-methoxy-3,5-dimethylphenyl |
| 181 | | Cl | 2-(1H-pyrrol-1-yl)ethyl | 3,4,5-trimethoxypyridin-2-yl |
| 182 | | Cl | 2-(1H-pyrrol-1-yl)ethyl | N-oxide-3,4,5-trimethoxypyridin-2-yl |
| 183 | | Cl | 2-(1H-pyrrol-1-yl)ethyl | 3,4,5-trimethylpyridin-2-yl |
| 184 | | Cl | 2-(1H-pyrrol-1-yl)ethyl | N-oxide-3,4,5-trimethylpyridin-2-yl |
| 185 | | Cl | 2-(1H-pyrrol-1-yl)ethyl | 3-chloro-4,5,6-trimethoxypyridin-2-yl |
| 186 | | Cl | 2-(1H-pyrrol-1-yl)ethyl | N-oxide-3-chloro-4,5,6-trimethoxypyridin-2-yl |
| 187 | | Cl | 2-(1H-pyrrol-1-yl)ethyl | 3-bromo-4,5,6-trimethoxypyridin-2-yl |
| 188 | | Cl | 2-(1H-pyrrol-1-yl)ethyl | N-oxide-3-bromo-4,5,6-trimethoxypyridin-2-yl |
| 189 | | Cl | 2-(1H-pyrrol-1-yl)ethyl | N-oxide-3-bromo-4,5,6-trimethoxypyridin-2-yl |
| 190 | | Br | 2-(1H-pyrrol-1-yl)ethyl | 4-methoxy-3,5-dimethylpyridin-2-yl |
| 191 | | Br | 2-(1H-pyrrol-1-yl)ethyl | N-oxide-4-methoxy-3,5-dimethylpyridin-2-yl |
| 192 | | Br | 2-(1H-pyrrol-1-yl)ethyl | 5-methoxy-4,6-dimethylpyridin-3-yl |
| 193 | | Br | 2-(1H-pyrrol-1-yl)ethyl | N-oxide-5-methoxy-4,6-dimethylpyridin-3-yl |
| 194 | | Br | 2-(1H-pyrrol-1-yl)ethyl | 3,4,5-trimethoxypyridin-2-yl |
| 195 | | Br | 2-(1H-pyrrol-1-yl)ethyl H | N-oxide-3,4,5-trimethoxypyridin-2-yl |
| 196 | | Br | 2-(1H-pyrrol-1-yl)ethyl H | 3,4,5-trimethylpyridin-2-yl |
| 197 | | Br | 2-(1H-pyrrol-1-yl)ethyl | N-oxide-3,4,5-trimethylpyridin-2-yl |
| 198 | | Br | 2-(1H-pyrrol-1-yl)ethyl | 3-chloro-4,5,6-trimethoxypyridin-2-yl |
| 199 | | Br | 2-(1H-pyrrol-1-yl)ethyl | N-oxide-3-chloro-4,5,6-trimethoxypyridin-2-yl |
| 200 | | Br | 2-(1H-pyrrol-1-yl)ethyl | 3-bromo-4,5,6-trimethoxypyridin-2-yl |
| 201 | | Br | 2-(1H-pyrrol-1-yl)ethyl | N-oxide-3-bromo-4,5,6-trimethoxypyridin-2-yl |
| 202 | | Cl | 2-(1H-pyrrol-1-yl)ethyl | 6-chloro-4-methoxy-3,5-dimethylpyridin-2-yl |
| 203 | | Cl | 2-(1H-pyrrol-1-yl)ethyl | 6-bromo-4-methoxy-3,5-dimethylpyridin-2-yl |
| 204 | | Cl | 2-(1H-pyrrol-1-yl)ethyl | 6-methoxy-4-methoxy-3,5-dimethylpyridin-2-yl |
| 205 | | Cl | 2-(1H-pyrrol-1-yl)ethyl | N-oxide-6-chloro-4-methoxy-3,5-dimethylpyridin-2-yl |
| 206 | | Cl | 2-(1H-pyrrol-1-yl)ethyl | N-oxide-6-bromo-4-methoxy-3,5-dimethylpyridin-2-yl |
| 207 | | Cl | 2-(1H-pyrrol-1-yl)ethyl | N-oxide-6-methoxy-4-methoxy-3,5-dimethylpyridin-2-yl |
| 208 | | Cl | 2-(diisopropylamino)ethyl | 2-chloro-3,4,5-trimethoxyphenyl |
| 209 | | Cl | 2-(diisopropylamino)ethyl | 2-chloro-3,5-dimethoxy-4-methylphenyl |
| 210 | | Cl | 2-(diisopropylamino)ethyl | 2-bromo-3,5-dimethoxy-4-methylphenyl |
| 211 | | Cl | 2-(diisopropylamino)ethyl | 2-chloro-4-methoxy-3,5-dimethylphenyl |
| 212 | | Cl | 2-(diisopropylamino)ethyl | 2-bromo-4-methoxy-3,5-dimethylphenyl |
| 213 | | Cl | 2-(diisopropylamino)ethyl | 3,4,5-trimethoxypyridin-2-yl |
| 214 | | Cl | 2-(diisopropylamino)ethyl | N-oxide-3,4,5-trimethoxypyridin-2-yl |
| 215 | | Cl | 2-(diisopropylamino)ethyl | 3,4,5-trimethylpyridin-2-yl |
| 216 | | Cl | 2-(diisopropylamino)ethyl | N-oxide-3,4,5-trimethylpyridin-2-yl |
| 217 | | Cl | 2-(diisopropylamino)ethyl | 3-chloro-4,5,6-trimethoxypyridin-2-yl |
| 218 | | Cl | 2-(diisopropylamino)ethyl | N-oxide-3-chloro-4,5,6-trimethoxypyridin-2-yl |
| 219 | | Cl | 2-(diisopropylamino)ethyl | 3-bromo-4,5,6-trimethoxypyridin-2-yl |
| 220 | | Cl | 2-(diisopropylamino)ethyl | N-oxide-3-bromo-4,5,6-trimethoxypyridin-2-yl |
| 221 | | Cl | 2-(diisopropylamino)ethyl | N-oxide-3-bromo-4,5,6-trimethoxypyridin-2-yl |
| 222 | | Br | 2-(diisopropylamino)ethyl | 4-methoxy-3,5-dimethylpyridin-2-yl |
| 223 | | Br | 2-(diisopropylamino)ethyl | N-oxide-4-methoxy-3,5-dimethylpyridin-2-yl |
| 224 | | Br | 2-(diisopropylamino)ethyl | 5-methoxy-4,6-dimethylpyridin-3-yl |
| 225 | | Br | 2-(diisopropylamino)ethyl | N-oxide-5-methoxy-4,6-dimethylpyridin-3-yl |
| 226 | | Br | 2-(diisopropylamino)ethyl | 3,4,5-trimethoxypyridin-2-yl |
| 227 | | Br | 2-(diisopropylamino)ethyl | N-oxide-3,4,5-trimethoxypyridin-2-yl |
| 228 | | Br | 2-(diisopropylamino)ethyl | 3,4,5-trimethylpyridin-2-yl |
| 229 | | Br | 2-(diisopropylamino)ethyl | N-oxide-3,4,5-trimethylpyridin-2-yl |
| 230 | | Br | 2-(diisopropylamino)ethyl | 3-chloro-4,5,6-trimethoxypyridin-2-yl |
| 231 | | Br | 2-(diisopropylamino)ethyl | N-oxide-3-chloro-4,5,6-trimethoxypyridin-2-yl |
| 232 | | Br | 2-(diisopropylamino)ethyl | 3-bromo-4,5,6-trimethoxypyridin-2-yl |
| 233 | | Br | 2-(diisopropylamino)ethyl | N-oxide-3-bromo-4,5,6-trimethoxypyridin-2-yl |
| 234 | | Cl | 2-(diisopropylamino)ethyl | 6-chloro-4-methoxy-3,5-dimethylpyridin-2-yl |
| 235 | | Cl | 2-(diisopropylamino)ethyl | 6-bromo-4-methoxy-3,5-dimethylpyridin-2-yl |
| 236 | | Cl | 2-(diisopropylamino)ethyl | 6-methoxy-4-methoxy-3,5-dimethylpyridin-2-yl |
| 237 | | Cl | 2-(diisopropylamino)ethyl | N-oxide-6-chloro-4-methoxy-3,5-dimethylpyridin-2-yl |
| 238 | | Cl | 2-(diisopropylamino)ethyl | N-oxide-6-bromo-4-methoxy-3,5-dimethylpyridin-2-yl |
| 239 | | Cl | 2-(diisopropylamino)ethyl | N-oxide-6-methoxy-4-methoxy-3,5-dimethylpyridin-2-yl |

III. Synthesis of the Compounds of the Invention

The compounds of Formula I of the present invention may be synthesized by various methods known in the art. The general strategy is outlined in Scheme 1 and consists of three parts:

Starting from the pyrimidine 1: (1) Appending the R⁵—R⁴NH group (2) Constructing the bicyclic system by forming the 5-membered ring and (3) further elaborating the ring systems.

Starting from the imidazolopyrimidine 4: (4) Appending the R⁵—R⁴ group (5) Oxidizing the 5 membered ring and (3) further elaborating the ring systems

Importantly, one skilled in the art will recognize that the sequence of events is not necessarily (1) -(2)-(3), and (4)-(5)-(3) and that these events may be interchanged, provided there be no incompatibility between the reagents and the functional groups specific to the case in point.

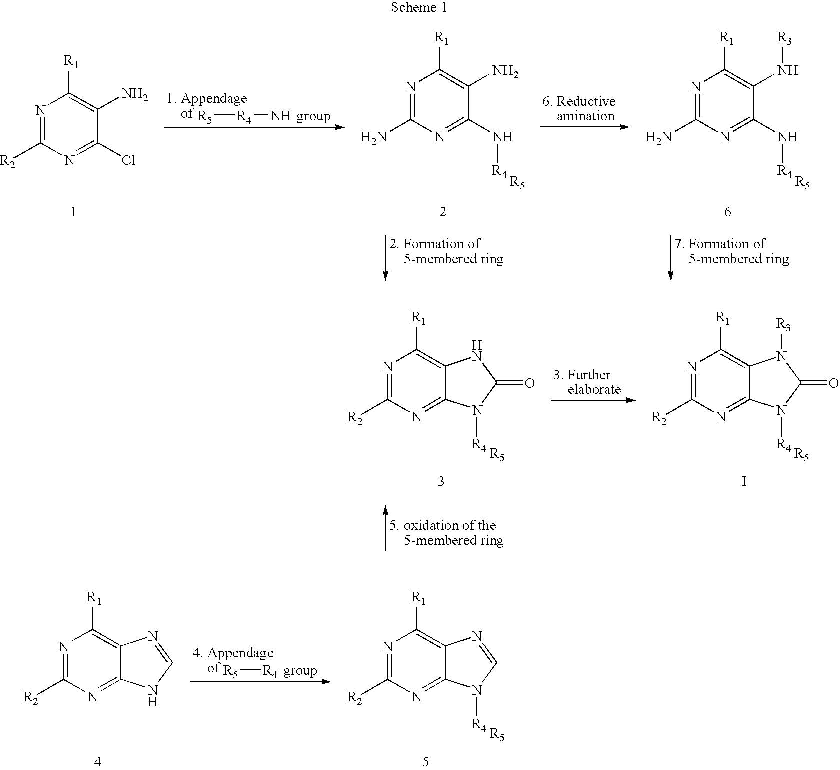

It should be noted that many of the compounds of the present invention can exist in various tautomeric forms, as illustrated by intermediate 3 and compound I in FIG. 1. Any form of a particular compound is used indiscriminately in this patent, and it should be assumed that all forms are intended to be covered, regardless of the particular form used in an illustration.

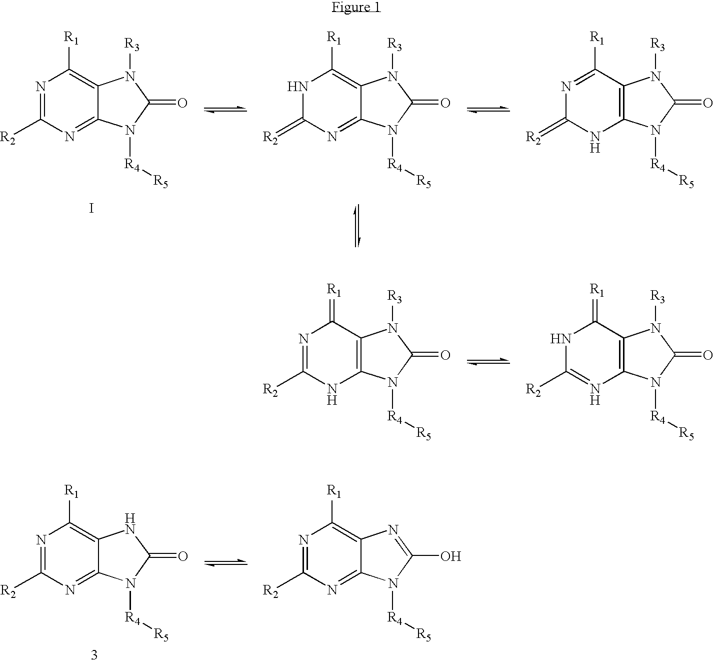

1. Incorporation of the R⁵—R⁴— Group
1.1. Synthesis of Pyridyl Methyl Type Nucleophile

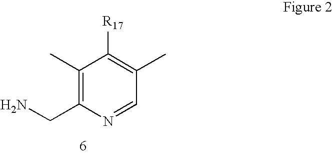

The compound of formula 6 wherein R17 is OMe (FIG. 2) can be made by nucleophilic displacement of L1-R4-R5 with ammonia or sodium azide followed with hydrogenolysis or phtalimide followed by hydrazine treatment (Kasibhatla, patent publication number US2005/01113339 A1).
1.2. Functional Group Interconversion of R17

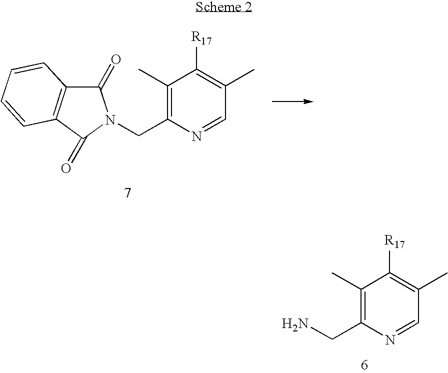

The compound of formula 7 wherein R17 is OMe can be hydrolyzed to give a compound of formula 7 wherein R17 is OH.

The compound of formula 7 wherein R17 is OH can be converted into a compound of formula 7 wherein R17 is Cl, Br, I.

The compound of formula 7 wherein R17 is Cl, Br, I can be converted to the compound of formula 6 wherein R17 is Cl, Br, I by hydrazine treatment.
1.3 Incorporation of R5-R4-NH Group by Nucleophilic Substitution on 4-Chloro-Pyrimidine.

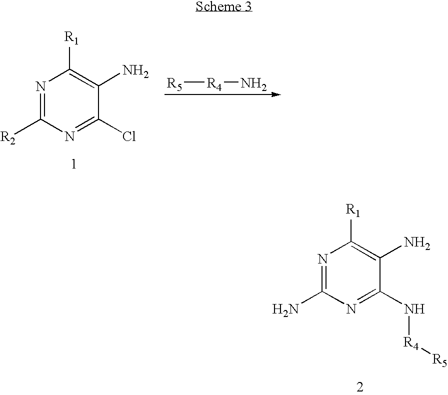

The compound of formula 2 wherein R1 is C1 and R2 is NH2 can be made by treating the compound of formula I with R5-R4-NH2 upon heating in protic solvents in presence of base such as triethylamine or diisopropylethylamine (M. L. Sznaidman J. Heterocycl. Chem. 1996, 33, 1610; Kasibhatla, publication number US2005/0113339 A1).
1.4 Incorporation of R5-R4 Group by Alkylation of Purines.

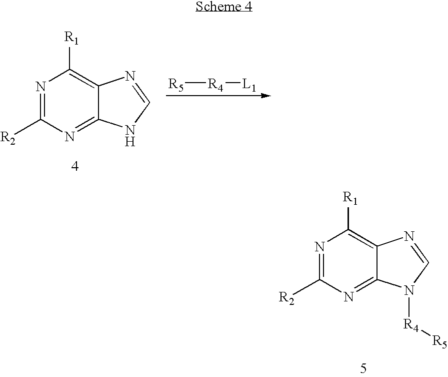

Compounds of Formula 5 can be made by alkylation of compound of formula 4 in the presence of a base such as K2CO3, NaH, Cs2CO3, DBU etc. with/without the presence of a catalyst such as NaI, KI, (Bu)4NI etc., and in a polar solvent such as DMF, THF, DMSO etc. using electrophiles such as L1-R4-R5 where L1 is a leaving group. Leaving groups include but are not limited to, e.g., halogen, triflate, tosylate, mesylate, triphenylphosphonium (generated under Mitsunobu conditions, e.g. PPh3/DEAD) etc. (Kasibhatla, PCT publication number WO 03/037860).

2. Assembly of the 7,9-dihydro-purin-8-one

2.1. Assembly of the 7,9-dihydro-purin-8-one

The compounds of Formula 3 can be prepared from pyrimidines or from purines as outlined in Scheme 5. For instance:

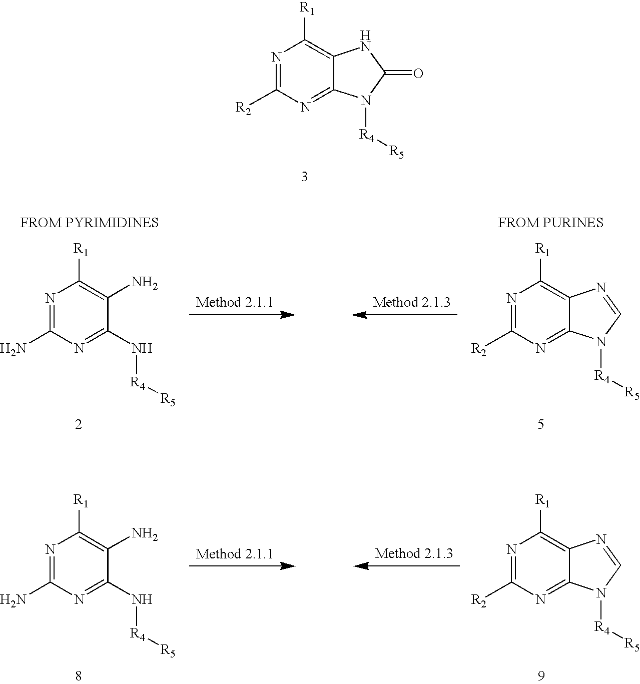

Method 2.1.1

The compound of formula 3 can be made by intramolecular cyclization of compound of formula 2 using phosgene, carbonyl diimidazole, diethyl carbonate or related reagents such as diphosgene or triphosgene in presence of a base such as diisopropylethylamine. (A. B. Reitz J. Med. Chem. 1994, 37, 3561).

Method 2.1.2:

The compound of formula 3 can be made by intramolecular cyclization of compound of formula 8 in presence of a base such as K2CO3, Cs2CO3, MeONa or (iPr)2NEt. (K. S. Atwal J. Med. Chem. 1995, 38, 3236).

Method 2.1.3:

The compound of formula 3 can be made by chlorination or bromination of compound of formula 9 using chlorine or bromine (Z. Janeba Collect. Czech. Chem. Commun. 2000, 1126) followed either by treatment with an aqueous base such as NaOH 1N or KOH IN upon heating (T. Fujii Chem. Pharm. Bull. 1990, 38, 2146) or by heating in presence of AcOH (T. Maruyama Nucleosides Nucleotides 2000, 1193) or by irradiation in ethanol. (G. Crank Aust. J. Chem. 1982, 35, 775).

Method 2.1.4:

Compounds of Formula 3 can be made by alkylation of compound of formula 9 in the presence of a base such as K2CO3, NaH, Cs2CO3, DBU etc. with/without the presence of a catalyst such as NaI, KI, (Bu)4NI etc., and in a polar solvent such as DMF, THF, DMSO etc. using electrophiles such as L1-R4-R5 where L1 is a leaving group. Leaving groups include but are not limited to, e.g., halogen, triflate, tosylate, mesylate, triphenylphosphonium (generated under Mitsunobu conditions, e.g. PPh3/DEAD) etc. (Kasibhatla, PCT publication number WO 03/037860).

3. Further Elaboration of the Ring Systems
3.1. Incorporation of R³ Fragment

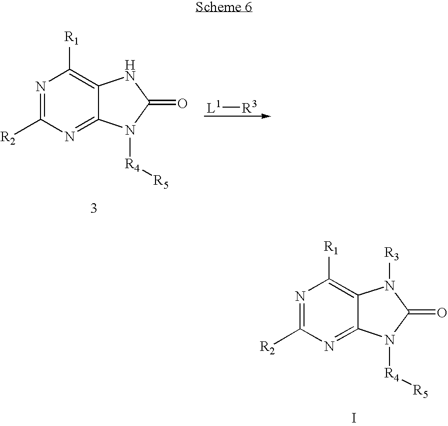

Compounds of Formula I can be made by alkylation of compound of formula 3 in the presence of a base such as K2CO3, NaH, Cs2CO3, DBU etc. with/without the presence of a catalyst such as NaI, KI, (Bu)4NI etc., and in a polar solvent such as DMF, THF, DMSO etc. using electrophiles such as L1-R3 where L1 is a leaving group. Leaving groups include but are not limited to, e.g., halogen, triflate, tosylate, mesylate (A. B. Reitz J. Med. Chem. 1994, 37, 3561 and T. Itaya Chem. Pharm. Bull. 1997, 45, 1601).

IV Pharmaceutical Compositions, Dosaging, and Modes of Administration

The present invention is directed to the clinical use of the heterocyclics, in particular, the 7,9-dihydro-purin-8-one and their related analogs of Formula I and their polymorphs, solvates, esters, tautomers, diastereomers, enantiomers, pharmaceutically acceptable salts and prodrugs thereof, for use in treatment or prevention of diseases that are HSP90-dependent. Examples of such diseases include disorders such as inflammatory diseases, infections, autoimmune disorders, stroke, ischemia, cardiac disorder, neurological disorders, fibrogenetic disorders, proliferative disorders, tumors, leukemias, neoplasms, cancers, carcinomas, metabolic diseases, and malignant disease. The fibrogenetic disorders include but are not limited to scleroderma, polymyositis, systemic lupus, rheumatoid arthritis, liver cirrhosis, keloid formation, interstitial nephritis and pulmonary fibrosis.

The present invention features pharmaceutical compositions comprising the compound of Formula I or a polymorph, solvate, ester, tautomer, enantiomer, diastereomer, pharmaceutically acceptable salt thereof, or prodrug thereof, of any of the preceding aspects and embodiments and one or more pharmaceutical excipients.

Those of ordinary skill in the art are familiar with formulation and administration techniques that can be employed with the compounds and methods of the invention, e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

The compounds utilized in the methods of the instant invention may be administered either alone or in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

For example, the therapeutic or pharmaceutical compositions of the invention can be administered locally to the area in need of treatment. This may be achieved by, for example, but not limited to, local infusion during surgery, topical application, e.g., cream, ointment, injection, catheter, or implant, said implant made, e.g., out of a porous, non-porous, or gelatinous material, including membranes, such as silastic membranes, or fibers. The administration can also be by direct injection at the site (or former site) of a tumor or neoplastic or pre-neoplastic tissue.

Still further, the compounds or compositions of the invention can be delivered in a vesicle, e.g., a liposome (see, for example, Langer, Science 1990, 249, 1527-1533; Treat et al., Liposomes in the Therapy of Infectious Disease and Cancer, Lopez-Bernstein and Fidler, Ed., Liss, N.Y., pp. 353-365, 1989).

The compounds and pharmaceutical compositions used in the methods of the present invention can also be delivered in a controlled release system. In one embodiment, a pump may be used (see, Sefton, 1987, CRC Crit. Ref. Biomed. Eng. 14:201; Buchwald et al. Surgery, 1980 88, 507; Saudek et al. N. Engl. J. Med. 1989, 321, (574). Additionally, a controlled release system can be placed in proximity of the therapeutic target. (See, Goodson, Medical Applications of Controlled Release, 1984, Vol. 2, pp. 115-138).

The pharmaceutical compositions used in the methods of the instant invention can also contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions, and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium croscarmellose, corn starch, or alginic acid; binding agents, for example starch, gelatin, polyvinyl-pyrrolidone or acacia, and lubricating agents, for example, magnesium stearate, stearic acid or talc. The tablets may be un-coated or coated by known techniques to mask the taste of the drug or delay disintegration and absorption in the gastrointestinal tract and thereby provide a sustained action over a longer period. For example, a water soluble taste masking material such as hydroxypropylmethylcellulose or hydroxypropylcellulose, or a time delay material such as ethyl cellulose, or cellulose acetate butyrate may be employed as appropriate.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisole or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The compounds and pharmaceutical compositions used in the methods of the instant invention may also be in the form of an oil-in-water emulsion. The oily phase may be a vegetable oil, for example olive oil or arachis oil, or a mineral oil, for example liquid paraffin, or mixtures of these. Suitable emulsifying agents may be naturally-occurring phosphatides, for example soybean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening agents, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solution. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution may then be introduced into a water and glycerol mixture and processed to form a microemulsion.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention used in the methods of the present invention may also be administered in the form of suppositories for rectal administration of the drug. These compositions can be prepared by mixing the inhibitors with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing a compound or composition of the invention can be used. As used herein, topical application can include mouth washes and gargles.

The compounds used in the methods of the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The methods, compounds and compositions of the instant invention may also be used in conjunction with other well known therapeutic agents that are selected for their particular usefulness against the condition that is being treated. For example, the instant compounds may be useful in combination with known anti-cancer and cytotoxic agents. Further, the instant methods and compounds may also be useful in combination with other inhibitors of parts of the signaling pathway that links cell surface growth factor receptors to nuclear signals initiating cellular proliferation.

The methods of the present invention may also be useful with other agents that inhibit angiogenesis and thereby inhibit the growth and invasiveness of tumor cells, including, but not limited to VEGF receptor inhibitors, including ribozymes and antisense targeted to VEGF receptors, angiostatin and endostatin.

Examples of antineoplastic agents that can be used in combination with the compounds and methods of the present invention include, in general, and as appropriate, alkylating agents, anti-metabolites, epidophyllotoxins, an antineoplastic enzyme, a topoisomerase inhibitor, procarbazine, mitoxantrone, platinum coordination complexes, biological response modifiers and growth inhibitors, hormonal/anti-hormonal therapeutic agents and haematopoietic growth factors. Exemplary classes of antineoplastic include the anthracyclines, vinca drugs, mitomycins, bleomycins, cytotoxic nucleosides, epothilones, discodermolide, pteridines, diynenes and podophyllotoxins. Particularly useful members of those classes include, for example, caminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, carboplatin, cyclophosphamide, bleomycin, gemcitabine, ifosfamide, melphalan, hexamethyl melamine, thiotepa, cytarabine, edatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

When a compound or composition of the invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for cancer, for example, breast cancer. Administration typically occurs in an amount of between about 0.01 mg/kg of body weight to about 100 mg/kg of body weight per day (administered in single or divided doses), more preferably at least about 0.1 mg/kg of body weight per day. A particular therapeutic dosage can include, e.g., from about 0.01 mg to about 1000 mg of compound, and preferably includes, e.g., from about 1 mg to about 1000 mg. The quantity of active compound in a unit dose of preparation may be varied or adjusted from about 0.1 mg to 1000 mg, preferably from about 1 mg to 300 mg, more preferably 10 mg to 200 mg, according to the particular application. The amount administered will vary depending on the particular IC50 value of the compound used and the judgment of the attending clinician taking into consideration factors such as health, weight, and age. In combinational applications in which the compound is not the sole active ingredient, it may be possible to administer lesser amounts of compound and still have therapeutic or prophylactic effect.

Preferably, the pharmaceutical preparation is in unit dosage form. In such form, the preparation is subdivided into unit doses containing appropriate quantities of the active component, e.g., an effective amount to achieve the desired purpose.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The amount and frequency of administration of the compounds and compositions of the present invention used in the methods of the present invention, and if applicable other chemotherapeutic agents and/or radiation therapy, will be regulated according to the judgment of the attending clinician (physician) considering such factors as age, condition and size of the patient as well as severity of the disease being treated.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the patient, and in view of the observed responses of the disease to the administered therapeutic agents.

Also, in general, the compounds of the invention need not be administered in the same pharmaceutical composition as a chemotherapeutic agent, and may, because of different physical and chemical characteristics, be administered by a different route. For example, the compounds/compositions may be administered orally to generate and maintain good blood levels thereof, while the chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of compound (and where appropriate, chemotherapeutic agent and/or radiation) will depend upon the diagnosis of the attending physicians and their judgment of the condition of the patient and the appropriate treatment protocol.

The compounds/compositions of the invention (and where appropriate chemotherapeutic agent and/or radiation) may be administered concurrently (e.g., simultaneously, essentially simultaneously or within the same treatment protocol) or sequentially, depending upon the nature of the proliferative disease, the condition of the patient, and the actual choice of chemotherapeutic agent and/or radiation to be administered in conjunction (i.e., within a single treatment protocol) with the compound/composition.

In combinational applications and uses, the compound/composition and the chemotherapeutic agent and/or radiation need not be administered simultaneously or essentially simultaneously, and the initial order of administration of the compound/composition, and the chemotherapeutic agent and/or radiation, may not be important. Thus, the compounds/compositions of the invention may be administered first followed by the administration of the chemotherapeutic agent and/or radiation; or the chemotherapeutic agent and/or radiation may be administered first followed by the administration of the compounds/compositions of the invention. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the chemotherapeutic agent and/or radiation may be administered first, especially if it is a cytotoxic agent, and then the treatment continued with the administration of the compounds/compositions of the invention followed, where determined advantageous, by the administration of the chemotherapeutic agent and/or radiation, and so on until the treatment protocol is complete.

Thus, in accordance with experience and knowledge, the practicing physician can modify each protocol for the administration of a compound/composition for treatment according to the individual patient's needs, as the treatment proceeds. The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

V. Assays for Determining HSP90 Binding and Downstream Effect

A variety of in vitro and in vivo assays are available to test the effect of the compounds of the invention on HSP90. HSP90 competitive binding assays and functional assays can be performed as known in the art by substituting in the compounds of the invention. Chiosis et al. Chemistry & Biology 2001, 8, 289-299, describe some of the known ways in which this can be done. For example, competition binding assays using, e.g., geldanamycin or 17-AAG as a competitive binding inhibitor of HSP90 can be used to determine relative HSP90 affinity of the compounds of the invention by immobilizing the compound of interest or other competitive inhibitor on a gel or solid matrix, preincubating HSP90 with the other inhibitor, passing the preincubated mix over the gel or matrix, and then measuring the amount of HSP90 that retains or does not retain on the gel or matrix.

Downstream effects can also be evaluated based on the known effect of HSP90 inhibition on function and stability of various steroid receptors and signaling proteins including, e.g., Raf1 and HER2. Compounds of the present invention induce dose-dependent degradation of these molecules, which can be measured using standard techniques. Inhibition of HSP90 also results in up-regulation of HSP90 and related chaperone proteins that can similarly be measured. Antiproliferative activity on various cancer cell lines can also be measured, as can morphological and functional differentiation related to HSP90 inhibition.

Many different types of methods are known in the art for determining protein concentrations and measuring or predicting the level of proteins within cells and in fluid samples. Indirect techniques include nucleic acid hybridization and amplification using, e.g., polymerase chain reaction (PCR). These techniques are known to the person of skill and are discussed, e.g., in Sambrook, Fritsch & Maniatis Molecular Cloning: A Laboratory Manual, 2nd ed., Cold Spring Harbor Laboratory, Cold Spring Harbor, N.Y., 1989; Ausubel, et al. Current Protocols in Molecular Biology, John Wiley & Sons, NY, 1994, and, as specifically applied to the quantification, detection, and relative activity of HER2/Neu in patient samples, e.g., in U.S. Pat. Nos. 4,699,877, 4,918,162, 4,968,603, and 5,846,749. A brief discussion of two generic techniques that can be used follows.

The determination of whether cells overexpress or contain elevated levels of HER2 can be determined using well known antibody techniques such as immunoblotting, radioimmunoassays, western blotting, immunoprecipitation, enzyme-linked immunosorbant assays (ELISA), and derivative techniques that make use of antibodies directed against HER2. As an example, HER2 expression in breast cancer cells can be determined with the use of an immunohistochemical assay, such as the Dako Hercep™ test (Dako Corp., Carpinteria, Calif.). The Hercep™ test is an antibody staining assay designed to detect HER2 overexpression in tumor tissue specimens. This particular assay grades HER2 expression into four levels: 0, 1, 2, and 3, with level 3 representing the highest level of HER2 expression. Accurate quantitation can be enhanced by employing an Automated Cellular Imaging System (ACIS) as described, e.g., by Press, M. et al. Modern Pathology 2000, 13, 225A.

Antibodies, polyclonal or monoclonal, can be purchased from a variety of commercial suppliers, or may be manufactured using well-known methods, e.g., as described in Harlow et al. Antibodies: A Laboratory Manual, 2nd ed; Cold Spring Harbor Laboratory, Cold Spring Harbor, N.Y., 1988.

HER2 overexpression can also be determined at the nucleic acid level since there is a reported high correlation between overexpression of the HER2 protein and amplification of the gene that codes for it. One way to test this is by using RT-PCR. The genomic and cDNA sequences for HER2 are known. Specific DNA primers can be generated using standard, well-known techniques, and can then be used to amplify template already present in the cell. An example of this is described in Kurokawa, H. et al. Cancer Res. 2000, 60, 5887-5894. PCR can be standardized such that quantitative differences are observed as between normal and abnormal cells, e.g., cancerous and noncancerous cells. Well known methods employing, e.g., densitometry, can be used to quantitate and/or compare nucleic acid levels amplified using PCR.

Similarly, fluorescent in situ hybridization (FISH) assays and other assays can be used, e.g., Northern and/or Southern blotting. These rely on nucleic acid hybridization between the HER2 gene or mRNA and a corresponding nucleic acid probe that can be designed in the same or a similar way as for PCR primers, above. See, e.g., Mitchell M S, and Press M. F. Oncol., Suppl. 1999, 12, 108-116. For FISH, this nucleic acid probe can be conjugated to a fluorescent molecule, e.g., fluorescein and/or rhodamine, that preferably does not interfere with hybridization, and which fluorescence can later be measured following hybridization. See, e.g., Kurokawa, H et al, Cancer Res. 2000, 60, 5887-5894 (describing a specific nucleic acid probe having sequence 5′-FAM-Nucleic Acid-TAMRA-p-3′ sequence). ACIS-based approaches as described above can be employed to make the assay more quantitative (de la Torre-Bueno, J., et al. Modern Pathology 2000, 13, 221A).

Immuno and nucleic acid detection can also be directed against proteins other than HSP90 and HER2, which proteins are nevertheless affected in response to HSP90 inhibition.

The following examples are offered by way of illustration only and are not intended to be limiting of the full scope and spirit of the invention.

### EXAMPLES

I. Materials and Methods

The chemical reagents used to create the novel products of the invention below are all available commercially, e.g., from Aldrich Chemical Co., Milwaukee, Wis., USA. Otherwise their preparation is facile and known to one of ordinary skill in the art, or it is referenced or described herein.

The final compounds were usually purified by preparative TLC (silica gel 60 Å, Whatman Partisil PK6F) or flash chromatography (silica gel 60 Å, EMD Chemicals) using EtOAc/hexane or MeOH/CH2Cl2 as eluents. Rf's were measured using silica gel TLC plates (silica gel 60 Å, EMD Chemicals). Analytical HPLC chromatograms were obtained using a C18 column (Agilent Zorbax 300SB-C18; 5 microns; 4.6 mm×150 mm). A gradient was applied between solvent A (0.1% TFA in H2O) and solvent B (0.5% TFA in CH3CN) increasing the proportion of A linearly from 5% (t=0) to 100% (t=−7.00 nm in), with a constant flow rate of 1 mL/min. The samples were diluted to typically 0.1-1 mg/mL in MeOH or CH3CN and the injection volumes were typically 10 □L. The column was not heated, and UV detection was effected at 254 nm. ¹H-NMR spectra were recorded on a Bruker Avance 400 MHz spectrometer.

The chemical names were generated using the Beilstein Autonom 2.1 software.

II. General Procedures

General procedures to prepare and manipulate the 7,9-dihydro-purin-8-one

### General Procedure 1.1

### Preparation of 4-alkylamino-6-chloro-2,5-diamine-pyrimidine

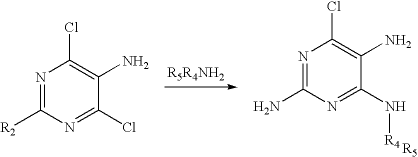

A suspension of 4,6-dichloro-pyrimidine-2,5-diamine (31.4 mmol), diisopropylethylamine (175 mmol), and R⁵R⁴—NH₂ (29.5 mmol) in n-BuOH (300 ml), was heated to 115° C. for 6 hours. After removal of the solvent, EtOAc was added to the residue, and it washed with water, brine and dried with Na2SO4 and purified by flash chromatography to give the 4-Alkylamino-6-chloro-2,5-diamine-pyrimidine.

### General Procedure 1.2

### Preparation of 2-amino-6-chloro-9-alkyl-7H-purin-8(9H)-one

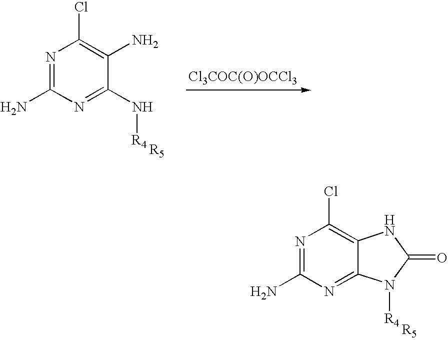

To a solution of 4-alkylamino-6-chloro-2,5-diamine-pyrimidine (15.9 mmol) in THF (100 ml) at 5° C. was added a solution of triphosgene (5.3 mmol) in THF (30 ml) over 20 minutes and stirred further for 30 minutes. After removal of the solvent, CHCl₃ was added to the residue, and it washed with water, brine, dried with Na₂SO₄, and concentrated under reduce pressure. Finally, the residue washed twice with hot EtOAc to give the 2-amino-6-chloro-9-alkyl-7H-purin-8(9H)-one.

### General procedure 1.3

### Preparation of 2-amino-6-chloro-7-alkyl-9-alkyl-7H-purin-8(9H)-one

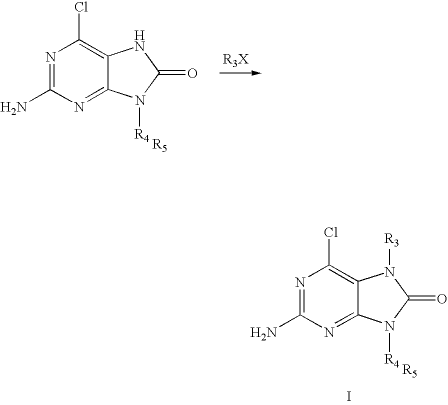

A suspension of 2-amino-6-chloro-9-alkyl-7H-purin-8(9H)-one (1 mmol), R₃X (1.1 mmol) and Cs₂CO₃ in DMF was heated to 70° C. for 2 hours. After removal of the solvent, EtOAc was added to the residue, and it washed with water, brine and dried with Na₂SO₄ and purified by flash chromatography to give 2-amino-6-chloro-7-alkyl-9-alkyl-7H-purin-8(9H)-one.

### General procedure 1.4

### Preparation of 2-amino-6-chloro-7-(2-dialkylamino-ethyl)-9-(alkyl)-7H-purin-8(9H)-one

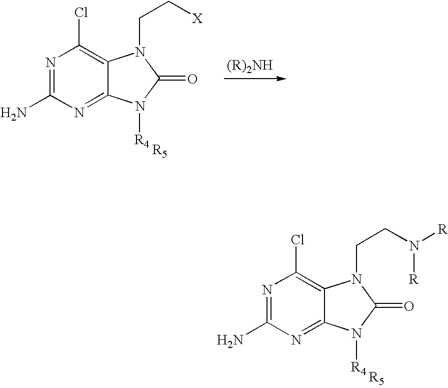

A solution of 2-Amino-7-(2-bromo-ethyl)-6-chloro-9-(alkyl)-7,9-dihydro-purin-8-one (1 mmol) Cs₂CO₃ (3 mmol) and dialkylamine (5 mmol) in DMF was heated at 70° C. for 4 hours whereupon it was extracted with EtOAc/water, brine, dried with Na₂SO₄, and purified by flash chromatography to give 2-amino-6-chloro-7-(2-dialkylamino-ethyl)-9-alkyl-7H-purin-8(9H)-one.

### General procedure 1.5

### Preparation of 7-Acetyl-2-amino-6-chloro-9-alkyl-7,9-dihydro-purin-8-one

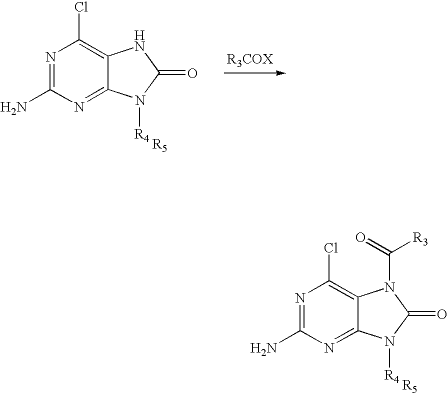

A solution of 2-Amino-6-chloro-9-(4-methoxy-3,5-dimethyl-pyridin-2-ylmethyl)-7,9-dihydro-purin-8-one (1 mmol), DBU (1 mmol) and acetic anhydride (1 mmol) in DMF was stirred at room temperature for 1 h whereupon it was partitioned between EtOAc and water, washed with brine, and purified by flash chromatography to give the 7-Acetyl-2-amino-6-chloro-9-(4-methoxy-3,5-dimethyl-pyridin-2-ylmethyl)-7,9-dihydro-purin-8-one.

### General procedure 1.6

### 2-((2-amino-6-chloro-7-alkyl-8-oxo-7H-purin-9(8H)-yl)methyl)-4-halo-3,5-dimethylpyridine 1-oxide

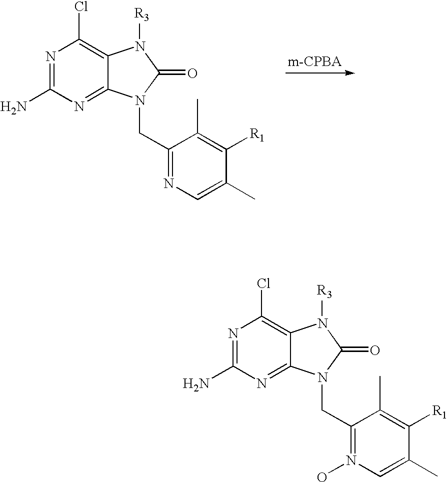

A solution of 2-amino-6-chloro-7-(alkyl)-9-((4-halo-3,5-dimethylpyridin-2-yl)methyl)-7H-purin-8(9H)-one (1 mmol) and 3-chloroperoxybenzoic acid (5 mmol) in DCM was stirred at room temperature for 1 h whereupon it was washed with NaOH 1N, water, brine, dried with Na₂SO₄ and purified by flash chromatography to give the 2-((2-amino-6-chloro-7-(2-(diisopropylamino)ethyl)-8-oxo-7H-purin-9(8H)-yl)methyl)-4-iodo-3,5-dimethylpyridine 1-oxide.

General Procedures to Manipulate the Pyridine Ring

### General procedure 2.1

### Preparation of 2-(1,3-dioxo-1,3-dihydro-isoindol-2-ylmethyl)-pyridine

A suspension of 2-chloro-methyl-pyridine derivative (1.2 mmol), phtalimide (1 mmol), Cs₂CO₃ (2.2 eq) and NaI (0.5 mmol) in DMF was heated to 100° C. 20 hours. It was then poured into water, extracted from EtOAc, dried with Na₂SO₄ and purified by flash chromatography to give the 2-(1,3-Dioxo-1,3-dihydro-isoindol-2-ylmethyl)-pyridine.

### General procedure 2.2

### Preparation of 2-(1,3-dioxo-1,3-dihydro-isoindol-2-ylmethyl)-4-hydroxy-pyridine

To a solution of 2-(1,3-dioxo-1,3-dihydro-isoindol-2-ylmethyl)-4-methoxy-pyridine (1 mmol) in THF/EtOH was added HCl conc (2 mmol) and it was concentrated. The residue was dissolved in DMF and NaBr (1 mmol) was added whereupon it was heated to 120° C. for 2 h. It was then poured into water, extracted from EtOAc, dried with Na₂SO₄ to give the 2-(1,3-Dioxo-1,3-dihydro-isoindol-2-ylmethyl)-4-hydroxy-pyridine.

### General procedure 2.3

### Preparation of 2-(1,3-dioxo-1,3-dihydro-isoindol-2-ylmethyl)-4-trifluoromethanesulfonyloxy-pyridine

To a solution of 2-(1,3-dioxo-1,3-dihydro-isoindol-2-ylmethyl)-4-hydroxy-pyridine (1 mmol) and (i-Pr)₂NEt (1.2 mmol) in CH₂Cl₂ was added dropwise at 0° C. trifluomethane sulfonic anhydride (1.2 mmol). It was then diluted with hexane, washed with water, dried with Na₂SO₄ and purified by flash chromatography to give the 2-(1,3-dioxo-1,3-dihydro-isoindol-2-ylmethyl)-4-trifluoromethanesulfonyloxy-pyridine.

### General procedure 2.4

### Preparation of 2-(1,3-dioxo-1,3-dihydro-isoindol-2-ylmethyl)-4-halo-pyridine

To a solution of 2-(1,3-dioxo-1,3-dihydro-isoindol-2-ylmethyl)-4-trifluoromethanesulfonyloxy-pyridine (1 mmol) in THF/EtOH was added HX conc. (2 mmol) and it was concentrated. The residue was dissolved in DMF and NaX (2 mmol) was added whereupon it was heated to 110° C. for 2 h. It was then poured into water, extracted from EtOAc, dried with Na₂SO₄ to give the 2-(1,3-Dioxo-1,3-dihydro-isoindol-2-ylmethyl)-4-halo-pyridine.

### General procedure 2.5

### Preparation of 2-(amino-methyl)-4-halo-pyridine

To a solution of 2-(1,3-Dioxo-1,3-dihydro-isoindol-2-ylmethyl)-4-halo-pyridine (1 mmol) in EtOH was added hydrazine (5 mmol) and it was heated at reflux for 2 h whereupon it was cooled, filtered, concentrated and washed with CH₂Cl₂ to give the 2-(amino-methyl)-4-halo-pyridine.

Preparation of Intermediates

### Example 1

### 6-chloro-N4-((4-methoxy-3,5-dimethylpyridin-2-yl)methyl)pyridine-2,4,5-triamine

The title compound was prepared by condensation between 4,6-dichloro-pyrimidine-2,5-diamine and 2-aminomethyl-3,5-dimethyl-4-methoxy-pyridine according to the general procedure 1.1 HPLC 3.52 min. ¹H-NMR (CDCl₃): δ 8.24 (s, 1H), 7.12 (br s, 1H), 4.61 (s, 2H), 4.555 (d, 2H), 3.80 (s, 3H), 3.0 (s, 2H), 2.29 (s, 3H), 2.27 (s, 3H).

### Example 2

### 2-amino-6-chloro-9-((4-methoxy-3,5-dimethylpyridin-2-yl)methyl)-7H-purin-8(9H)-one

The title compound was prepared by condensation between 6-chloro-N4-((4-methoxy-3,5-dimethylpyridin-2-yl)methyl)pyrimidine-2,4,5-triamine and triphosgene according to the general procedure 1.2. HPLC Rt: 4.42 min. ¹H-NMR (DMSO): δ 10.07 (s, 1H), 8.01 (s, 1H), 5.85 (s, 2H), 5.04 (s, 2H), 3.83 (s, 3H), 2.29 (s, 3H), 2.22 (s, 3H).

### Example 3

### N4-((4-bromo-3,5-dimethylpyridin-2-yl)methyl)-6-chloropyrimidine-2,4,5-triamine

The title compound was prepared by condensation between 4,6-dichloro-pyrimidine-2,5-diamine and 2-aminomethyl-4-bromo-3,5-dimethyl-pyridine according to the general procedure 1.1. HPLC Rt: 3.86 min. ¹H-NMR (DMSO) δ 8.34 (s, 1H), 7.07 (t, 1H), 5.75 (s, 2H), 4.67 (d, 2H), 3.97 (s, 2H), 2.45 (s, 3H), 2.38 (s, 3H).

### Example 4

### 2-amino-9-((4-bromo-3,5-dimethylpyridin-2-yl)methyl)-6-chloro-7H-purin-8(9H)-one

The title compound was prepared by condensation between N4-((4-bromo-3,5-dimethylpyridin-2-yl)methyl)-6-chloropyrimidine-2,4,5-triamine and triphosgene according to the general procedure 1.2. HPLC Rt: 4.84 nm in. ¹H-NMR (DMSO) δ 11.29 (s, 1H), 8.10 (s, 1H), 6.52 (s, 2H), 5.03 (s, 2H), 2.46 (s, 3H), 2.28 (s, 3H).

### Example 5

### 6-chloro-N4-((4-iodo-3,5-dimethylpyridin-2-yl)methyl)pyrimidine-2,4,5-triamine

The title compound was prepared by condensation between 4,6-dichloro-pyrimidine-2,5-diamine and 2-aminomethyl-3,5-dimethyl-4-iodo-pyridine according to the general procedure 1.1. HPLC Rt: 4.10 min. ¹H-NMR (CDCl₃) δ 8.14 (s, 1H), 7.05 (s, 1H), 4.60 (s, 2H), 4.59 (s, 2H), 4.57 (s, 2H), 3.48 (s, 3H), 2.42 (s, 3H).

### Example 6

### 2-amino-6-chloro-9-((4-iodo-3,5-dimethylpyridin-2-yl)methyl)-7H-purin-8(9H)-one

The title compound was prepared by condensation between N4-((4-iodo-3,5-dimethylpyridin-2-yl)methyl)-6-chloropyrimidine-2,4,5-triamine and triphosgene according to the general procedure 1.2. HPLC Rt: 4.715 min. ¹H-NMR (DMSO) δ 11.30 (s, 1H), 8.01 (s, 1H), 6.53 (s, 2H), 5.03 (s, 2H), 2.51 (s, 3H), 2.32 (s, 3H).

### Example 7

### N4-(2-bromo-3,4,5-trimethoxybenzyl)-6-chloropyrimidine-2,4,5-triamine

The title compound was prepared by condensation between 4,6-dichloro-pyrimidine-2,5-diamine and 2-bromo-3,4,5-trimethoxybenzylamine according to the general procedure 1.1. HPLC Rt: 4.04 min. ¹H-NMR (CDCl₃) δ 6.82 (s, 1H), 5.80 (t, 1H), 4.67 (d, 2H), 4.63 (s, 2H), 4.60 (s, 2H), 3.90 (s, 3H), 3.87 (s, 3H), 3.83 (s, 3H).

### Example 8

### 2-amino-9-(2-bromo-3,4,5-trimethoxybenzyl)-6-chloro-7H-purin-8(9H)-one

The title compound was prepared by condensation N4-(2-bromo-3,4,5-trimethoxybenzyl)-6-chloropyrimidine-2,4,5-triamine and triphosgene according to the general procedure 1.2. HPLC Rt: 5.46 min. ¹H-NMR (DMSO) δ 11.41 (s, 1H), 6.64 (s, 2H), 6.47 (s, 1H), 4.83 (s, 2H), 3.80 (s, 3H), 3.75 (s, 3H), 3.65 (s, 3H).

### Example 9

### 6-chloro-N4-((5-methoxy-4,6-dimethylpyridin-3-yl)methyl)pyrimidine-2,4,5-triamine

The title compound was prepared by condensation between 4,6-dichloro-pyrimidine-2,5-diamine and 3-aminomethyl-4,6-dimethyl-5-methoxy-pyridine according to the general procedure 1.1. HPLC Rt: 3.36 min. ¹H-NMR (DMSO) δ 8.14 (s, 1H), 6.83 (t, 1H), 5.66 (s, 2H), 4.48 (d, 2H), 3.94 (s, 2H), 3.66 (s, 3H), 2.38 (s, 3H), 2.23 (s, 3H).

### Example 10

### 2-amino-6-chloro-9-((5-methoxy-4,6-dimethylpyridin-3-yl)methyl)-7H-purin-8(9H)-one

The title compound was prepared by 6-chloro-N4-((5-methoxy-4,6-dimethylpyridin-3-yl)methyl)pyrimidine-2,4,5-triamine and triphosgene according to the general procedure 1.2. HPLC 3.77 min. ¹H-NMR (DMSO): δ 11.37 (s, 1H), 7.98 (s, 1H), 6.62 (s, 2H), 4.85 (s, 2H), 3.66 (s, 3H), 2.37 (s, 3H), 2.32 (s, 3H).

### Example 11

### 2-(amino-methyl)-4-halo-pyridine

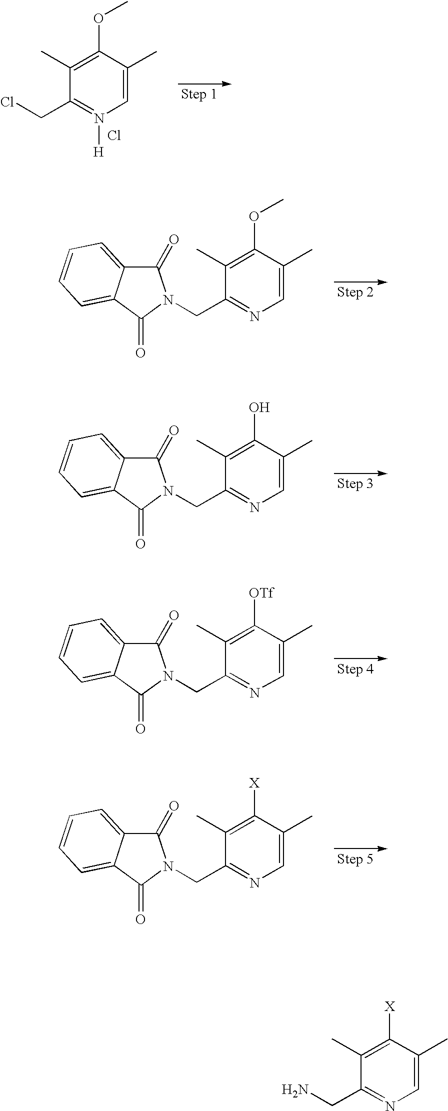

### Step 1

### 2-(1,3-Dioxo-1,3-dihydro-isoindol-2-ylmethyl)-3, S-dimethyl-4-methoxy-pyridine

The title compound was prepared by alkylation of phtalimide with the HCl salt of 2-chloromethyl-4-methoxy-3,5-dimethyl-pyridine according to the general procedure 2.1. HPLC Rt: 4.72 min. ¹H-NMR (CDCl₃): δ 8.05 (s, 1H), 7.89-7.87 (m, 2H), 7.73-7.70 (m, 2H), 4.92 (s, 2H), 3.75 (s, 3H), 2.32 (s, 3H), 2.17 (s, 3H).

### Step 2

### 2-(1,3-Dioxo-1,3-dihydro-isoindol-2-ylmethyl)-3,5-dimethyl-4-hydroxy-pyridine

The title compound was prepared by demethylation of the HCl salt of 2-(1,3-dioxo-1,3-dihydro-isoindol-2-ylmethyl)-pyridine according to the general procedure 2.2. HPLC Rt: 4.41 min. ¹H-NMR (CDCl₃): δ 7.87-7.84 (m, 2H), 7.77-7.74 (m, 2H), 7.45 (s, 1H), 4.88 (s, 2H), 2.25 (s, 3H), 2.03 (s, 3H).

### Step 3

### 2-(1,3-Dioxo-1,3-dihydro-isoindol-2-ylmethyl)-3,5-dimethyl-4-trifluoromethanesulfonyloxy-pyridine

The title compound was prepared by treating 2-(1,3-Dioxo-1,3-dihydro-isoindol-2-ylmethyl)-3,5-dimethyl-4-hydroxy-pyridine with trifluoromethane sulfonic anhydride according to the general procedure 2.3. HPLC Rt: 7.38 min. ¹H-NMR (CDCl₃): δ 8.20 (s, 1H), 7.91-7.89 (m, 2H), 7.78-7.74 (m, 2H), 4.98 (s, 2H), 2.44 (s, 3H), 2.31 (s, 3H).

### Step 4

### 2-(1,3-Dioxo-1,3-dihydro-isoindol-2-ylmethyl)-3,5-dimethyl-4-_iodo-pyridine

The title compound was prepared by treating the HI salt of 2-(1,3-Dioxo-1,3-dihydro-isoindol-2-ylmethyl)-3,5-dimethyl-4-trifluoromethanesulfonyloxy-pyridine with NaI according to the general procedure 2.4. HPLC Rt: 6.40 min. ¹H-NMR (CDCl₃): δ 8.04 (s, 1H), 7.90-7.88 (m, 2H), 7.76-7.74 (m, 2H), 5.04 (s, 2H), 2.60 (s, 3H), 2.39 (s, 3H).

### Step 4

### 2-(1,3-Dioxo-1,3-dihydro-isoindol-2-ylmethyl)-3,5-dimethyl-4-bromo-pyridine

The title compound was prepared by treating the HBr salt of 2-(1,3-Dioxo-1,3-dihydro-isoindol-2-ylmethyl)-3,5-dimethyl-4-trifluoromethanesulfonyloxy-pyridine with NaBr according to the general procedure 2.4. HPLC Rt: 6.26 min. ¹H-NMR (CDCl₃): δ 8.03 (s, 1H), 7.89-7.87 (m, 2H), 7.75-7.73 (m, 2H), 4.98 (s, 2H), 2.50 (s, 3H), 2.30 (s, 3H).

### Step 5

### 2-(amino-methyl)-4-iodo-pyridine

The title compound was prepared by treating 2-(1,3-Dioxo-1,3-dihydro-isoindol-2-ylmethyl)-3,5-dimethyl-4-iodo-pyridine with hydrazine according to the general procedure 2.5. HPLC Rt: 4.45 min. ¹H-NMR (CDCl₃): δ 8.31 (s, 1H), 3.94 (s, 2H), 2.45 (s, 3H), 2.39 (s, 3H).

### Step 5

### 2-(amino-methyl)-4-bromo-pyridine

The title compound was prepared by treating 2-(1,3-Dioxo-1,3-dihydro-isoindol-2-ylmethyl)-3,5-dimethyl-4-bromo-pyridine with hydrazine according to the general procedure 2.5. HPLC Rt: 4.32 min. ¹H-NMR (CDCl₃): δ 8.30 (s, 1H), 3.90 (s, 2H), 2.40 (s, 3H), 2.36 (s, 3H).

### Example 12

### 3-(amino-methyl)-5-methoxy-pyridine

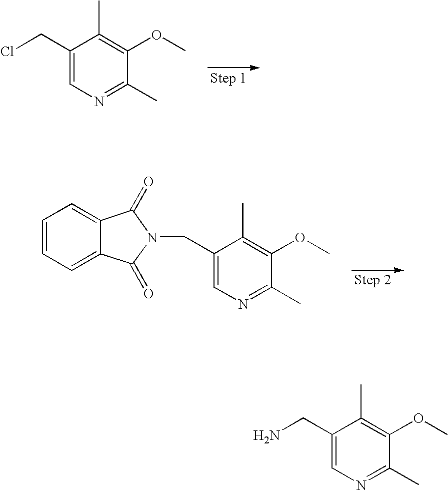

### Step 1

### 3-(1,3-Dioxo-1,3-dihydro-isoindol-2-ylmethyl)-4,6-dimethyl-5-methoxy-pyridine

The title compound was prepared by alkylation of phtalimide with the HCl salt of 3-chloromethyl-5-methoxy-4,6-dimethyl-pyridine according to the general procedure 2.1. HPLC Rt: 4.61 min. ¹H-NMR (CDCl₃): δ 8.28 (s, 1H), 7.86-7.83 (m, 2H), 7.75-7.71 (m, 2H), 4.84 (s, 2H), 3.70 (s, 3H), 2.47 (s, 3H), 2.40 (s, 3H).

### Step 2

### 3-(amino-methyl)-4,6-dimethyl-5-methoxy-pyridine

The title compound was prepared by treating 3-(1,3-Dioxo-1,3-dihydro-isoindol-2-ylmethyl)-4,6-dimethyl-5-methoxy-pyridine with hydrazine according to the general procedure 2.5. HPLC Rt: 2.11 min. ¹H-NMR (CDCl₃): δ 8.16 (s, 1H), 3.85 (s, 2H), 3.75 (s, 3H), 2.49 (s, 3H), 2.30 (s, 3H), 1.26 (br s, 2H).

Preparation of Final Compounds

### Example 13

### 2-Amino-6-chloro-9-(4-methoxy-3,5-dimethyl-pyridin-2-ylmethyl)-7-methyl-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with methyl iodide according to the general procedure 1.3. HPLC Rt: 4.54 min. ¹H-NMR (CDCl₃) δ 8.08 (s, 1H), 5.08 (s, 2H), 4.86 (s, 2H), 3.77 (s, 3H), 3.63 (s, 3H), 2.34 (s, 3H), 2.21 (s, 3H).

### Example 14

### 2-amino-6-chloro-7-ethyl-9-((4-methoxy-3,5-dimethylpyridin-2-yl)methyl)-7H-purin-8(9H)-one

The title compound was obtained by alkylation of 2-Amino-6-chloro-9-(4-methoxy-3,5-dimethyl-pyridin-2-ylmethyl)-7,9-dihydro-purin-8-one with ethyl iodide according to the general procedure 1.3. HPLC Rt: 4.60 min. ¹H-NMR (CDCl₃) δ 8.18 (s, 1H), 5.11 (s, 2H), 5.01 (s, 2H), 4.10 (q, 2H), 3.80 (s, 3H), 2.33 (s, 3H), 2.23 (s, 3H), 1.34 (t, 3H).

### Example 15

### 2-amino-6-chloro-7-isopropyl-9-((4-methoxy-3,5-dimethylpyridin-2-yl)methyl)-7H-purin-8(9H)-one

The title compound was obtained by alkylation of 2-Amino-6-chloro-9-(4-methoxy-3,5-dimethyl-pyridin-2-ylmethyl)-7,9-dihydro-purin-8-one with isopropyl iodide according to the general procedure 1.3. HPLC Rt: 4.98 min. ¹H-NMR (CDCl₃) δ 8.07 (s, 1H), 5.06 (s, 2H), 5.03 (h, 1H), 4.85 (s, 2H), 3.76 (s, 3H), 2.32 (s, 3H), 2.21 (s, 3H), 1.61 (d, 6H).

### Example 16

### 2-Amino-7-butyl-6-chloro-9-(4-methoxy-3,5-dimethyl-pyridin-2-ylmethyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-iodo-butane according to the general procedure 1.3. HPLC Rt: 5.35 min. ¹H-NMR (CDCl₃) δ 8.02 (s, 1H), 5.07 (s, 2H), 4.90 (s, 2H), 4.06 (t, 2H), 3.76 (s, 3H), 2.32 (s, 3H), 2.20 (s, 3H), 1.79-1.74 (m, 2H), 1.45-1.39 (m, 2H), 0.97 (t, 3H).

### Example 17

### 2-Amino-6-chloro-7-heptyl-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromo-heptane according to the general procedure 1.3. HPLC Rt: 6.38 min. ¹H-NMR (CDCl₃) δ 8.03 (s, 1H), 5.06 (s, 2H), 4.83 (s, 2H), 4.03 (t, 2H), 3.75 (s, 3H), 2.31 (s, 3H), 2.18 (s, 3H), 1.77-1.73 (m, 2H) 1.35-1.25 (m, 8H), 0.87 (s, 3H).

### Example 18

### 2-Amino-7-benzyl-6-chloro-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with bromomethyl-benzene according to the general procedure 1.3. HPLC Rt: 5.40 min. ¹H-NMR (CDCl₃) δ 8.02 (s, 1H), 7.27 (m, 5H), 5.29 (s, 2H), 5.13 (s, 2H), 4.87 (s, 2H), 3.77 (s, 3H), 2.32 (s, 3H), 2.19 (s, 3H)

### Example 19

### 2-Amino-6-chloro-9-(4-methoxy-3,5-dimethyl-pyridin-2-ylmethyl)-7-pentyl-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-Chloro-pentane according to the general procedure 1.3. HPLC Rt: 5.91 min. ¹H-NMR (CDCl₃) δ 8.03 (s, 1H), 5.06 (s, 2H), 4.87 (s, 2H), 4.04 (t, 2H), 3.75 (s, 3H), 2.30 (s, 3H), 2.18 (s, 3H), 1.76 (m, 2H), 1.35 (m, 4H), 0.90 (t, 3H).

### Example 20

### 2-Amino-6-chloro-9-(4-methoxy-3,5-dimethyl-pyridin-2-ylmethyl)-7-(4-methyl-pent-3-enyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-Chloro-4-methyl-pent-3-ene according to the general procedure 1.3. HPLC Rt: 5.76 min. ¹H-NMR (CDCl₃) δ 8.05 (s, 1H), 5.16 (m, 1H), 5.06 (s, 2H), 4.83 (s, 2H), 4.03 (m, 2H), 3.75 (s, 3H), 2.45 (m, 2H), 2.32 (s, 3H), 2.18 (s, 3H), 1.67 (s, 3H), 1.59 (s, 3H).

### Example 21

### 2-Amino-6-chloro-7-(2-dimethylamino-ethyl)-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with (2-chloro-ethyl)-dimethyl-amine according to the general procedure 1.3. HPLC Rt: 3.94 min. ¹H-NMR (CDCl₃) δ 8.03 (s, 1H), 5.03 (s, 2H), 4.98 (s, 2H), 4.15 (t, 2H), 3.73 (s, 3H), 2.66 (t, 2H), 2.30 (s, 6H), 2.28 (s, 3H), 2.17 (s, 3H).

### Example 22

### Acetic acid 2-[2-amino-6-chloro-9-(4-methoxy-3,5-dimethyl-pyridin-2-ylmethyl)-8-oxo-8,9-dihydro-purin-7-yl]-ethyl ester

The title compound was obtained by alkylation of 2-Amino-6-chloro-9-(4-methoxy-3,5-dimethyl-pyridin-2-ylmethyl)-7,9-dihydro-purin-8-one with Acetic acid 2-chloro-ethyl ester according to the general procedure 1.3. HPLC Rt: 4.42 min. ¹H-NMR (CDCl₃) δ 8.03 (s, 1H), 5.05 (s, 2H), 4.91 (s, 2H), 4.36 (m, 4H), 3.74 (s, 3H), 2.30 (s, 3H), 2.18 (s, 3H), 1.99 (s, 3H).

### Example 23

### 2-Amino-6-chloro-9-(4-methoxy-3,5-dimethyl-pyridin-2-ylmethyl)-7-(3-pyrrol-1-yl-propyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-(3-Chloro-propyl)-1H-pyrrole according to the general procedure 1.3. HPLC Rt: 5.33 min. ¹H-NMR (CDCl₃) δ 7.99 (s, 1H), 6.68 (m, 2H), 6.11 (m, 2H), 5.06 (s, 2H), 4.87 (s, 2H), 4.09 (t, 2H), 3.99 (t, 2H), 3.74 (s, 3H), 2.30 (s, 3H), 2.25 (m, 2H), 2.17 (s, 3H).

### Example 24

### 2-Amino-6-chloro-7-(3,5-dimethoxy-benzyl)-9-(4-methoxy-3,5-dimethyl-pyridin-2-ylmethyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one 1-Bromomethyl-3,5-dimethoxy-benzene according to the general procedure 1.3. HPLC Rt: 5.48 min. ¹H-NMR (CDCl₃) δ 8.08 (s, 1H), 6.46 (s, 2H), 6.35 (m, 1H), 5.20 (s, 2H) 5.10 (s, 2H), 4.87 (s, 2H), 3.75 (s, 6H), 3.74 (s, 3H), 2.30 (s, 3H), 2.18 (s, 3H).

### Example 25

### 2-Amino-6-chloro-9-(4-methoxy-3,5-dimethyl-pyridin-2-ylmethyl)-7-(2-morpholin-4-yl-ethyl)-7,9-dihydro-purin-8-one

The title compound was obtained by alkylation of morpholine with 2-Amino-7-(2-bromo-ethyl)-6-chloro-9-(4-methoxy-3,5-dimethyl-pyridin-2-ylmethyl)-7,9-dihydro-purin-8-one according to the general 1.4. HPLC Rt: 3.79 min. ¹H-NMR (CDCl₃) δ 8.05 (s, 1H), 5.06 (s, 2H), 4.82 (s, 2H), 4.20 (t, 2H), 3.76 (s, 3H), 3.65 (t, 4H), 2.71 (t, 2H), 2.54 (m, 4H), 2.31 (s, 3H), 2.20 (s, 3H).

### Example 26

### 2-Amino-6-chloro-9-(4-methoxy-3,5-dimethyl-pyridin-2-ylmethyl)-7-pent-4-ynyl-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 5-chloro-pent-1-yne according to the general procedure 1.3. HPLC Rt: 5.02 min. ¹H-NMR (CDCl₃) δ 8.03 (s, 1H), 5.05 (s, 2H), 4.88 (s, 2H), 4.15 (t, 2H), 3.74 (s, 3H), 2.32 (m, 2H), 2.30 (s, 3H), 2.17 (s, 3H), 2.01 (m, 2H), 1.98 (t, 1H).

### Example 27

### 2-Amino-6-chloro-7-(3,4-dimethoxy-pyridin-2-ylmethyl)-9-(4-methoxy-3,5-dimethyl-pyridin-2-ylmethyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 2-chloromethyl-3,4-dimethoxy-pyridine according to the general procedure 1.3. HPLC Rt: 4.34 min. ¹H-NMR (CDCl₃) δ 8.10 (s, 1H), 8.06 (d, 1H), 6.73 (d, 1H), 5.42 (s, 2H) 5.11 (s, 2H), 4.88 (s, 2H), 3.90 (s, 3H), 3.89 (s, 3H), 3.74 (s, 3H), 2.30 (s, 3H), 2.18 (s, 3H).

### Example 28

### 2-Amino-6-chloro-9-(4-methoxy-3,5-dimethyl-pyridin-2-ylmethyl)-7-(2-methyl-thiazol-4-ylmethyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 4-chloromethyl-2-methyl-thiazole according to the general procedure 1.3. HPLC Rt: 4.96 min. ¹H-NMR (CDCl₃) δ 8.10 (s, 1H), 6.85 (s, 2H), 5.42 (s, 2H), 5.11 (s, 2H), 4.88 (s, 2H), 3.74 (s, 3H), 2.68 (s, 3H), 2.30 (s, 3H), 2.18 (s, 3H).

### Example 29

### 2-Amino-6-chloro-7-(4-fluoro-2-trifluoromethyl-benzyl)-9-(4-methoxy-3,5-dimethyl-pyridin-2-ylmethyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-Bromomethyl-4-fluoro-2-trifluoromethyl-benzene according to the general procedure 1.3. HPLC Rt: 6.04 min. ¹H-NMR (CDCl₃) δ 8.04 (s, 1H), 7.53 (m, 1H), 7.41 (m, 1H), 7.20 (m, 1H), 5.41 (s, 2H) 5.13 (s, 2H), 4.88 (s, 2H), 3.76 (s, 3H), 2.31 (s, 3H), 2.20 (s, 3H).

### Example 30

### [2-Amino-6-chloro-9-(4-methoxy-3,5-dimethyl-pyridin-2-ylmethyl)-8-oxo-8,9-dihydro-purin-7-yl]-acetic acid ethyl ester

The title compound was obtained by alkylation of 2-amino-6-chloro-9-(4-methoxy-3,5-dimethyl-pyridin-2-ylmethyl)-7,9-dihydro-purin-8-one with bromo-acetic acid ethyl ester according to the general procedure 1.3. HPLC Rt: 4.83 min. ¹H-NMR (CDCl₃) δ 8.06 (s, 1H), 5.07 (s, 2H) 4.90 (s, 2H), 4.80 (s, 2H), 4.25 (q, 2H), 3.74 (s, 3H), 2.30 (s, 3H), 2.18 (s, 3H), 1.28 (t, 3H).

### Example 31

### Acetic acid 3-[2-amino-6-chloro-9-(4-methoxy-3,5-dimethyl-pyridin-2-ylmethyl)-8-oxo-8,9-dihydro-purin-7-yl]-propyl ester

The title compound was obtained by alkylation of 2-Amino-6-chloro-9-(4-methoxy-3,5-dimethyl-pyridin-2-ylmethyl)-7,9-dihydro-purin-8-one with Acetic acid 3-chloro-propyl ester according to the general procedure 1.3. HPLC Rt: 4.74 min. ¹H-NMR (CDCl₃) δ 8.01 (s, 1H), 5.97 (s, 2H), 5.03 (s, 2H), 4.15 (m, 4H), 3.74 (s, 3H), 2.28 (s, 3H), 2.16 (s, 3H), 2.10 (m, 2H), 2.00 (s, 3H).

### Example 32

### 2-Amino-6-chloro-7-(3-hydroxy-propyl)-9-(4-methoxy-3,5-dimethyl-pyridin-2-ylmethyl)-7,9-dihydro-purin-8-one

The title compound was obtained by the hydrolysis of the acetic acid 3-[2-amino-6-chloro-9-(4-methoxy-3,5-dimethyl-pyridin-2-ylmethyl)-8-oxo-8,9-dihydro-purin-7-yl]-propyl ester (1 mmol) with catalytic potassium carbonate (0.11 mmol) in MeOH at 60° C. for one hour. The solvent was removed and the residue extracted with EtOAc, dried with Na₂SO₄ and purified by column chromatography. HPLC Rt: 4.10 min. ¹H-NMR (CDCl₃) δ 8.02 (s, 1H), 5.07 (s, 2H), 4.64 (d, 2H), 4.13 (m, 2H), 3.78 (s, 3H), 3.61 (m, 2H), 2.33 (s, 3H), 2.17 (s, 3H), 1.91 (m, 2H).

### Example 33

### 2-Amino-6-chloro-9-(4-methoxy-3,5-dimethyl-pyridin-2-ylmethyl)-7-phenethyl-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with (2-chloro-ethyl)-benzene according to the general procedure 1.3. Rt: 5.54 min. ¹H-NMR (CDCl₃) δ 8.07 (s, 1H), 7.29-7.22 (m, 5H), 5.06 (s, 2H), 4.88 (m, 2H), 4.27 (m, 2H), 3.75 (s, 3H), 3.05 (m, 2H), 2.31 (s, 3H), 2.19 (s, 3H).

### Example 34

### 2-Amino-6-chloro-9-(4-methoxy-3,5-dimethyl-pyridin-2-ylmethyl)-7-prop-2-ynyl-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 3-chloro-propyne according to the general procedure 1.3. HPLC Rt: 4.62 min. ¹H-NMR (CDCl₃) δ 8.05 (s, 1H), 5.89 (s, 2H), 5.06 (s, 2H), 4.83 (d, 2H), 3.74 (s, 3H), 2.32 (s, 3H) 2.32 (s, 1H), 2.18 (s, 3H).

### Example 35

### 2-Amino-6-chloro-9-(4-methoxy-3,5-dimethyl-pyridin-2-ylmethyl)-7-pent-4-enyl-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 5-bromo-pent-1-ene according to the general procedure 1.3. HPLC Rt: 5.43 min. ¹H-NMR (CDCl₃) δ 8.03 (s, 1H), 5.82 (m, 1H), 5.06 (s, 2H), 4.99 (m, 2H), 4.87 (s, 2H), 4.06 (t, 2H), 3.74 (s, 3H), 2.31 (s, 3H), 2.18 (s, 3H), 2.14 (m, 2H), 1.18 (m, 2H).

### Example 36

### 2-Amino-6-chloro-7-hexyl-9-(4-methoxy-3,5-dimethyl-pyridin-2-ylmethyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-Bromo-hexane according to the general procedure 1.3. HPLC Rt: 6.07 min. ¹H-NMR (CDCl₃) δ 8.03 (s, 1H), 5.06 (s, 2H), 4.84 (s, 2H), 4.03 (t, 2H), 3.74 (s, 3H), 2.31 (s, 3H), 2.18 (s, 3H), 1.77-1.69 (m, 2H), 1.37-1.29 (m, 6H), 0.87 (t, 3H).

### Example 37

### 2-Amino-6-chloro-9-(4-methoxy-3,5-dimethyl-pyridin-2-ylmethyl)-7-(4-methyl-pentyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromo-4-methyl-pentane according to the general procedure 1.3. HPLC Rt: 5.99 min. ¹H-NMR (CDCl₃) δ 8.04 (s, 1H), 5.06 (s, 2H), 4.87 (s, 2H), 4.03 (t, 2H), 3.74 (s, 3H), 2.31 (s, 3H), 2.18 (s, 3H), 1.79-1.71 (m, 2H), 1.59-1.54 (m, 1H), 1.27-1.22 (m, 2H), 0.89 (t, 3H), 0.84 (t, 3H).

### Example 38

### 2-Amino-6-chloro-9-(4-methoxy-3,5-dimethyl-pyridin-2-ylmethyl)-7-(2,3,6-trifluoro-benzyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 2-bromomethyl-1,3,4-trifluoro-benzene according to the general procedure 1.3. HPLC Rt: 5.52 min. ¹H-NMR (CDCl₃) δ 8.05 (s, 1H), 7.09 (m, 1H), 6.81 (m, 1H), 5.41 (s, 2H) 5.07 (m, 2H), 4.81 (s, 2H), 3.74 (s, 3H), 2.29 (s, 3H), 2.18 (s, 3H).

### Example 39

### 2-Amino-6-chloro-9-(4-methoxy-3,5-dimethyl-pyridin-2-ylmethyl)-7-pent-2-ynyl-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-chloro-pent-2-yne according to the general procedure 1.3. HPLC Rt: 5.27 min. ¹H-NMR (CDCl₃) δ 8.06 (s, 1H), 5.06 (s, 2H), 4.91 (s, 2H), 4.78 (s, 2H), 3.74 (s, 3H), 2.31 (s, 3H), 2.18 (s, 3H), 2.17 (m, 2H), 1.10 (t, 3H).

### Example 40

### 2-Amino-7-(3-bromo-propyl)-6-chloro-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1,3-dibromo-propane according to the general procedure 1.3 using 1:3 DMF/Dibromopropane as solvent. HPLC Rt: 5.70 min. ¹H-NMR (CDCl₃) δ 8.03 (s, 1H), 5.06 (s, 2H), 4.83 (s, 2H), 4.22 (t, 2H), 3.75 (s, 3H), 3.46 (t, 2H), 2.34 (m, 2H), 2.30 (s, 3H), 2.18 (s, 3H).

### Example 41

### 4-[2-Amino-6-chloro-9-(4-methoxy-3,5-dimethyl-pyridin-2-ylmethyl)-8-oxo-8,9-dihydro-purin-7-yl]-butyronitrile

The title compound was obtained by alkylation of 2-amino-6-chloro-9-(4-methoxy-3,5-dimethyl-pyridin-2-ylmethyl)-7,9-dihydro-purin-8-one with 4-bromo-butyronitrile according to the general procedure 1.3. HPLC Rt: 4.71 min. ¹H-NMR (CDCl₃) δ 8.00 (s, 1H), 5.06 (s, 2H), 4.94 (s, 2H), 4.20 (t, 2H), 3.75 (s, 3H), 2.48 (t, 2H), 2.30 (s, 3H), 2.20 (s, 3H), 2.18 (m, 2H).

### Example 42

### 2-Amino-6-chloro-9-(4-methoxy-3,5-dimethyl-pyridin-2-ylmethyl)-7-(3-methyl-but-2-enyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromo-3-methyl-but-2-ene according to the general procedure 1.3. HPLC Rt: 5.70 min. ¹H-NMR (CDCl₃) δ 8.05 (s, 1H), 5.30 (m, 1H), 5.06 (s, 2H), 4.94 (s, 2H), 4.64 (d, 2H), 3.75 (s, 3H), 2.30 (s, 3H), 2.18 (s, 3H), 1.79 (s, 3H), 1.72 (s, 3H).

### Example 43

### 2-Amino-6-chloro-7-(2-diethylamino-ethyl)-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with (2-chloro-ethyl)-diethyl-amine according to the general procedure 1.3. HPLC Rt: 4.12 min. ¹H-NMR (CDCl₃) δ 8.05 (s, 1H), 5.07 (s, 2H), 4.84 (s, 2H), 4.14 (t, 2H), 3.75 (s, 3H), 2.78 (t, 2H), 2.59 (q, 4H), 2.31 (s, 3H), 2.19 (s, 3H), 1.01 (t, 6H).

### Example 44

### 2-Amino-6-chloro-9-(4-methoxy-3,5-dimethyl-pyridin-2-ylmethyl)-7-(2-methylsulfanyl-ethyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-Chloro-2-methylsulfanyl-ethane according to the general procedure 1.3. HPLC Rt: 5.18 min. ¹H-NMR (CDCl₃) δ 8.05 (s, 1H), 5.07 (s, 2H), 4.87 (s, 2H), 4.26 (dd, 2H), 3.75 (s, 3H), 2.85 (dd, 2H), 2.31 (s, 3H), 2.19 (s, 3H), 2.18 (s, 3H).

### Example 45

### 2-Amino-6-chloro-9-(4-methoxy-3,5-dimethyl-pyridin-2-ylmethyl)-7-propyl-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromo-propane according to the general procedure 1.3. HPLC Rt: 5.19 min. ¹H-NMR (CDCl₃) δ 8.03 (s, 1H), 5.06 (s, 2H), 4.90 (s, 2H), 4.01 (t, 2H), 3.75 (s, 3H), 2.30 (s, 3H), 2.18 (s, 3H), 1.82-1.76 (m, 2H), 0.97 (t, 3H).

### Example 46

### 2-Amino-6-chloro-9-(4-methoxy-3,5-dimethyl-pyridin-2-ylmethyl)-7-(2-methoxy-ethyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-Chloro-2-methoxy-ethane according to the general procedure 1.3. HPLC Rt: 4.46 min. ¹H-NMR (CDCl₃) δ 8.05 (s, 1H), 5.07 (s, 2H), 4.86 (s, 2H), 4.27 (t, 2H), 3.75 (s, 3H), 3.70 (t, 2H), 3.67 (s, 3H), 2.30 (s, 3H), 2.18 (s, 3H).

### Example 47

### 2-Amino-7-but-3-enyl-6-chloro-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 4-bromo-but-1-ene according to the general procedure 1.3. HPLC Rt: 5.28 min. ¹H-NMR (CDCl₃) δ 8.03 (s, 1H), 5.81 (m, 1H), 5.10-5.02 (m, 2H), 5.07 (s, 2H), 4.98 (s, 2H), 4.12 (t, 2H), 3.74 (s, 3H), 2.50 (q, 2H), 2.29 (s, 3H), 2.17 (s, 3H).

### Example 48

### 2-Amino-6-chloro-7-(3,4-dichloro-benzyl)-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1,2-dichloro-4-chloromethyl-benzene according to the general procedure 1.3. HPLC Rt: 6.31 min. ¹H-NMR (CDCl₃) δ 8.06 (s, 1H), 7.41 (m, 2H), 7.18 (m, 1H), 5.29 (s, 2H), 5.11 (s, 2H), 4.84 (s, 2H), 3.75 (s, 3H), 2.32 (s, 3H), 2.20 (s, 3H).

### Example 49

### 2-amino-6-chloro-7-(3-(diethylamino)propyl)-9-((4-methoxy-3,5-dimethylpyridin-2-yl)methyl)-7H-purin-8(9H)-one

The title compound was obtained by alkylation of diethylamine with 2-Amino-7-(2-bromo-propyl)-6-chloro-9-(4-methoxy-3,5-dimethyl-pyridin-2-ylmethyl)-7,9-dihydro-purin-8-one according to the general procedure 1.4. HPLC Rt: 3.96 min. ¹H-NMR (CDCl₃) δ 8.02 (s, 1H), 5.05 (s, 2H), 4.87 (s, 2H), 4.08 (t, 2H), 3.74 (s, 3H), 2.56 (m, 6H), 2.30 (s, 3H), 2.18 (s, 3H), 1.95 (m, 2H), 1.02 (t, 3H), 1.01 (t, 3H).

### Example 50

### 2-Amino-6-chloro-9-(4-methoxy-3,5-dimethyl-pyridin-2-ylmethyl)-7-(tetrahydro-pyran-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 2-bromomethyl-tetrahydro-pyran according to the general procedure 1.3. HPLC Rt: 6.08 min. ¹H-NMR (CDCl₃) δ 7.99 (s, 1H), 5.07 (s, 2H), 4.86 (s, 2H), 3.87 (d, 2H), 3.74 (s, 3H), 2.33 (s, 3H), 2.18 (s, 3H), 1.86 (m, 1H), 1.72-1.65 (m, 2H), 1.25-1.03 (m, 6H).

### Example 51

### 2-Amino-6-chloro-9-(4-methoxy-3,5-dimethyl-pyridin-2-ylmethyl)-7-[2-(1-methyl-pyrrolidin-2-yl)-ethyl]-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 2-(2-Chloro-ethyl)-1-methyl-pyrrolidine according to the general procedure 1.3. HPLC Rt: 3.93 min. ¹H-NMR (CDCl₃) δ 8.03 (s, 1H), 5.07 (s, 2H), 4.81 (s, 2H), 4.16-4.02 (m, 2H), 3.76 (s, 3H), 3.05 (dd, 1H), 2.32 (s, 3H), 2.31 (s, 3H), 2.18 (s, 3H), 2.13-2.09 (m, 2H), 1.77-1.68 (m, 6H).

### Example 52

### 2-Amino-6-chloro-7-(2-ethoxy-ethyl)-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromo-2-ethoxy-ethane according to the general procedure 1.3. HPLC Rt: 4.93 min. ¹H-NMR (CDCl₃) δ 8.04 (s, 1H), 5.07 (s, 2H), 4.86 (s, 2H), 4.26 (t, 2H), 3.75 (s, 3H), 3.73 (t, 2H), 3.53 (q, 2H), 2.31 (s, 3H), 2.18 (s, 3H), 1.14 (t, 3H).

### Example 53

### 2-Amino-6-chloro-9-(4-methoxy-3,5-dimethyl-pyridin-2-ylmethyl)-7-(3-methyl-butyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromo-3-methyl-butane according to the general procedure 1.3. HPLC Rt: 5.83 min. ¹H-NMR (CDCl₃) δ 8.04 (s, 1H), 5.06 (s, 2H), 4.86 (s, 2H), 4.06 (t, 2H), 3.75 (s, 3H), 2.30 (s, 3H), 2.18 (s, 3H), 1.72-1.62 (m, 3H), 0.97 (d, 6H).

### Example 54

### 2-Amino-6-chloro-7-hex-5-enyl-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 6-bromo-hex-1-ene according to the general procedure 1.3. HPLC Rt: 5.68 min. ¹H-NMR (CDCl₃) δ 8.03 (s, 1H), 5.81-5.77 (m, 1H), 5.06 (s, 2H), 5.03-4.93 (m, 2H), 4.86 (s, 2H), 4.09 (t, 2H), 3.75 (s, 3H), 2.30 (s, 3H), 2.18 (s, 3H), 2.13-2.07 (m, 2H), 2.81-1.72 (m, 2H), 1.51-1.43 (m, 2H).

### Example 55

### 2-Amino-6-chloro-7-hex-5-ynyl-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 6-bromo-hex-1-yne according to the general procedure 1.3. HPLC Rt: 5.54 min. ¹H-NMR (CDCl₃) δ 8.04 (s, 1H), 5.06 (s, 2H), 4.84 (s, 2H), 4.09 (t, 2H), 3.75 (s, 3H), 2.30 (s, 3H), 2.26 (dt, 2H), 2.18 (s, 3H), 1.95 (m, 1H), 1.90-1.93 (m, 2H), 1.64-1.60 (m, 2H).

### Example 56

### 2-Amino-7-(1H-benzoimidazol-2-ylmethyl)-6-chloro-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 2-chloromethyl-1H-benzoimidazole according to the general procedure 1.3. HPLC Rt: 4.62 min. ¹H-NMR (CDCl₃) δ 10.38 (bs, 1H), 8.05 (s, 1H), 7.76 (m, 1H), 7.37 (m, 1H), 7.25 (m, 2H), 5.62 (s, 2H), 5.14 (s, 2H), 4.86 (s, 2H), 3.78 (s, 3H), 2.33 (s, 3H), 2.23 (s, 3H).

### Example 57

### 2-Amino-6-chloro-9-(4-methoxy-3,5-dimethyl-pyridin-2-ylmethyl)-7-(2-piperidin-1-yl-ethyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-(2-Chloro-ethyl)-piperidine according to the general procedure 1.3. HPLC Rt: 4.03 min. ¹H-NMR (CDCl₃) δ 8.06 (s, 1H), 5.06 (s, 2H), 4.86 (s, 2H), 4.18 (t, 2H), 3.75 (s, 3H), 2.67 (t, 2H), 2.49 (m, 4H), 2.31 (s, 3H), 2.18 (s, 3H), 1.55-1.53 (m, 4H), (m, 4H), 1.35 (m, 2H).

### Example 58

### 2-Amino-6-chloro-9-(4-methoxy-3,5-dimethyl-pyridin-2-ylmethyl)-7-(2-pyrrolidin-1-yl-ethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-(2-chloro-ethyl)-pyrrolidine according to the general procedure 1.3. HPLC Rt: 4.09 min. ¹H-NMR (CDCl₃) δ 8.05 (s, 1H), 5.06 (s, 2H), 4.89 (s, 2H), 4.22 (t, 2H), 3.75 (s, 3H), 2.86 (t, 2H), 2.64 (m, 4H), 2.30 (s, 3H), 2.19 (s, 3H), 1.79 (m, 4H).

### Example 59

### 2-Amino-6-chloro-7-(2-diisopropylamino-ethyl)-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with (2-chloro-ethyl)-diisopropyl-amine according to the general procedure 1.3. HPLC Rt: 4.10 min. ¹H-NMR (CDCl₃) δ 8.05 (s, 1H), 5.06 (s, 2H), 4.83 (s, 2H), 4.03 (dd, 2H), 3.75 (s, 3H), 3.05 (m, 2H), 2.75 (dd, 2H), 2.31 (s, 3H), 2.19 (s, 3H), 0.99 (s, 6H), 0.98 (s, 6H).

### Example 60

### 2-Amino-6-chloro-7-(2-fluoro-benzyl)-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromomethyl-2-fluoro-benzene according to the general procedure 1.3. HPLC Rt: 5.53 min. ¹H-NMR (CDCl₃) δ 8.06 (s, 1H), 7.24 (m, 1H), 7.15 (m, 1H), 7.10-7.04 (m, 2H), 5.37 (s, 2H), 5.13 (s, 2H), 4.84 (s, 2H), 3.75 (s, 3H), 2.32 (s, 3H), 2.20 (s, 3H).

### Example 61

### 2-Amino-6-chloro-9-(4-methoxy-3,5-dimethyl-pyridin-2-ylmethyl)-7-(3-nitro-benzyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-chloromethyl-3-nitro-benzene according to the general procedure 1.3. HPLC Rt: 5.27 min. ¹H-NMR (CDCl₃) δ 8.20 (d, 2H), 8.04 (s, 1H), 7.51 (d, 2H), 5.38 (s, 2H), 5.13 (s, 2H), 4.85 (s, 2H), 3.76 (s, 3H), 2.32 (s, 3H), 2.22 (s, 3H).

### Example 62

### 2-Amino-7-(2-bromo-ethyl)-6-chloro-9-(4-methoxy-3,5-dimethyl-pyridin-2-ylmethyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one according to the general procedure 1.3 using 1,2-dibromo-ethane as solvent. HPLC Rt: 4.93 min. ¹H-NMR (CDCl₃) δ 8.04 (s, 1H), 5.07 (s, 2H), 4.92 (s, 2H), 4.45 (t, 2H), 3.75 (s, 3H), 3.63 (t, 2H), 2.31 (s, 3H), 2.19 (s, 3H).

### Example 63

### 2-Amino-6-chloro-7,9-bis-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 2-chloromethyl-4-methoxy-3,5-dimethyl-pyridine according to the general procedure 1.3. HPLC Rt: 4.57 min. ¹H-NMR (CDCl₃) δ 8.11 (s, 1H), 8.04 (s, 1H), 5.28 (s, 2H), 5.12 (s, 2H), 4.89 (s, 2H), 3.75 (s, 6H), 2.31 (s, 3H), 2.27 (s, 3H), 2.19 (s, 6H).

### Example 64

### 2-Amino-6-chloro-7-cyclohexylmethyl-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with bromomethyl-cyclohexane according to the general procedure 1.3. HPLC Rt: 6.06 min. ¹H-NMR (CDCl₃) δ 8.05 (s, 1H), 5.06 (s, 2H), 4.86 (s, 2H), 4.18 (d, 2H), 3.75 (s, 3H), 2.31 (s, 3H), 2.18 (s, 3H), 2.12 (m, 1H), 1.70-1.64 (m, 5H), 1.70-1.64 (m, 5H).

### Example 65

### 2-Amino-6-chloro-7-(3-fluoro-benzyl)-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromomethyl-3-fluoro-benzene according to the general procedure 1.3. HPLC Rt: 5.73 nm in. ¹H-NMR (CDCl₃) δ 8.08 (s, 1H), 7.32-7.26 (m, 1H), 7.12 (m, 1H), 7.06 (m, 1H), 6.98-6.94 (m, 1H), 5.28 (s, 2H), 5.12 (s, 2H), 4.88 (s, 2H), 3.75 (s, 3H), 2.31 (s, 3H), 2.20 (s, 3H).

### Example 66

### 2-Amino-6-chloro-7-(4-fluoro-benzyl)-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromomethyl-4-fluoro-benzene according to the general procedure 1.3. HPLC Rt: 5.79 min. ¹H-NMR (CDCl₃) δ 8.03 (s, 1H), 7.33 (m, 2H), 7.01 (m, 2H), 5.25 (s, 2H), 5.10 (s, 2H), 4.80 (s, 2H), 3.75 (s, 3H), 2.31 (s, 3H), 2.20 (s, 3H).

### Example 67

### 2-Amino-6-chloro-9-(4-methoxy-3,5-dimethyl-pyridin-2-ylmethyl)-7-(2-methyl-benzyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-Bromomethyl-2-methyl-benzene according to the general procedure 1.3. HPLC Rt: 5.91 min. ¹H-NMR (CDCl₃) δ 8.07 (s, 1H), 7.26-7.19 (m, 3H), 6.93 (m, 1H), 5.25 (s, 2H), 5.14 (s, 2H), 4.87 (s, 2H), 3.75 (s, 3H), 2.38 (s, 3H), 2.31 (s, 3H), 2.20 (s, 3H).

### Example 68

### 2-Amino-6-chloro-9-(4-methoxy-3,5-dimethyl-pyridin-2-ylmethyl)-7-(4-methyl-benzyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-Bromomethyl-4-methyl-benzene according to the general procedure 1.3. HPLC Rt: 5.97 min. ¹H-NMR (CDCl₃) δ 8.05 (s, 1H), 7.23 (d, 2H), 7.12 (d, 2H), 5.24 (s, 2H), 5.10 (s, 2H), 4.87 (s, 2H), 3.75 (s, 3H), 2.32 (s, 3H), 2.30 (s, 3H), 2.20 (s, 3H).

### Example 69

### Acetic acid 4-[2-amino-6-chloro-9-(4-methoxy-3,5-dimethyl-pyridin-2-ylmethyl)-8-oxo-8,9-dihydro-purin-7-yl]-butyl ester

The title compound was obtained by alkylation of 2-amino-6-chloro-9-(4-methoxy-3,5-dimethyl-pyridin-2-ylmethyl)-7,9-dihydro-purin-8-one with acetic acid 4-iodo-butyl ester according to the general procedure 1.3. HPLC Rt: 4.97 min. ¹H-NMR (CDCl₃) δ 8.02 (s, 1H), 5.06 (s, 2H), 4.89 (s, 2H), 4.09 (m, 4H), 3.75 (s, 3H), 2.30 (s, 3H), 2.18 (s, 3H), 2.04 (s, 3H), 1.87-1.70 (m, 4H).

### Example 70

### [2-Amino-6-chloro-9-(4-methoxy-3,5-dimethyl-pyridin-2-ylmethyl)-8-oxo-8,9-dihydro-purin-7-yl]-acetonitrile

The title compound was obtained by alkylation of 2-amino-6-chloro-9-(4-methoxy-3,5-dimethyl-pyridin-2-ylmethyl)-7,9-dihydro-purin-8-one with bromoacetonitrile according to the general procedure 1.3. HPLC R.t. 4.35 min. ¹H-NMR (CDCl₃) δ 8.04 (s, 1H), 5.17 (s, 2H), 4.99 (s, 2H), 4.90 (br s, 2H), 3.76 (s, 3H), 2.31 (s, 3H), 2.19 (s, 3H).

### Example 71

### 2-Amino-6-chloro-9-(4-methoxy-3,5-dimethyl-pyridin-2-ylmethyl)-7-(3,4,4-trifluoro-but-3-enyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 4-bromo-1,1,2-trifluoro-but-1-ene according to the general procedure 1.3. HPLC Rt: 5.33 min. ¹H-NMR (CDCl₃) δ 8.03 (s, 1H), 5.06 (s, 2H), 4.90 (s, 2H), 4.30 (t, 2H), 3.75 (s, 3H), 2.82-2.73 (m, 2H), 2.31 (s, 3H), 2.19 (s, 3H).

### Example 72

### 2-Amino-6-chloro-7-(3-methoxy-benzyl)-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromomethyl-3-methoxy-benzene according to the general procedure 1.3. HPLC Rt: 5.47 min. ¹H-NMR (CDCl₃) δ 8.07 (s, 1H), 7.23 (t, 1H), 6.91-6.88 (m, 2H), 6.80-6.78 (m, 1H), 5.26 (s, 2H), 5.10 (s, 2H), 4.89 (s, 2H), 3.79 (s, 3H), 3.74 (s, 3H), 2.31 (s, 3H), 2.19 (s, 3H).

### Example 73

### 2-Amino-6-chloro-7-(4-methoxy-benzyl)-9-(4-methoxy-3,5-dimethyl-pyridin-2-ylmethyl)-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 1-Bromomethyl-4-methoxy-benzene according to the general procedure 1.3. HPLC Rt: 5.45 min. ¹H-NMR (CDCl₃) δ 8.07 (s, 1H), 7.30 (d, 2H), 6.85 (d, 2H), 5.21 (s, 2H), 5.09 (s, 2H), 4.85 (s, 2H), 3.79 (s, 3H), 3.75 (s, 3H), 2.31 (s, 3H), 2.19 (s, 3H).

### Example 74

### 2-Amino-6-chloro-9-(4-methoxy-3,5-dimethyl-pyridin-2-ylmethyl)-7-(2-oxo-oxazolidin-5-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 5-chloromethyl-oxazolidin-2-one according to the general procedure 1.3. HPLC Rt: 3.97 min. ¹H-NMR (CDCl₃) δ 8.01 (s, 1H), 5.91 (s, 1H), 5.06 (s, 2H), 5.03 (m, 1H), 5.02 (s, 2H), 4.47-4.41 (, 1H), 4.35-4.31 (m, 1H), 3.75 (s, 3H), 3.70 (t, 1H), 3.55 (m, 1H), 2.30 (s, 3H), 2.18 (s, 3H).

### Example 75

### {3-[2-Amino-6-chloro-9-(4-methoxy-3,5-dimethyl-pyridin-2-ylmethyl)-8-oxo-8,9-dihydro-purin-7-yl]-propyl}-phosphonic acid diethyl ester

The title compound was obtained by alkylation of 2-amino-6-chloro-9-(4-methoxy-3,5-dimethyl-pyridin-2-ylmethyl)-7,9-dihydro-purin-8-one with (3-bromo-propyl)-phosphonic acid diethyl ester according to the general procedure 1.3. HPLC Rt: 4.85 min. ¹H-NMR (CDCl₃) δ 8.05 (s, 1H), 5.05 (s, 2H), 4.89 (s, 2H), 4.15 (m, 6H), 3.74 (s, 3H), 2.30 (s, 3H), 2.18 (s, 3H), 2.08 (m, 2H), 1.88-1.79 (m, 2H), 2.314 (t, 6H).

### Example 76

### 2-Amino-6-chloro-9-(4-methoxy-3,5-dimethyl-pyridin-2-ylmethyl)-7-(3-methyl-benzyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromomethyl-3-methyl-benzene according to the general procedure 1.3. HPLC Rt: 5.74 min. ¹H-NMR (CDCl₃) δ 8.06 (s, 1H), 7.20 (m, 1H), 7.13-7.06 (m, 3H), 5.24 (s, 2H), 5.11 (s, 2H), 4.87 (s, 2H), 3.75 (s, 3H), 2.32 (s, 3H), 2.31 (s, 3H), 2.19 (s, 3H).

### Example 77

### 2-Amino-6-chloro-7-(3-isopropylamino-propyl)-9-(4-methoxy-3,5-dimethyl-pyridin-2-ylmethyl)-7,9-dihydro-purin-8-one

The title compound was obtained by alkylation of isopropylamine with 2-amino-7-(3-bromo-propyl)-6-chloro-9-(4-methoxy-3,5-dimethyl-pyridin-2-ylmethyl)-7,9-dihydro-purin-8-one according to the general procedure 1.4. HPLC Rt: 4.16 min. ¹H-NMR (CDCl₃) δ 8.02 (s, 1H), 5.06 (s, 2H), 4.90 (s, 2H), 4.15 (t, 2H), 3.75 (s, 3H), 2.79 (m, 1H), 2.67 (t, 2H), 2.31 (s, 3H), 2.19 (s, 3H), 2.03 (m, 2H), 1.05 (d, 3H), 1.03 (d, 3H).

### Example 78

### 2-Amino-7-(3-sec-butylamino-propyl)-6-chloro-9-(4-methoxy-3,5-dimethyl-pyridin-2-ylmethyl)-7,9-dihydro-purin-8-one

The title compound was obtained by alkylation of sec-Butylamine with 2-Amino-7-(3-bromo-propyl)-6-chloro-9-(4-methoxy-3,5-dimethyl-pyridin-2-ylmethyl)-7,9-dihydro-purin-8-one according to the general procedure 1.4. HPLC Rt: 4.13 min. ¹H-NMR (CDCl₃) δ 8.02 (s, 1H), 5.30 (s, 2H), 5.06 (s, 2H), 4.15 (t, 2H), 3.75 (s, 3H), 2.74-2.65 (m, 2H), 2.53 (m, 2H), 2.31 (s, 3H), 2.19 (s, 3H), 2.03 (m, 2H), 1.53-1.47 (m, 1H), 1.33-1.24 (m, 1H), 1.01 (d, 3H), 1.03 (t, 3H).

### Example 79

### 2-Amino-6-chloro-7-(3,4-difluoro-benzyl)-9-(4-methoxy-3,5-dimethyl-pyridin-2-ylmethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 4-bromomethyl-1,2-difluoro-benzene according to the general procedure 1.3. HPLC Rt: 5.66 min. ¹H-NMR (CDCl₃) δ 8.06 (s, 1H), 8.04 (m, 1H), 7.26-7.08 (m, 2H), 5.24 (s, 2H), 5.11 (s, 2H), 4.85 (s, 2H), 3.75 (s, 3H), 2.32 (s, 3H), 2.20 (s, 3H).

### Example 80

### 4-[2-Amino-6-chloro-9-(4-methoxy-3,5-dimethyl-pyridin-2-ylmethyl)-8-oxo-8,9-dihydro-purin-7-ylmethyl]-benzoic acid methyl ester

The title compound was obtained by alkylation of 2-amino-6-chloro-9-(4-methoxy-3,5-dimethyl-pyridin-2-ylmethyl)-7,9-dihydro-purin-8-one with 4-bromomethyl-benzoic acid methyl ester according to the general procedure 1.3. HPLC Rt: 5.43 min. ¹H-NMR (CDCl₃) δ 8.05 (s, 1H), 8.01 (d, 2H), 7.39 (d, 2H), 5.38 (s, 2H), 5.13 (s, 2H), 4.81 (s, 2H), 3.91 (s, 3H), 3.76 (s, 3H), 2.32 (s, 3H), 2.21 (s, 3H).

### Example 81

### 2-Amino-6-chloro-9-(4-methoxy-3,5-dimethyl-pyridin-2-ylmethyl)-7-pent-2-enyl-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-bromo-pent-2-ene according to the general procedure 1.3. HPLC Rt: 5.53 min. ¹H-NMR (CDCl₃) δ 8.04 (s, 1H), 5.70 (m, 1H), 5.57 (m, 1H), 5.07 (s, 2H), 4.88 (s, 2H), 4.62 (m, 2H), 3.75 (s, 3H), 2.31 (s, 3H), 2.19 (s, 3H), 2.05 (m, 2H), 0.96 (t, 3H).

### Example 82

### 2-Amino-6-chloro-9-(4-methoxy-3,5-dimethyl-pyridin-2-ylmethyl)-7-pyridin-2-ylmethyl-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 2-Chloromethyl-pyridine according to the general procedure 1.3. HPLC Rt: 4.34 min. ¹H-NMR (CDCl₃) δ 8.55 (m, 1H), 8.06 (s, 1H), 8.04 (td, 1H), 7.17 (m, 2H), 5.42 (s, 2H), 5.13 (s, 2H), 4.85 (s, 2H), 3.75 (s, 3H), 2.31 (s, 3H), 2.19 (s, 3H).

### Example 83

### 2-Amino-6-chloro-9-(4-methoxy-3,5-dimethyl-pyridin-2-ylmethyl)-7-pyridin-3-ylmethyl-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 3-Chloromethyl-pyridine according to the general procedure 1.3. HPLC Rt: 4.01 min. ¹H-NMR (CDCl₃) δ 8.65 (d, 1H), 8.54 (dd, 1H), 8.04 (s, 1H), 7.68 (m, 1H), 7.26 (m, 1H), 5.30 (s, 2H), 5.10 (s, 2H), 4.87 (s, 2H), 3.75 (s, 3H), 2.30 (s, 3H), 2.19 (s, 3H).

### Example 84

### 2-Amino-6-chloro-9-(4-methoxy-3,5-dimethyl-pyridin-2-ylmethyl)-7-pyridin-4-ylmethyl-7,9-dihydro-purin-8-one

The title compound was obtained by alkylation of 2-Amino-6-chloro-9-(4-methoxy-3,5-dimethyl-pyridin-2-ylmethyl)-7,9-dihydro-purin-8-one with 4-bromomethyl-pyridine according to the general procedure 1.3. HPLC Rt: 4.60 min. ¹H-NMR (CDCl₃) δ 8.58 (d, 2H), 8.06 (s, 1H), 7.25 (d, 2H), 5.30 (s, 2H), 5.14 (s, 2H), 4.84 (s, 2H), 3.76 (s, 3H), 2.33 (s, 3H), 2.22 (s, 3H).

### Example 85

### 4-[2-Amino-6-chloro-9-(4-methoxy-3,5-dimethyl-pyridin-2-ylmethyl)-8-oxo-8,9-dihydro-purin-7-yl]-butyric acid methyl ester

The title compound was obtained by alkylation of 2-amino-6-chloro-9-(4-methoxy-3,5-dimethyl-pyridin-2-ylmethyl)-7,9-dihydro-purin-8-one with 4-bromo-butyric acid methyl ester according to the general procedure 1.3. HPLC Rt: 4.96 min. ¹H-NMR (CDCl₃) δ 8.04 (s, 1H), 5.06 (s, 2H), 4.86 (s, 2H), 4.14 (t, 2H), 3.75 (s, 3H), 3.66 (s, 3H), 2.44 (t, 2H), 2.31 (s, 3H), 2.19 (s, 3H), 2.12 (m, 2H).

### Example 86

### 3-{2-[2-Amino-6-chloro-9-(4-methoxy-3,5-dimethyl-pyridin-2-ylmethyl)-8-oxo-8,9-dihydro-purin-7-yl]-ethyl}-1H-quinazoline-2,4-dione

The title compound was obtained by alkylation of 2-amino-6-chloro-9-(4-methoxy-3,5-dimethyl-pyridin-2-ylmethyl)-7,9-dihydro-purin-8-one with 3-(2-chloro-ethyl)-1H-quinazoline-2,4-dione according to the general procedure 1.3. HPLC Rt: 4.73 min. ¹H-NMR (CDCl₃) δ 9.10 (bs, 1H), 8.01 (d, 1H), 7.99 (s, 1H), 7.52 (m, 1H), 7.17 (m, 1H), 6.89 (d, 1H), 4.97 (s, 2H), 4.84 (bs, 2H), 4.47 (s, 4H), 3.69 (s, 3H), 2.19 (s, 3H), 2.14 (s, 3H).

### Example 87

### 2-Amino-6-chloro-7-(2-isopropylamino-ethyl)-9-(4-methoxy-3,5-dimethyl-pyridin-2-ylmethyl)-7,9-dihydro-purin-8-one

The title compound was obtained by alkylation of isopropylamine with 2-Amino-7-(2-bromo-ethyl)-6-chloro-9-(4-methoxy-3,5-dimethyl-pyridin-2-ylmethyl)-7,9-dihydro-purin-8-one according to the general procedure 1.4. HPLC Rt: 3.87 min. ¹H-NMR (CDCl₃) δ 8.01 (s, 1H), 5.08 (s, 2H), 4.85 (s, 2H), 4.21 (t, 2H), 3.75 (s, 3H), 3.08 (t, 2H), 2.95 (m, 1H), 2.31 (s, 3H), 2.18 (s, 3H), 1.08 (d, 6H).

### Example 88

### 2-Amino-7-(2-amino-ethyl)-6-chloro-9-(4-methoxy-3,5-dimethyl-pyridin-2-ylmethyl)-7,9-dihydro-purin-8-one

The title compound was obtained by alkylation of ammonia with 2-Amino-7-(3-bromo-propyl)-6-chloro-9-(4-methoxy-3,5-dimethyl-pyridin-2-ylmethyl)-7,9-dihydro-purin-8-one according to the general procedure 1.4. HPLC Rt: 3.71 min. ¹H-NMR (CDCl₃) δ 8.02 (s, 1H), 5.06 (s, 2H), 4.81 (s, 2H), 4.15 (t, 2H), 3.75 (s, 3H), 3.08 (t, 2H), 2.31 (s, 3H), 2.19 (s, 3H).

### Example 89

### 2-Amino-6-chloro-9-(4-methoxy-3,5-dimethyl-pyridin-2-ylmethyl)-7-(2-pyrrol-1-yl-ethyl)-7,9-dihydro-purin-8-one

The title compound was obtained by alkylation of 2-amino-6-chloro-9-(4-methoxy-3,5-dimethyl-pyridin-2-ylmethyl)-7,9-dihydro-purin-8-one with 1-(2-chloro-ethyl)-1H-pyrrole according to the general procedure 1.3. HPLC Rt: 4.96 min. ¹H-NMR (CDCl₃) δ 8.05 (s, 1H), 6.66 (t, 2H), 6.11 (t, 2H), 5.06 (s, 2H), 4.83 (s, 2H), 4.39 (dd, 2H), 4.24 (dd, 2H), 3.75 (s, 3H), 2.31 (s, 3H), 2.19 (s, 3H).

### Example 90

### 7-Acetyl-2-amino-6-chloro-9-(4-methoxy-3,5-dimethyl-pyridin-2-ylmethyl)-7,9-dihydro-purin-8-one

The title compound was obtained by acylation of 2-amino-6-chloro-9-(4-methoxy-3,5-dimethyl-pyridin-2-ylmethyl)-7,9-dihydro-purin-8-one with acetic anhydride according to the general procedure 1.5. HPLC Rt: 4.58 min. ¹H-NMR (CDCl₃) δ 8.05 (s, 1H), 5.03 (s, 2H), 4.98 (s, 2H), 3.75 (s, 3H), 2.74 (s, 3H), 2.31 (s, 3H), 2.19 (s, 3H).

### Example 91

### 2-Amino-6-chloro-9-(4-methoxy-3,5-dimethyl-pyridin-2-ylmethyl)-7-(2-[1,2,4]triazol-1-yl-ethyl)-7,9-dihydro-purin-8-one

The title compound was obtained by alkylation of 1H-[1,2,4]triazole with 2-Amino-7-(2-bromo-ethyl)-6-chloro-9-(4-methoxy-3,5-dimethyl-pyridin-2-ylmethyl)-7,9-dihydro-purin-8-one according to the general procedure 1.4. HPLC Rt: 3.99 min. ¹H-NMR (CDCl₃) δ 8.13 (s, 1H), 8.07 (s, 1H), 7.92 (s, 1H), 5.06 (s, 2H), 4.83 (s, 2H), 4.62 (t, 2H), 4.54 (t, 2H), 3.76 (s, 3H), 2.31 (s, 3H), 2.19 (s, 3H).

### Example 92

### 2-Amino-6-chloro-9-(4-methoxy-3,5-dimethyl-pyridin-2-ylmethyl)-7-[2-(4-methyl-4H-[1,2,4]triazol-3-ylsulfanyl)-ethyl]-7,9-dihydro-purin-8-one

The title compound was obtained by alkylation of 4-Methyl-4H-[1,2,4]triazole-3-thiol with 2-Amino-7-(2-bromo-ethyl)-6-chloro-9-(4-methoxy-3,5-dimethyl-pyridin-2-ylmethyl)-7,9-dihydro-purin-8-one according to the general procedure 1.4. HPLC Rt: 4.79 min. ¹H-NMR (CDCl3) δ 8.03 (s, 1H), 5.06 (s, 2H), 4.83 (s, 2H), 4.51 (t, 2H), 3.76 (s, 3H), 3.70 (t, 2H), 2.71 (s, 3H), 2.30 (s, 3H), 2.19 (s, 3H).

### Example 93

### 2-Amino-6-chloro-7-(2-imidazol-1-yl-ethyl)-9-(4-methoxy-3,5-dimethyl-pyridin-2-ylmethyl)-7,9-dihydro-purin-8-one

The title compound was obtained by alkylation of 1H-imidazole with 2-Amino-7-(2-bromo-ethyl)-6-chloro-9-(4-methoxy-3,5-dimethyl-pyridin-2-ylmethyl)-7,9-dihydro-purin-8-one according to the general procedure 1.4. HPLC Rt: 3.72 min. ¹H-NMR (CDCl₃) δ 8.05 (s, 1H), 7.49 (s, 1H), 7.03 (m, 1H), 6.98 (m, 1H), 5.06 (s, 2H), 4.82 (s, 2H), 4.54 (m, 2H), 4.43 (m, 2H), 3.76 (s, 3H), 2.31 (s, 3H), 2.19 (s, 3H).

### Example 94

### 2-Amino-6-chloro-9-(4-methoxy-3,5-dimethyl-pyridin-2-ylmethyl)-7-[2-(4-methyl-piperazin-1-yl)-ethyl]-7,9-dihydro-purin-8-one

The title compound was obtained by alkylation of 1-Methyl-piperazine with 2-Amino-7-(2-bromo-ethyl)-6-chloro-9-(4-methoxy-3,5-dimethyl-pyridin-2-ylmethyl)-7,9-dihydro-purin-8-one according to the general procedure 1.4. HPLC Rt: 3.81 min. ¹H-NMR (CDCl₃) δ 8.05 (s, 1H), 5.06 (s, 2H), 4.83 (s, 2H), 4.17 (t, 2H), 3.76 (s, 3H), 2.71 (t, 2H), 2.67-2.55 (m, 4H), 2.45-2.35 (m, 4H), 2.31 (s, 3H), 2.25 (s, 3H), 2.19 (s, 3H).

### Example 95

### 2-Amino-6-chloro-9-(4-methoxy-3,5-dimethyl-pyridin-2-ylmethyl)-7-pentanoyl-7,9-dihydro-purin-8-one

The title compound was obtained by acylation of 2-Amino-6-chloro-9-(4-methoxy-3,5-dimethyl-pyridin-2-ylmethyl)-7,9-dihydro-purin-8-one with pentanoyl chloride according to the general procedure 1.5. HPLC Rt: 6.04-min. ¹H-NMR (CDCl₃) δ 8.05 (s, 1H), 5.17 (s, 2H), 5.03 (s, 2H), 3.76 (s, 3H), 3.15 (t, 2H), 2.31 (s, 3H), 2.20 (s, 3H), 1.42 (m, 2H), 1.78 (m, 2H), 0.95 (t, 3H).

### Example 96

### 7-(2-(1H-pyrrol-1-yl)ethyl)-2-amino-6-chloro-9-((4-iodo-3,5-dimethylpyridin-2-yl)methyl)-7H-purin-8(9H)-one

The title compound was obtained by alkylation of 2-Amino-6-chloro-9-(4-iodo-3,5-dimethyl-pyridin-2-ylmethyl)-7,9-dihydro-purin-8-one with 1-(2-chloroethyl)-1H-pyrrole according to the general procedure 1.3. HPLC Rt: 6.40 min. ¹H-NMR (CDCl₃) δ 7.96 (s, 1H), 6.16 (s, 2H), 6.11 (s, 2H), 5.13 (s, 2H), 4.81 (s, 2H), 4.39 (m, 2H), 4.24 (m, 2H), 2.55 (s, 3H), 2.36 (s, 3H).

### Example 97

### 2-amino-6-chloro-9-((4-iodo-3,5-dimethylpyridin-2-yl)methyl)-7-(4-methylpent-3-enyl)-7H-purin-8(9H)-one

The title compound was obtained by alkylation of 2-Amino-6-chloro-9-(4-methoxy-3,5-dimethyl-pyridin-2-ylmethyl)-7,9-dihydro-purin-8-one with 5-bromo-2-methylpent-2-ene according to the general procedure 1.3. HPLC Rt: 6.86 nm in. ¹H-NMR (CDCl₃) δ 7.96 (s, 1H), 5.16 (m, 1H), 5.13 (s, 2H), 4.81 (s, 2H), 4.03 (m, 2H), 2.55 (s, 3H), 2.45 (m, 2H), 2.35 (s, 3H), 1.67 (s, 3H), 1.58 (s, 3H).

### Example 98

### 2-amino-6-chloro-7-(2-(diisopropylamino)ethyl)-9-((4-iodo-3,5-dimethylpyridin-2-yl)methyl)-7H-purin-8(9H)-one

The title compound was obtained by alkylation of 2-Amino-6-chloro-9-(4-iodo-3,5-dimethyl-pyridin-2-ylmethyl)-7,9-dihydro-purin-8-one with 2-diisopropylamino-ethyl bromide according to the general procedure 1.3. HPLC Rt: 5.21 nm in. ¹H-NMR (CDCl₃) δ 7.96 (s, 1H), 5.13 (s, 2H), 4.78 (s, 2H), 4.03 (m, 2H), 3.03 (m, 2H), 2.74 (m, 2H), 2.55 (s, 3H), 2.36 (s, 3H), 0.98 (d, 12H).

### Example 99

### 2-amino-6-chloro-9-((4-iodo-3,5-dimethylpyridin-2-yl)methyl)-7-(pyridin-2-ylmethyl)-7H-purin-8(9H)-one

The title compound was obtained by alkylation of 2-Amino-6-chloro-9-(4-iodo-3,5-dimethyl-pyridin-2-ylmethyl)-7,9-dihydro-purin-8-one with 2-(bromomethyl)pyridine according to the general procedure 1.3. HPLC Rt: 6.05 min. ¹H-NMR (CDCl₃) δ 8.55 (m, 1H), 8.11 (s, 1H), 7.65 (td, 1H), 7.18 (m, 2H), 5.42 (s, 2H), 5.19 (s, 2H), 4.80 (s, 2H), 2.47 (s, 3H), 2.30 (s, 3H).

### Example 100

### 2-amino-7-(2-(5-amino-1H-tetrazol-1-yl)ethyl)-6-chloro-9-((4-methoxy-3,5-dimethylpyridin-2-yl)methyl)-7H-purin-8(9H)-one

The title compound was obtained by alkylation of 5-amino-tetrazole with 2-Amino-7-(2-bromo-ethyl)-6-chloro-9-(4-methoxy-3,5-dimethyl-pyridin-2-ylmethyl)-7,9-dihydro-purin-8-one according to the general procedure 1.4. HPLC Rt: 4.06 min. ¹H-NMR (CDCl₃) δ 8.07 (s, 1H), 5.03 (s, 2H), 4.86 (s, 2H), 4.81 (t, 2H), 4.57 (t, 2H), 4.34 (s, 2H), 3.76 (s, 3H), 2.30 (s, 3H), 2.20 (s, 3H).

### Example 101

### 3-((2-amino-6-chloro-9-((4-methoxy-3,5-dimethylpyridin-2-yl)methyl)-8-oxo-8,9-dihydro-7H-purin-7-yl)methyl)benzo[d]thiazol-2(3H)-one

The title compound was obtained by alkylation of 2-Amino-6-chloro-9-(4-methoxy-3,5-dimethyl-pyridin-2-ylmethyl)-7,9-dihydro-purin-8-one with 2-diisopropylamino-ethyl bromide according to the general procedure 1.3. HPLC Rt: 5.50 min. ¹H-NMR (CDCl₃) δ 7.99 (s, 1H), 7.49 (dd, 1H), 7.45 (dd, 1H), 7.30 (m, 2H), 6.68 (s, 2H), 5.07 (s, 2H), 4.83 (s, 2H), 3.75 (s, 3H), 2.28 (s, 3H), 2.20 (s, 3H).

### Example 102

### 2-Amino-6-chloro-9-(4-iodo-3,5-dimethyl-pyridin-2-ylmethyl)-7-[2-(4-methyl-piperazin-1-yl)-ethyl]-7,9-dihydro-purin-8-one

The title compound was obtained by alkylation of 1-Methyl-piperazine with 2-Amino-6-chloro-9-(4-iodo-3,5-dimethyl-pyridin-2-ylmethyl)-7,9-dihydro-purin-8-one according to the general procedure 1.4. HPLC Rt: 4.61 min. ¹H-NMR (CDCl₃) δ 7.96 (s, 1H), 5.13 (s, 2H), 4.79 (s, 2H), 4.18 (t, 2H), 2.73 (t, 2H), 2.69-2.50 (m, 4H), 2.55 (s, 3H), 2.50-2.30 (m, 4H), 2.35 (s, 3H), 2.25 (s, 3H).

### Example 103

### 2-amino-7-(2-bromoethyl)-6-chloro-9-((4-iodo-3,5-dimethylpyridin-2-yl)methyl)-7H-purin-8(9H)-one

The title compound was obtained by alkylation of 2-Amino-6-chloro-9-(4-iodo-3,5-dimethyl-pyridin-2-ylmethyl)-7,9-dihydro-purin-8-one according to the general procedure 1.3 using 1,2-dibromo-ethane as solvent. HPLC Rt: 6.02 nm. ¹H-NMR (DMSO) δ 7.98 (s, 1H), 6.69 (s, 2H), 5.10 (s, 2H), 4.33 (t, 2H), 3.73 (t, 2H), 2.50 (s, 3H), 2.31 (s, 3H).

### Example 104

### Ethyl 4-(2-(2-amino-6-chloro-9-((4-iodo-3,5-dimethylpyridin-2-yl)methyl)-8-oxo-8,9-dihydro-7H-purin-7-yl)ethyl)piperazine-1-carboxylate

The title compound was obtained by alkylation of ethyl piperazine-1-carboxylate with 2-Amino-7-(2-bromo-ethyl)-6-chloro-9-(4-iodo-3,5-dimethyl-pyridin-2-ylmethyl)-7,9-dihydro-purin-8-one according to the general procedure 1.4. HPLC Rt: 5.88 min. ¹H-NMR (CDCl₃) δ 7.93 (s, 1H), 5.12 (s, 2H), 4.81 (s, 2H), 4.42 (m, 2H), 4.36 (m, 2H), 4.13 (q, 2H), 3.41 (m, 4H), 3.29 (m, 4H), 2.56 (s, 3H), 2.36 (s, 3H), 1.25 (t, 3H).

### Example 105

### 7-acetyl-2-amino-6-chloro-9-((4-iodo-3,5-dimethylpyridin-2-yl)methyl)-7H-purin-8(9H)-one

The title compound was obtained by acylation of 2-amino-6-chloro-9-((4-iodo-3,5-dimethylpyridin-2-yl)methyl)-7H-purin-8(9H)-one with acetic anhydride according to the general procedure 1.5. HPLC Rt: 6.03 min. ¹H-NMR (CDCl₃) δ 8.12 (s, 1H), 5.36 (s, 2H), 4.66 (s, 2H), 2.48 (s, 3H), 2.39 (s, 3H), 2.07 (s, 3H).

### Example 106

### 2-Amino-6-chloro-7-(2-diisobutylamino-ethyl)-9-(4-iodo-3,5-dimethyl-pyridin-2-ylmethyl)-7,9-dihydro-purin-8-one

The title compound was obtained by alkylation of Diisobutyl-amine with 2-Amino-7-(2-bromo-ethyl)-6-chloro-9-(4-iodo-3,5-dimethyl-pyridin-2-ylmethyl)-7,9-dihydro-purin-8-one according to the general procedure 1.4. HPLC Rt: 7.24 min. ¹H-NMR (CDCl₃) δ 7.96 (s, 1H), 5.13 (s, 2H), 4.79 (s, 2H), 4.36 (s, 4H), 3.05 (d, 2H), 2.92 (d, 2H), 2.53 (s, 3H), 2.35 (s, 3H), 1.98-1.88 (m, 1H), 1.80-1.75 (m, 1H), 0.85 (d, 6H), 0.74 (d, 6H).

### Example 107

### 2-amino-6-chloro-7-(2-(dipropylamino)ethyl)-9-((4-iodo-3,5-dimethylpyridin-2-yl)methyl)-7H-purin-8(9H)-one

The title compound was obtained by alkylation of dipropylamine with 2-Amino-7-(2-bromo-ethyl)-6-chloro-9-(4-iodo-3,5-dimethyl-pyridin-2-ylmethyl)-7,9-dihydro-purin-8-one according to the general procedure 1.4. HPLC Rt: 5.56 min. ¹H-NMR (CDCl₃) δ 7.96 (s, 1H), 5.13 (s, 2H), 4.80 (s, 2H), 4.10 (m, 2H), 2.76 (t, 2H), 2.55 (s, 3H), 2.43 (m, 4H), 2.35 (s, 3H), 1.44-1.38 (m, 4H), 0.83 (t, 6H).

### Example 108

### 2-amino-6-chloro-9-((4-iodo-3,5-dimethylpyridin-2-yl)methyl)-7-(2-(isopropyl(methyl)amino)ethyl)-7H-purin-8(9H)-one

The title compound was obtained by alkylation of N-methylpropan-2-amine with 2-Amino-7-(2-bromo-ethyl)-6-chloro-9-(4-iodo-3,5-dimethyl-pyridin-2-ylmethyl)-7,9-dihydro-purin-8-one according to the general procedure 1.4. HPLC Rt: 5.03 min. ¹H-NMR (CDCl₃) δ 7.98 (s, 1H), 5.15 (s, 2H), 4.83 (s, 2H), 4.15 (t, 2H), 2.84 (m, 1H), 2.71 (t, 2H), 2.57 (s, 3H), 2.37 (s, 3H), 2.34 (s, 3H), 1.25 (d, 6H).

### Example 109

### 2-((2-amino-6-chloro-7-(2-(diisopropylamino)ethyl)-8-oxo-7H-purin-9(8H)-yl)methyl)-4-iodo-3,5-dimethylpyridine 1-oxide

The title compound was obtained by oxidation of 2-amino-6-chloro-7-(2-(diisopropylamino)ethyl)-9-((4-iodo-3,5-dimethylpyridin-2-yl)methyl)-7H-purin-8(9H)-one with 3-chloroperoxybenzoic acid according to the procedure 1.6. HPLC Rt: 4.76 min. ¹H-NMR (CDCl₃) δ 7.96 (s, 1H), 5.13 (s, 2H), 5.08 (s, 2H), 4.55 (m, 2H), 3.59 (m, 2H), 3.35 (m, 2H), 2.75 (s, 3H), 2.37 (s, 3H), 1.43 (d, 6H), 1.39 (d, 6H).

### Example 110

### 2-amino-9-((4-bromo-3,5-dimethylpyridin-2-yl)methyl)-6-chloro-7-(2-(diisopropylamino)ethyl)-7H-purin-8(9H)-one

The title compound was obtained by alkylation of diisopropyl amine with 2-Amino-7-(2-bromo-ethyl)-6-chloro-9-(4-bromo-3,5-dimethyl-pyridin-2-ylmethyl)-7,9-dihydro-purin-8-one according to the general procedure 1.4. HPLC Rt: 5.20 min. ¹H-NMR (CDCl₃) δ 8.01 (s, 1H), 5.13 (s, 2H), 4.83 (s, 2H), 4.04 (t, 2H), 3.04 (m, 2H), 2.75 (t, 2H), 2.50 (s, 3H), 2.32 (s, 3H), 0.99 (d, 12H).

### Example 111

### 2-amino-9-((4-bromo-3,5-dimethylpyridin-2-yl)methyl)-6-chloro-7-(4-methylpent-3-enyl)-7H-purin-8(9H)-one

The title compound was obtained by alkylation of 2-Amino-6-chloro-9-(4-bromo-3,5-dimethyl-pyridin-2-ylmethyl)-7,9-dihydro-purin-8-one with 1-Chloro-4-methyl-pent-3-ene according to the general procedure 1.3. HPLC Rt: 6.22 min. ¹H-NMR (CDCl₃) δ 8.04 (s, 1H), 5.16 (m, 1H), 5.13 (s, 2H), 4.81 (s, 2H), 4.04 (m, 2H), 2.50 (s, 3H), 2.44 (m, 2H), 2.32 (s, 3H), 1.67 (s, 3H), 1.59 (s, 3H).

### Example 112

### 7-(2-(1H-pyrrol-1-yl)ethyl)-2-amino-9-((4-bromo-3,5-dimethylpyridin-2-yl)methyl)-6-chloro-7H-purin-8(9H)-one

The title compound was obtained by alkylation of 2-Amino-6-chloro-9-(4-bromo-3,5-dimethyl-pyridin-2-ylmethyl)-7,9-dihydro-purin-8-one with 1-(2-Chloro-ethyl)-1H-pyrrole according to the general procedure 1.3. HPLC Rt: 6.85 min. ¹H-NMR (CDCl₃) δ 8.04 (s, 1H), 6.66 (d, 1H), 6.65 (d, 1H), 6.115 (d, 1H), 6.11 (d, 1H), 5.12 (s, 2H), 4.86 (s, 2H), 4.39 (t, 2H), 4.24 (t, 2H), 2.50 (s, 3H), 2.32 (s, 3H).

### Example 113

### 2-amino-9-(2-bromo-3,4,5-trimethoxybenzyl)-6-chloro-7-(2-(diisopropylamino)ethyl)-7H-purin-8(9H)-one

The title compound was obtained by alkylation of diisopropyl amine with 2-amino-9-(2-bromo-3,4,5-trimethoxybenzyl)-7-(2-bromoethyl)-6-chloro-7H-purin-8(9H)-one according to the general procedure 1.4. HPLC Rt: 5.50 min. ¹H-NMR (CDCl₃) δ 6.50 (s, 1H), 5.09 (s, 2H), 4.92 (s, 2H), 4.05 (t, 2H), 3.90 (s, 3H), 3.85 (s, 3H), 3.73 (s, 3H), 3.04 (m, 2H), 2.74 (t, 2H), 0.96 (d, 12H).

### Example 114

### 2-amino-9-(2-bromo-3,4,5-trimethoxybenzyl)-6-chloro-7-(4-methylpent-3-enyl)-7H-purin-8(9H)-one

The title compound was obtained by alkylation of 2-amino-9-(2-bromo-3,4,5-trimethoxybenzyl)-6-chloro-7H-purin-8(9H)-one with 1-(2-Chloro-4-methyl-pent-3-ene according to the general procedure 1.3. HPLC Rt: 7.38 min. ¹H-NMR (CDCl₃) δ 6.51 (s, 1H), 5.15 (m, 1H), 5.10 (s, 2H), 4.90 (s, 2H), 4.04 (m, 2H), 3.90 (s, 3H), 3.86 (s, 3H), 3.73 (s, 3H), 2.43 (m, 2H), 1.66 (s, 3H), 1.57 (s, 3H).

### Example 115

### 7-(2-(1H-pyrrol-1-yl)ethyl)-2-amino-9-(2-bromo-3,4,5-trimethoxybenzyl)-6-chloro-7H-purin-8(9H)-one

The title compound was obtained by alkylation of 2-amino-9-(2-bromo-3,4,5-trimethoxybenzyl)-6-chloro-7H-purin-8(9H)-one with 1-(2-Chloro-ethyl)-1H-pyrrole according to the general procedure 1.3. HPLC Rt: 6.89 min. ¹H-NMR (CDCl₃) δ 6.60 (d, 1H), 6.595 (d, 1H), 6.53 (s, 3H), 6.11 (d, 1H), 6.10 (d, 1H), 5.07 (s, 2H), 4.92 (s, 2H), 4.40 (t, 2H), 4.25 (t, 2H), 3.90 (s, 3H), 3.86 (s, 3H), 3.75 (s, 3H).

### Example 116

### 2-amino-6-chloro-9-((5-methoxy-4,6-dimethylpyridin-3-yl)methyl)-7-(4-methylpent-3-enyl)-7H-purin-8(9H)-one

The title compound was obtained by alkylation of 2-amino-6-chloro-9-((5-methoxy-4,6-dimethylpyridin-3-yl)methyl)-7H-purin-8(9H)-one with 1-Chloro-4-methyl-pent-3-ene according to the general procedure 1.3. HPLC Rt: 5.57 min. ¹H-NMR (CDCl₃) δ 8.31 (s, 1H), 5.12 (t, 1H), 4.98 (s, 2H), 4.87 (s, 2H), 4.00 (t, 2H), 3.71 (s, 3H), 2.47 (s, 3H), 2.42 (s, 3H), 2.42-2.38 (m, 2H), 1.63 (s, 3H), 1.52 (s, 3H).

### Example 117

### 7-(2-(1H-pyrrol-1-yl)ethyl)-2-amino-6-chloro-9-((5-methoxy-4,6-dimethylpyridin-3-yl)methyl)-7H-purin-8(9H)-one

The title compound was obtained by alkylation of 2-amino-6-chloro-9-((5-methoxy-4,6-dimethylpyridin-3-yl)methyl)-7H-purin-8(9H)-one with 1-(2-Chloro-ethyl)-1H-pyrrole according to the general procedure 1.3. HPLC Rt: 4.87 min. ¹H-NMR (CDCl₃) δ 8.31 (s, 1H), 6.56 (m, 2H), 6.08 (m, 2H), 4.95 (s, 2H), 4.93 (s, 2H), 4.35 (t, 2H), 4.21 (t, 2H), 3.72 (s, 3H), 2.49 (s, 3H), 2.38 (s, 3H).

### Example 118

### 2-amino-6-chloro-7-(2-(diisopropylamino)ethyl)-9-((5-methoxy-4,6-dimethylpyridin-3-yl)methyl)-7H-purin-8(9H)-one

The title compound was obtained by alkylation of 2-amino-9-(5-methoxy-4,6-dimethylpyridin-3-yl)methyl)-6-chloro-7H-purin-8(9H)-one with N-(2-chloroethyl)-N-isopropylpropan-2-anine according to the general procedure 1.3. HPLC Rt: 3.92 min. ¹H-NMR (CDCl₃) δ 8.30 (s, 1H), 5.30 (s, 2H), 4.98 (s, 2H), 4.02 (br t, 2H), 3.70 (s, 3H), 3.01 (m, 2H), 2.70 (br t, 2H), 2.47 (s, 3H), 2.41 (s, 3H), 0.92 (m, 12H).

### BIOLOGY EXAMPLES

### Example 119

### HER2 Degradation Assay

MCF7 breast carcinoma cells (ATCC) were grown in Dulbecco's modified Eagle's medium (DMEM) containing 10% fetal bovine serum (FBS) and 10 mM HEPES, and plated in 24 well plates (50% confluent). Twenty-four hrs later (cells are 65-70% confluent), test compounds were added and incubated overnight for 16 h. For the less potent compounds, the amounts added were 100 μM, 30 μM, 10 μM and 1 μM, and for more potent compounds, the amounts added were 1 μM, 0.3 μM, 0.1 μM, 0.03 μM, 0.01 μM and 0.003 μM. The wells were washed with 1 mL phosphate buffered saline (PBS), and 200 μL trypsin was added to each well. After trypsinization was complete, 50 μL of FBS was added to each well. Then 200 μL cells was transferred to 96 well plates. The cells were pipetted up and down to obtain a single cell suspension. The plates were centrifuged at 2,500 rpm for 1 min using a Sorvall Legend RT™0 tabletop centrifuge (Kendro Laboratory Products, Asheville, N.C.). The cells were then washed once in PBS containing 0.2% BSA and 0.2% sodium azide (BA buffer). Phycoerythrin (PE) conjugated anti HER2/Neu antibody (Becton Dickinson, #340552), or PE conjugated anti-keyhole limpet hemocyanin [KLH] (Becton Dickinson; #340761) control antibody was added at a dilution of 1:20 and 1:40 respectively (final concentration was 1 μg/mL) and the cells were pipetted up and down to form a single cell suspension, and incubated for 15 mins. The cells were washed twice with 200 μL BA buffer, and resuspended in 200 μL BA buffer, and transferred to FACSCAN tubes with an additional 250 □L BA buffer. Samples were analyzed using a FACS Calibur™ flow cytometer (Becton Dickinson, San Jose, Calif.) equipped with Argon-ion laser that emits 15 mW of 488 nm light for excitation of the PE fluorochrome. 10,000 events were collected per sample. A fluorescence histogram was generated and the mean fluorescence intensity (MFI) of each sample was determined using Cellquest software. The background was defined as the MFI generated from cells incubated with control IgG-PE, and was subtracted from each sample stained with the HER2/Neu antibody. Cells incubated with DMSO were used as untreated controls since the compounds were resuspended in DMSO. Percent degradation of HER2 was calculated as follows:
% HER2 degraded=[(MFI untreated cells−MF1 treated cells)/MFI untreated cell]×100

The HER2 degradation ability of selected compounds of the invention based on this assay is summarized in Table 2. IC50 is defined as the concentration at which there was 50% degradation of the HER2/Neu protein.

### Example 120

### MTS Assay

MTS assays measure the cytotoxicity of geldanamycin derivatives. MTS (3-(4,5-dimethylthiazol-2-yl)-5-(3-carboxymethoxyphenyl)-2-(4-sulfophenyl)-2H-tetrazolium) is a tetrazolium dye that is converted to a formazan product by dehydrogenase enzymes of metabolically active cells (Corey, A. et al. “Use of an aqueous soluble tetrazolium/formazan assay for cell growth assays in culture,” Cancer Commun. 1991, 3, 207-212). Cells were seeded in 96 well plates at 2000 cells/well and allowed to adhere overnight in Dulbecco's modified Eagle's medium supplemented with 10% fetal bovine serum. The final culture volume was 100 μl. Viable cell number was determined by using the Celltiter 96 AQueous Non-radioactive Cell Proliferation Assay (Promega, Madison Wis.). The MTS/PMS (phenazine methosulfate) solution was mixed at a ratio of 20:1, and 20 μL was added per well to 100 μl of culture medium. After 2-4 hours, the formation of the formazan product was measured at 490 nm absorbance using a multiwell plate spectrophotometer. Background was determined by measuring the Abs 490 nm of cell culture medium and MTS-PMS in the absence of cells and was subtracted from all values. Percent viable cells was calculated as follows:
% viable cells=(Abs at 490 nm treated cells/Abs at 490 nm untreated cells)×100

The effect of selected compounds of the invention on MCF7 breast carcinoma cells according to the MTS assay is summarized in Table 2. IC50 was defined as the concentration of the compound which gave rise to 50% reduction in viable cell number.

TABLE 2
Biological Activities of Selected Compounds of the Invention

| | | | HER2 | MTS |
|---|---|---|---|---|
| | Ex | | IC₅₀ | IC₅₀ |
| S.No | # | Structure | (μM) | (μM) |
| 90 | 98 | 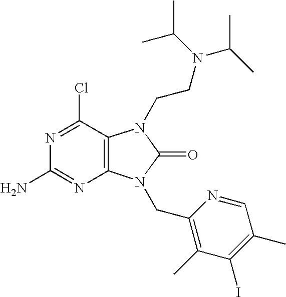 | 30 | 600 |
| 87 | 96 | 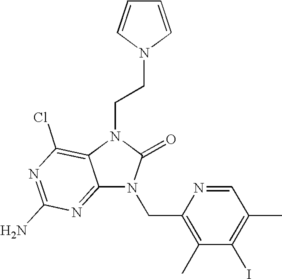 | 42 | 300 |
| 88 | 6 | 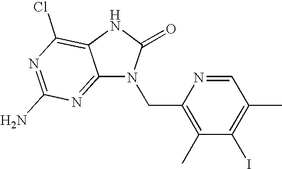 | 70 | 300 |
| 95 | 105 | 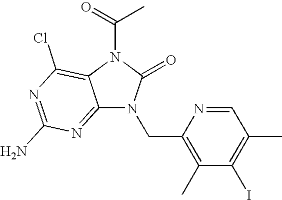 | 90 | 300 |
| 78 | 89 | 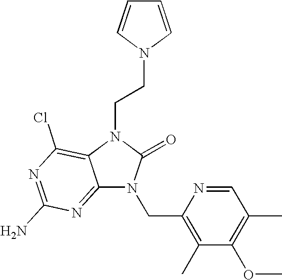 | 120 | 1000 |
| 91 | 99 | 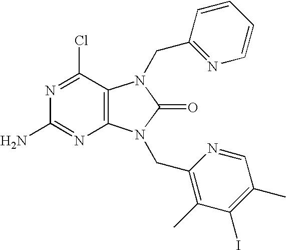 | 120 | >1000 |
| 97 | 107 | 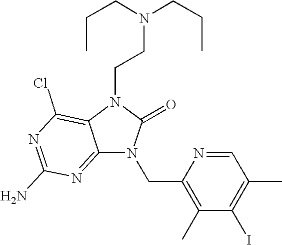 | 130 | >1000 |
| 9 | 20 | 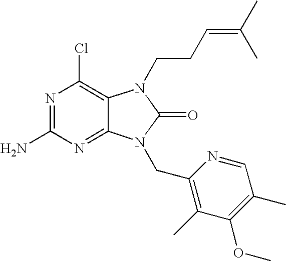 | 140 | >1000 |
| 98 | 108 | 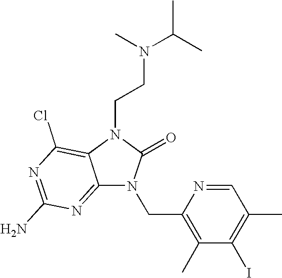 | 140 | 700 |
| 24 | 35 | 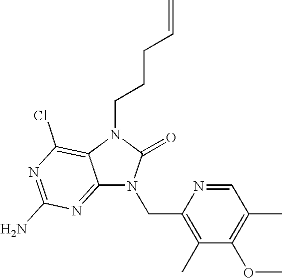 | 160 | 1000 |
| 60 | 71 | 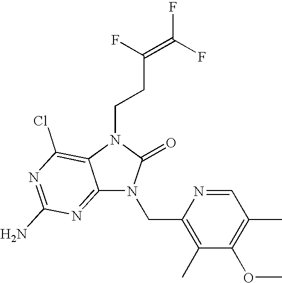 | 160 | >1000 |
| 69 | 80 | 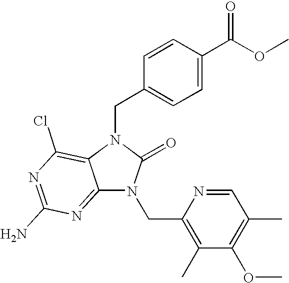 | 160 | >1000 |
| 71 | 82 | 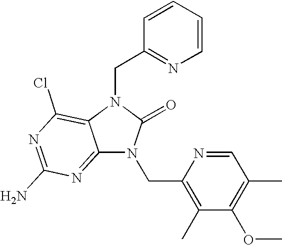 | 170 | 1000 |

The foregoing examples are not limiting and are merely illustrative of various aspects and embodiments of the present invention. All documents cited herein are indicative of the levels of skill in the art to which the invention pertains and are incorporated by reference herein in their entireties. None, however, is admitted to be prior art.

One skilled in the art will readily appreciate that the present invention is well adapted to carry out the objects and obtain the ends and advantages mentioned, as well as those inherent therein. The methods and compositions described illustrate preferred embodiments, are exemplary, and are not intended as limitations on the scope of the invention. Certain modifications and other uses will occur to those skilled in the art, and are encompassed within the spirit of the invention, as defined by the scope of the claims.

The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described, or portions thereof. It is recognized that various modifications are possible within the scope of the invention claimed. Thus, it should be understood that although the present invention has been specifically disclosed by preferred embodiments, optional features, modifications and variations of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, e.g., genuses, those skilled in the art will recognize that the invention is also thereby described in terms of any individual member or subgroup of members of the Markush group or subgenus, and exclusions of individual members as appropriate, e.g., by proviso.

What is claimed is:

1. A compound represented by Formula I, or a polymorph, solvate, tautomer, enantiomer, pharmaceutically acceptable salt or prodrug thereof: 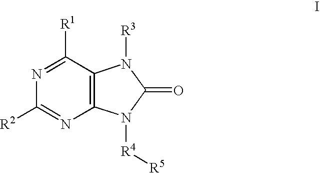
wherein:
R¹ is halogen, —OR⁸, —SR⁸, or lower alkyl;
R² is —NR⁹R¹⁰;
R³ is selected from the group consisting of hydrogen, —C(O)OH, —C(O)R⁹, —CH₂CN, alkyl, substituted alkyl, alkenyl, substituted alkenyl, alkynyl, substituted alkynyl, perhaloalkyl, substituted perhaloalkyl, alkoxyalkylene, substituted alkoxyalkylene, perhaloalkoxy, perhaloacyl, benzyl, substituted benzyl, phenethyl, substituted phenethyl, —(CH₂)ₘ-alicyclyl, —(CH₂)ₘ-substituted alicyclyl, —(CH₂)ₘ-aryl, —(CH₂)ₘ-substituted aryl, —(CH₂)ₘ-heterocyclyl, —(CH₂)ₘ-substituted heterocyclyl, —(CH₂)ₘ-heteroaryl, —(CH₂)ₘ-substituted heteroaryl and —(CH₂)—NR⁸R¹⁰; wherein
m=1, 2 or 3;
the substituents are selected from the group comprising hydrogen, halogen, lower alkyl, lower alkenyl, lower alkynyl, —CN, —C(O)OH, —NO₂, —OR⁸, —SR⁸, —C(O)R⁹, —NR⁸R¹⁰, lower aryl, heteroaryl, alicyclyl, lower heterocyclyl, araalkyl, heteroaraalkyl, amino, alkylamino, dialkylamino, diarylalkylamino, perhaloalkyl, perhaloalkoxy, perhaloacyl, guanidyl, pyridinyl, thiophenyl, furanyl, indolyl, indazolyl, phosphonates, phosphatyl, phosphoramidyl, sulfanyl, sulfinyl, sulfonyl, sulfonamidyl, carbamyl, uryl, thiouryl and thioamidyl;
R⁴ is —C(O)—, —C(S)—, —S(O)₂—, —S(O)₂N— or —(CH₂)ₙ—, wherein n=0, 1, 2 or 3;
R⁵ is alkyl, alicyclic, heterocyclic, aryl or heteroaryl; all optionally substituted with hydrogen, halogen, lower alkyl, lower alkenyl, lower alkynyl, lower aryl, lower heteroaryl, lower heterocyclic, lower alicyclic, aralkyl, aryloxyalkylene, alkoxyalkylene, perhaloalkyl, perhaloalkyloxy, perhaloacyl, —CN, —N₃, —NO₂, —SR⁸, —OR⁸, —C(O)R⁹ or —NR⁸R¹⁰;
R⁹ is lower alkyl, lower alkenyl, lower alkynyl, lower heterocyclic, lower aryl, lower heteroaryl, —NR⁹R¹⁰ or —OR¹¹;
R⁸ is hydrogen, lower alkyl, lower alkenyl, lower alkynyl, lower heterocyclic, lower aryl or —C(O)R⁹;
R¹⁰ is hydrogen, lower alkyl, lower alkenyl, lower alkynyl, lower heterocyclic or lower aryl; or
R⁸ and R¹⁰ are taken together with the N atom to which they are attached to form a ring comprising 3-7 ring atoms, wherein, in addition to the ring N atom, optionally 1-3 of the ring atoms are heteroatoms selected from the group O, S and N;
R¹¹ is lower alkyl, lower alkenyl, lower alkynyl or lower aryl;
with the provisos that if n=0, then R⁵ cannot be heterocyclyl; and
if R¹ is Cl or OH and R² is NH₂, then R³ cannot be H or allyl.

2. The compound of claim 1, or a polymorph, solvate, tautomer, enantiomer, pharmaceutically acceptable salt or prodrug thereof, wherein any of said aryl, heteroaryl, alicyclic or heterocyclic groups are monocyclic or bicyclic.

3. The compound of claim 1 or a polymorph, solvate, tautomer, enantiomer, pharmaceutically acceptable salt or prodrug thereof, wherein:
R¹ is halogen;
R² is —NHR⁸, and
R⁸ is hydrogen or —C(O)R⁹.

4. The compound of claim 1, or a polymorph, solvate, tautomer, enantiomer pharmaceutically acceptable salt or prodrug thereof, wherein:
R¹ is chloro or bromo;
R² is —NHR⁸;
R⁸ is hydrogen or —C(O)R⁹; and
R³ is hydrogen, lower alkyl, lower alkenyl, lower alkynyl, lower perhaloalkyl, lower aryl, lower heteroaryl or —C(O)R⁹.

5. The compound of claim 1, or a polymorph, solvate, tautomer, enantiomer, pharmaceutically acceptable salt or prodrug thereof, wherein:
R² is —NHR⁸;
R⁸ is hydrogen or —C(O)R⁹; and
R⁴ is —CH₂—.

6. The compound of claim 1, or a polymorph, solvate, tautomer, enantiomer, pharmaceutically acceptable salt or prodrug thereof, wherein:
R¹ is halogen;
R² is —NH₂; and
R⁴ is —CH₂—;

7. The compound of claim 6, or a polymorph, solvate, tautomer, enantiomer, pharmaceutically acceptable salt or prodrug thereof, wherein
R¹ is chloro or bromo; and
R⁵ is phenyl having at least three substituents.

8. The compound of claim 6, or a polymorph, solvate, tautomer, enantiomer, pharmaceutically acceptable salt or prodrug thereof, wherein
R¹ is chloro or bromo; and
R⁵ is a pyridyl having at least two substituents.

9. The compound of claim 6, or a polymorph, solvate, tautomer, enantiomer, pharmaceutically acceptable salt or prodrug thereof, wherein
R¹ is chloro or bromo; and
R⁵ is 1-oxy-pyridyl having at least two substituents.

10. The compound of claim 6, wherein said compound is a member selected from the group below, or a polymorph, solvate, ester, tautomer, enantiomer, pharmaceutically acceptable salt or prodrug thereof:

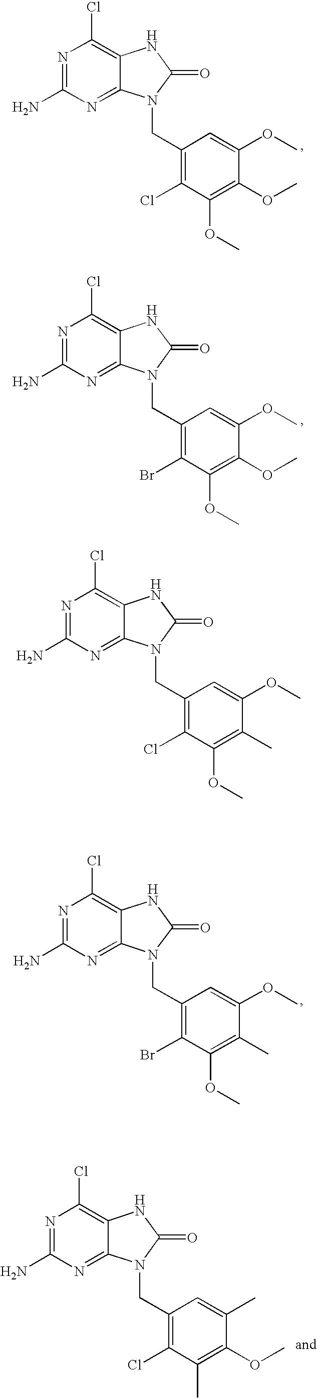

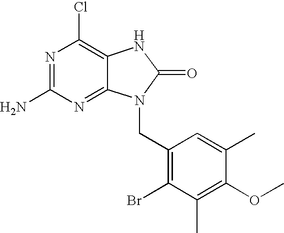

11. The compound of claim 6, wherein said compound is a member selected from the group below, or a polymorph, solvate, ester, tautomer, enantiomer, pharmaceutically acceptable salt or prodrug thereof:

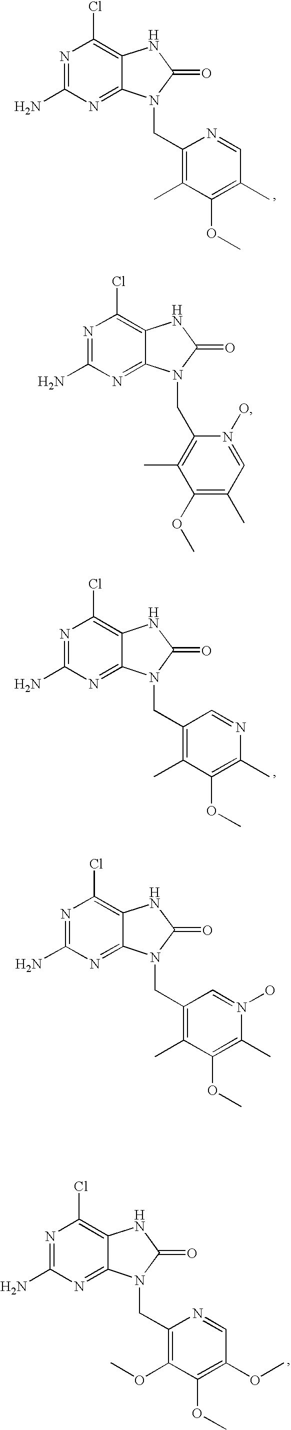

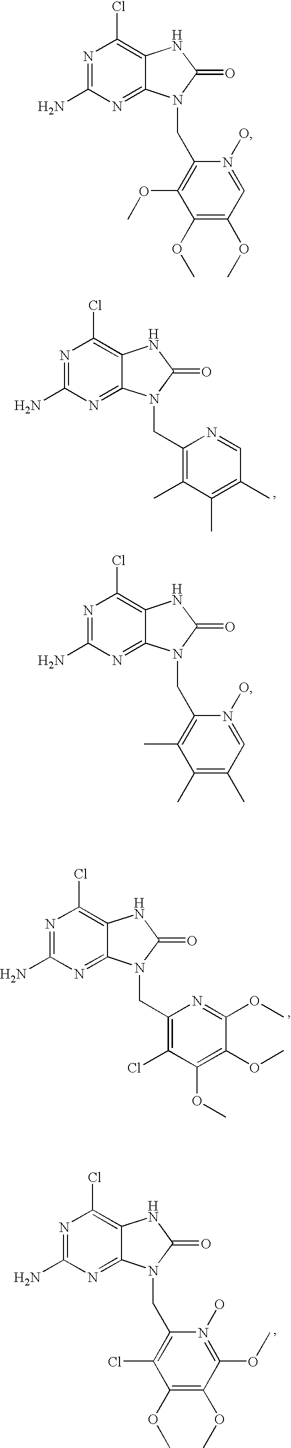

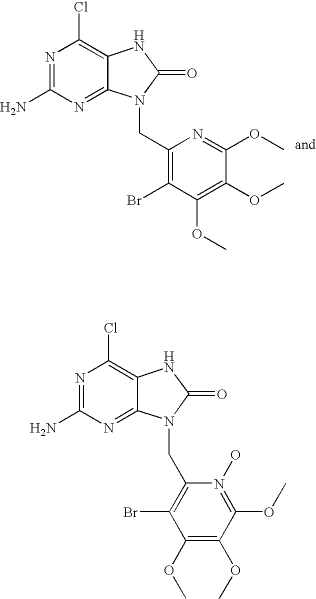

12. The compound of claim 6, wherein said compound is a member selected from the group below, or a polymorph, solvate, ester, tautomer, enantiomer, pharmaceutically acceptable salt or prodrug thereof:

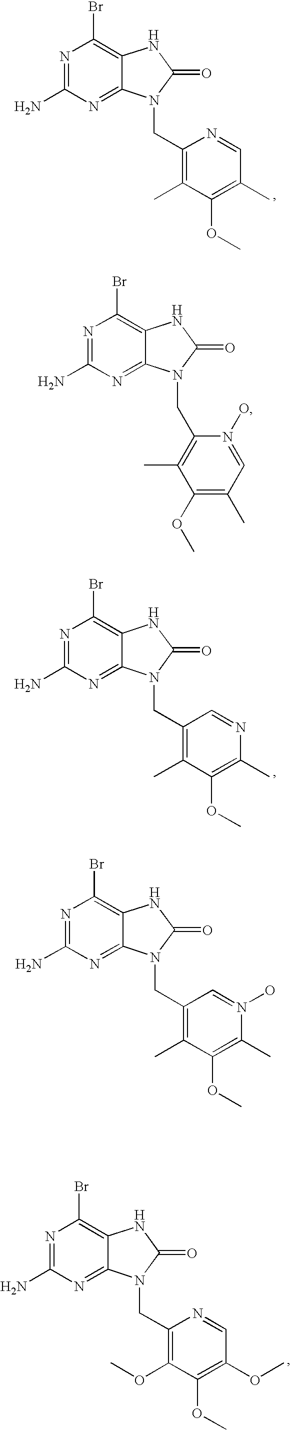

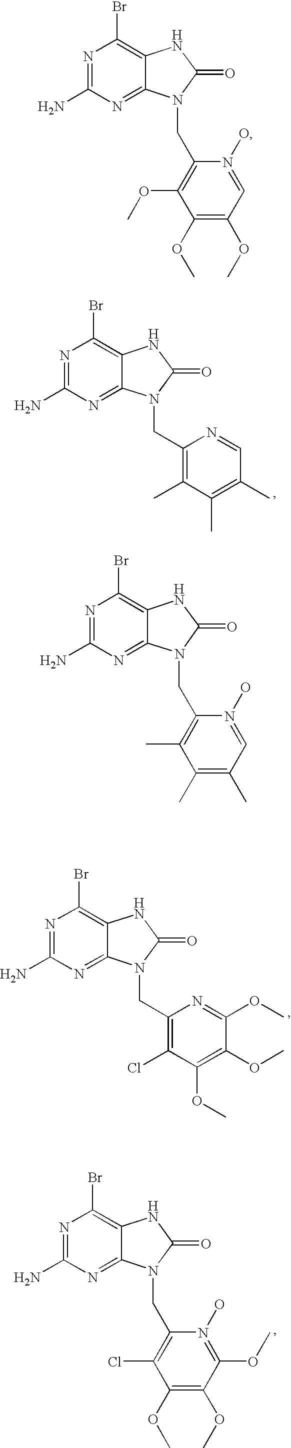

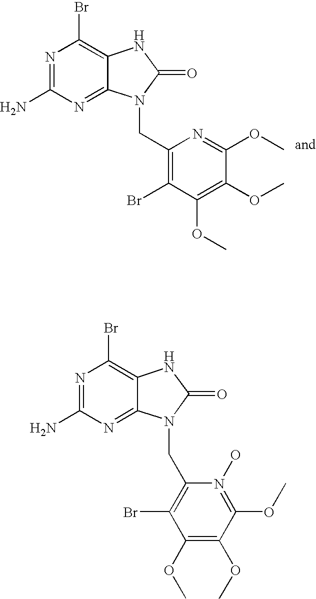

13. The compound of claim 6, wherein said compound is a member selected from the group below, or a polymorph, solvate, ester, tautomer, enantiomer, pharmaceutically acceptable salt or prodrug thereof:

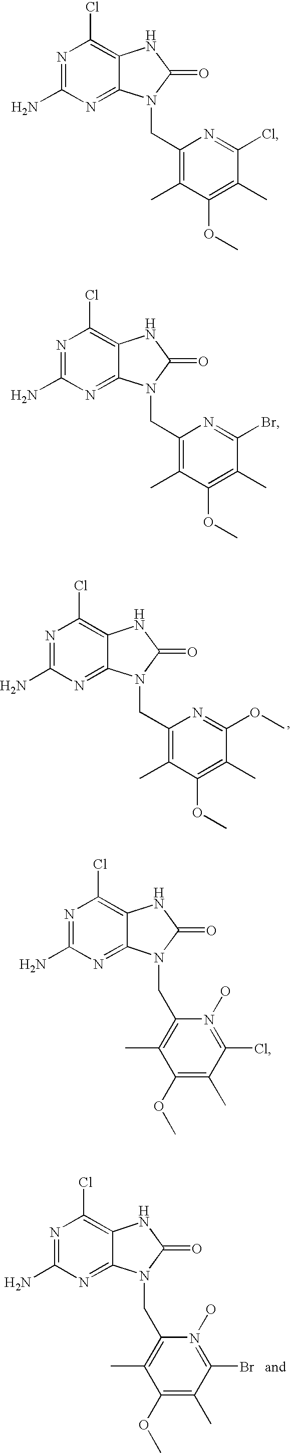

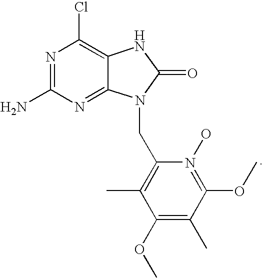

14. The compound of claim 6, wherein said compound is a member selected from the group below, or a polymorph, solvate, ester, tautomer, enantiomer, pharmaceutically acceptable salt or prodrug thereof:

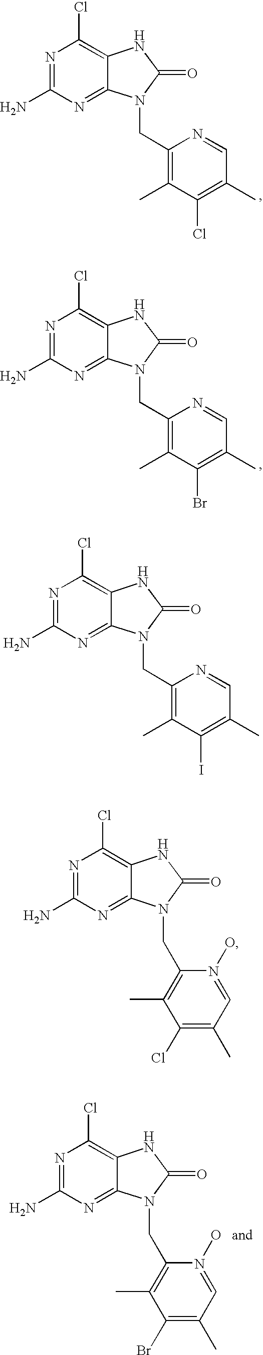

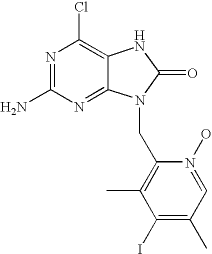

15. The compound of claim 6, wherein said compound is a member selected from the group below, or a polymorph, solvate, ester, tautomer, enantiomer, pharmaceutically acceptable salt or prodrug thereof:

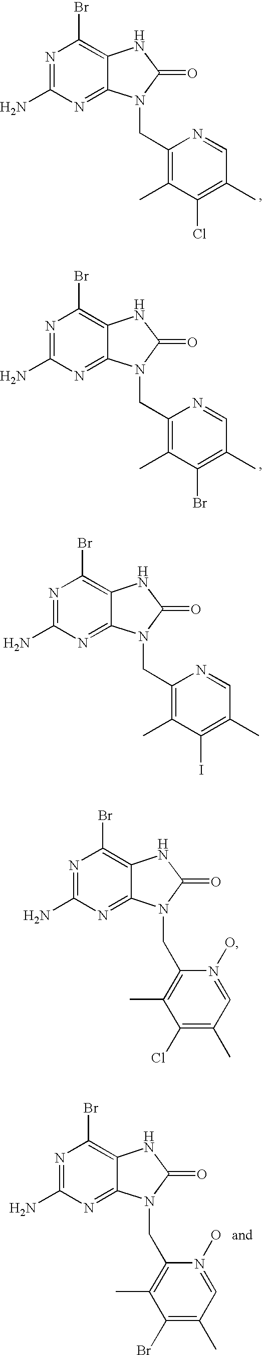

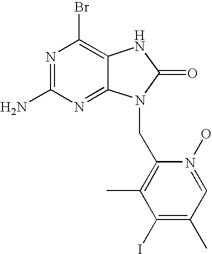

16. The compound of claim 6, wherein said compound is a member selected from the group below, or a polymorph, solvate, ester, tautomer, enantiomer, pharmaceutically acceptable salt or prodrug thereof:

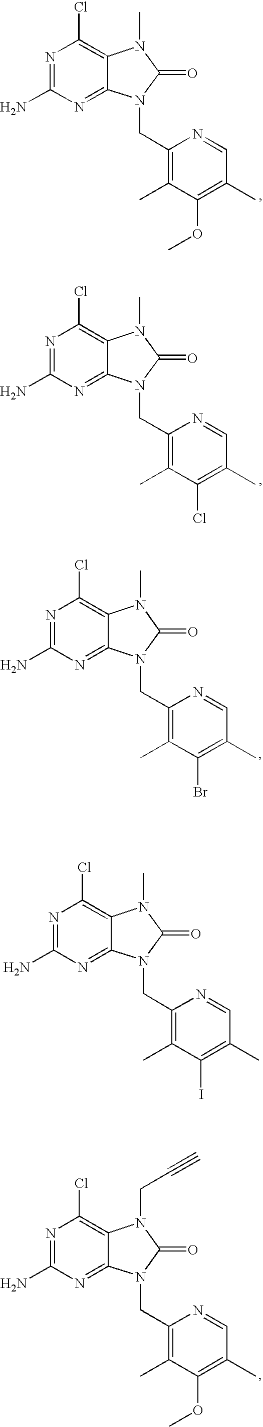

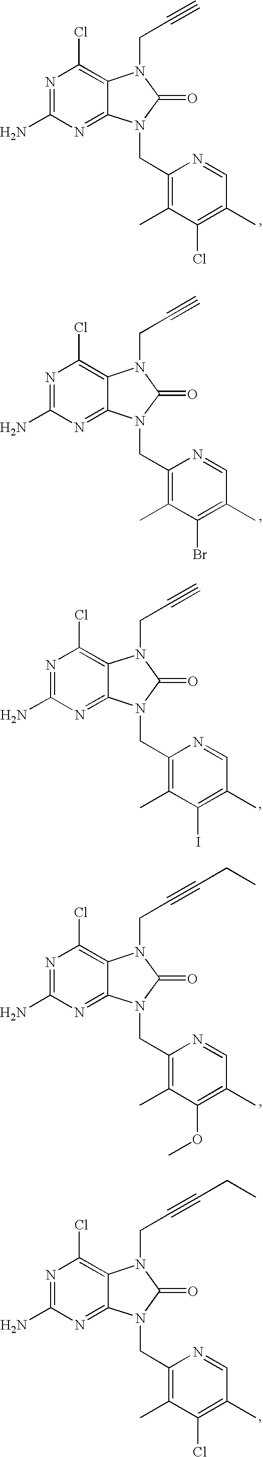

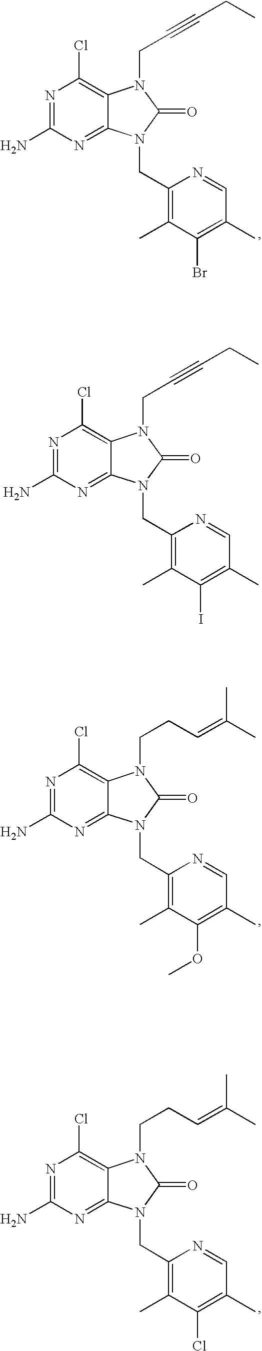

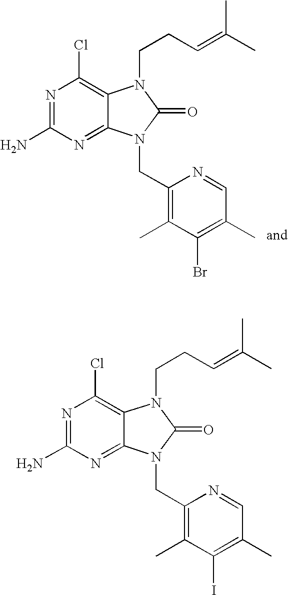

17. The compound of claim 6, wherein said compound is a member selected from the group below, or a polymorph, solvate, ester, tautomer, enantiomer, pharmaceutically acceptable salt or prodrug thereof:

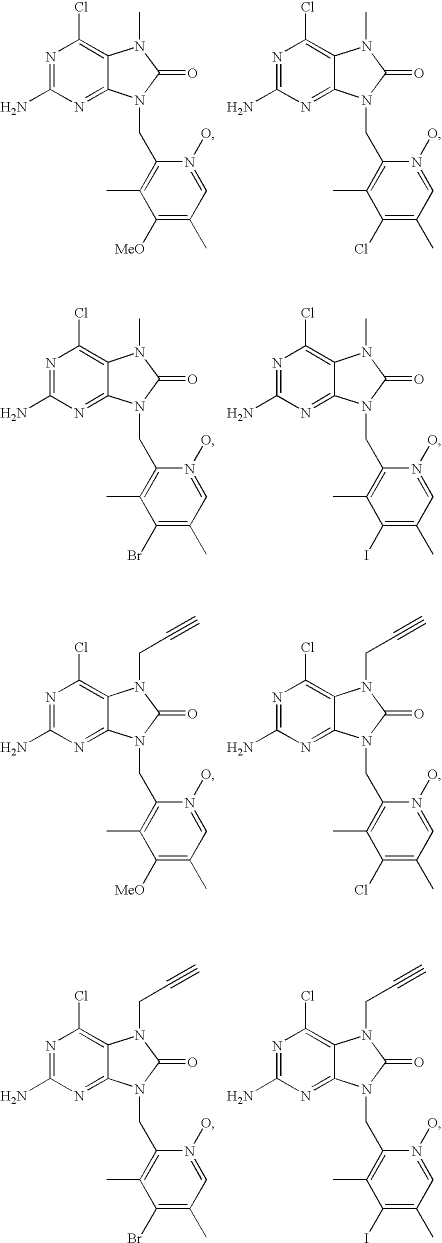

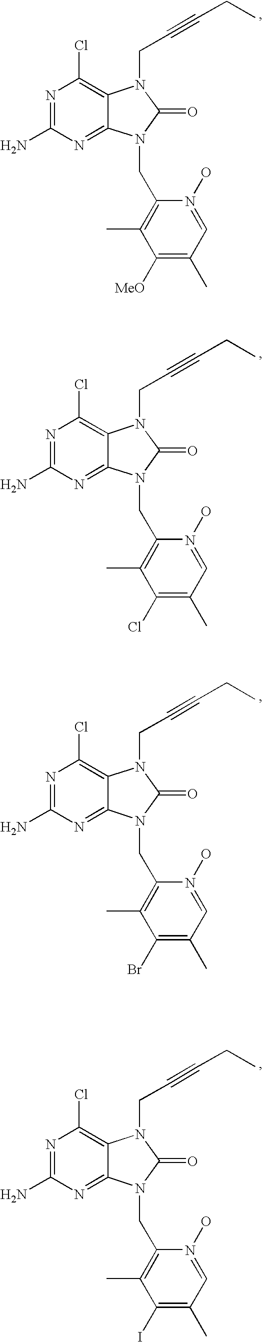

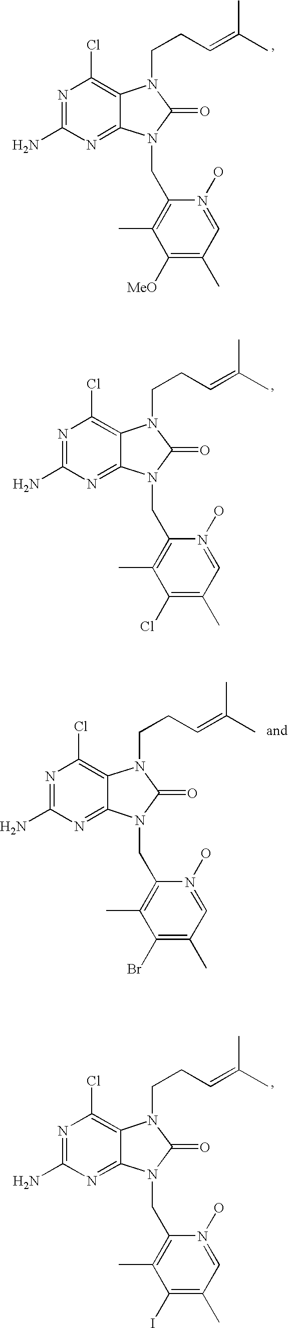

18. The compound of claim 6, wherein said compound is a member selected from the group below, or a polymorph, solvate, ester, tautomer, enantiomer, pharmaceutically acceptable salt or prodrug thereof:

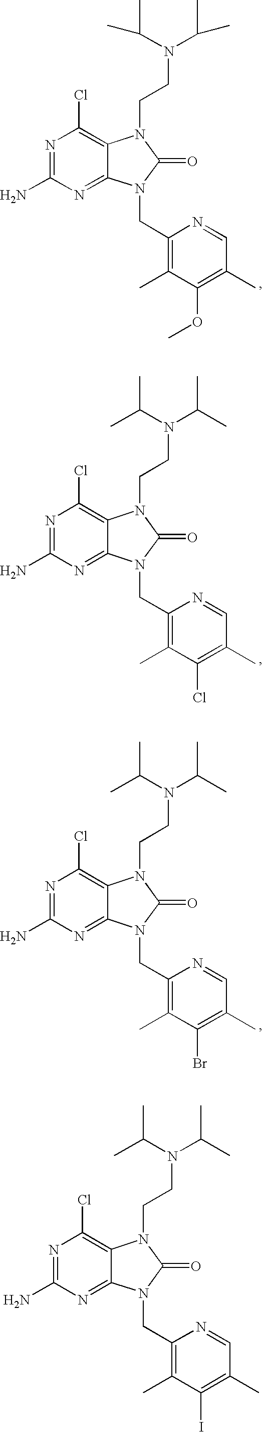

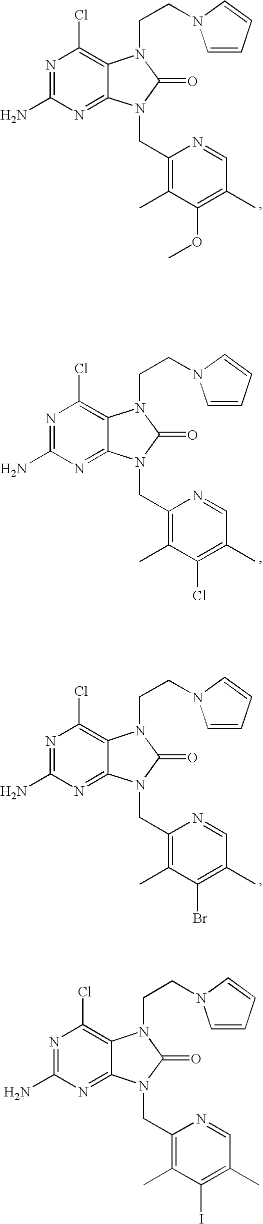

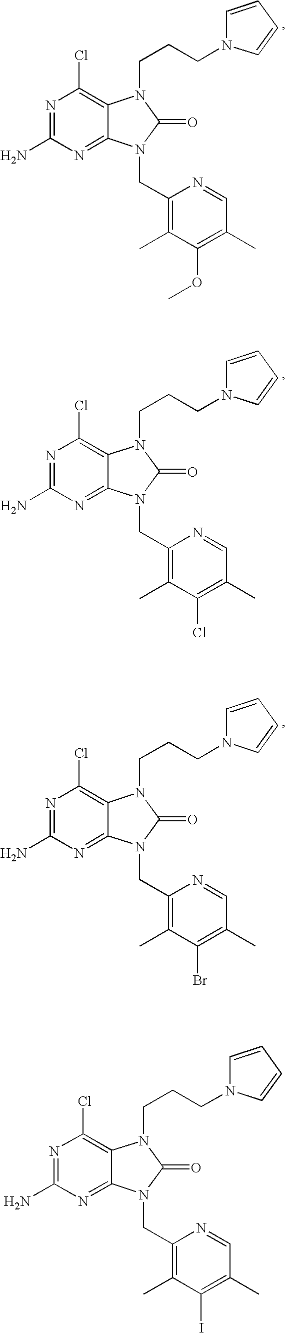

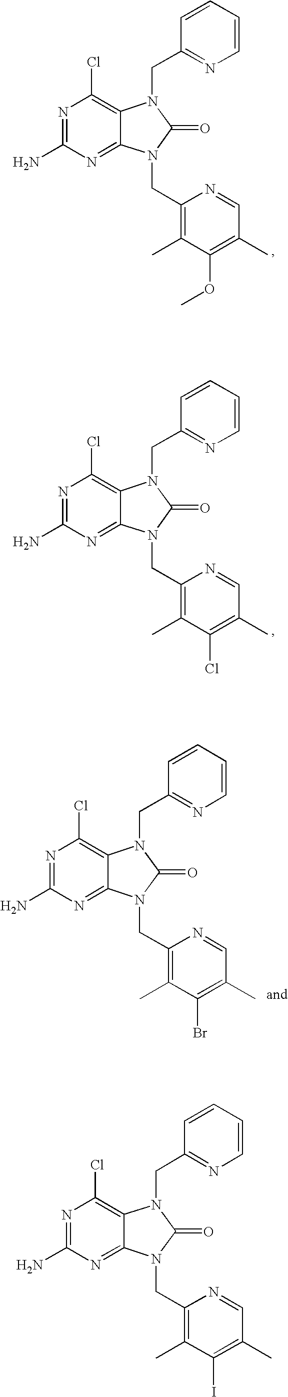

19. The compound of claim 6, wherein said compound is a member selected from the group below, or a polymorph, solvate, ester, tautomer, enantiomer, pharmaceutically acceptable salt or prodrug thereof:

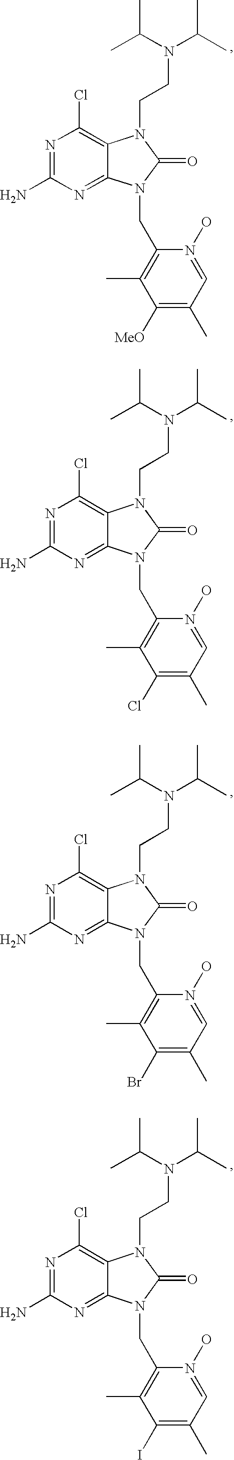

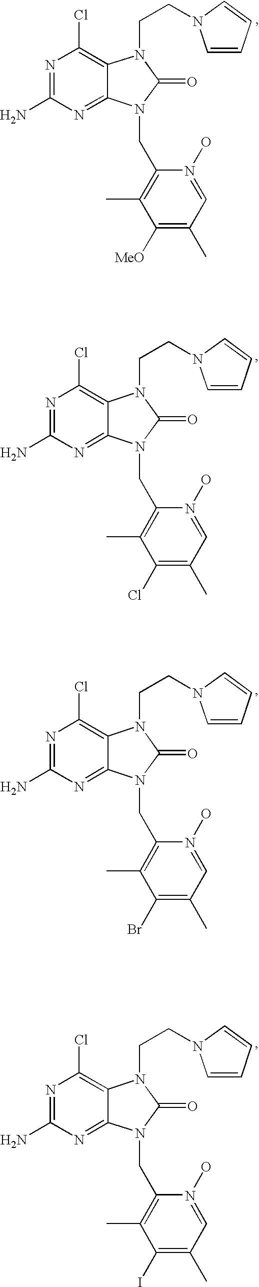

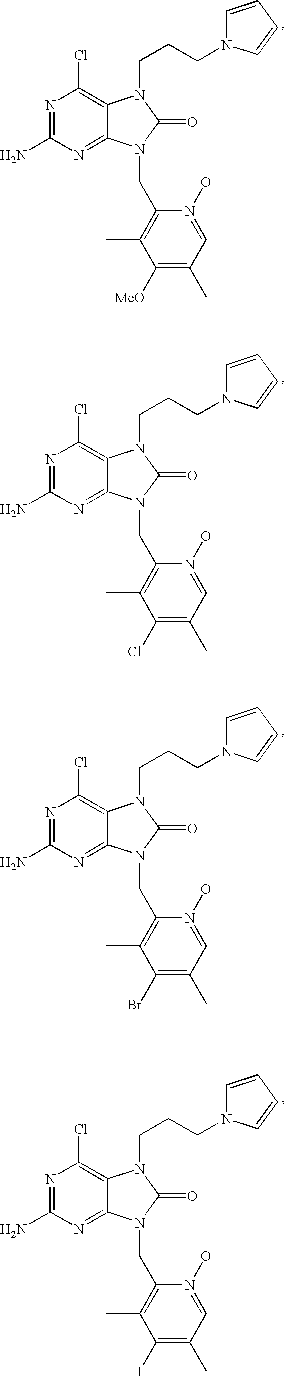

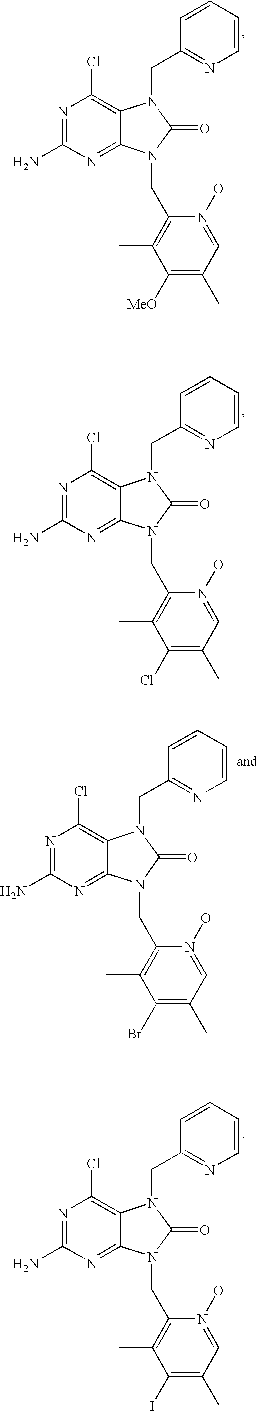

20. The compound of claim 6, wherein said compound is a member selected from the group below, or a polymorph, solvate, ester, tautomer, enantiomer, pharmaceutically acceptable salt or prodrug thereof:

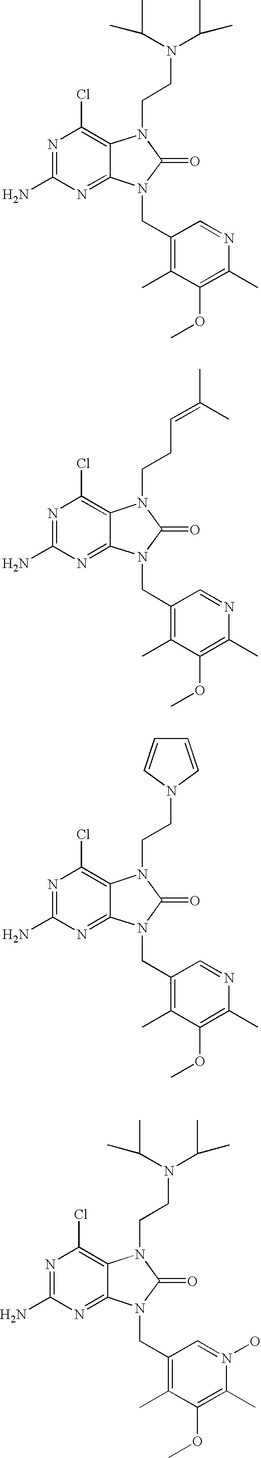

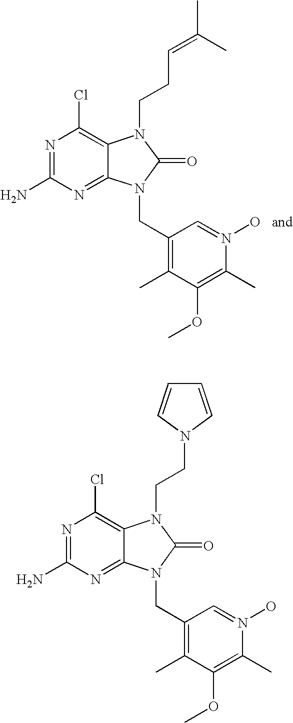

21. The compound of claim 6, or a polymorph, solvate, ester, tautomer, enantiomer, pharmaceutically acceptable salt or prodrug thereof, with the following formula:

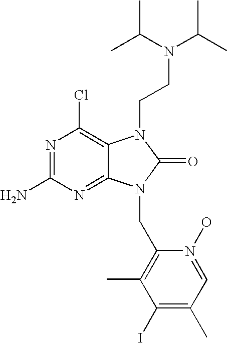

22. The compound of claim 6, or a polymorph, solvate, ester, tautomer, enantiomer, pharmaceutically acceptable salt or prodrug thereof, with the following formula:

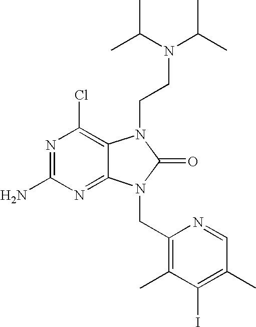

23. The compound of claim 6, or a polymorph, solvate, ester, tautomer, enantiomer, pharmaceutically acceptable salt or prodrug thereof, with the following formula:

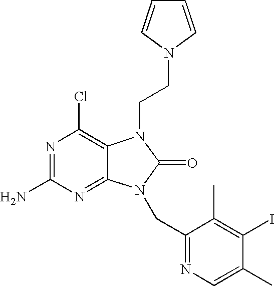

24. The compound of claim 6, or a polymorph, solvate, ester, tautomer, enantiomer, pharmaceutically acceptable salt or prodrug thereof, with the following formula:

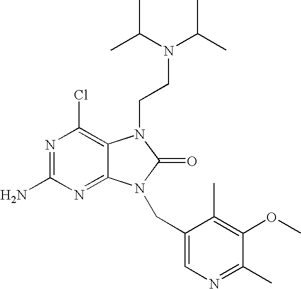

25. The compound of claim 6, or a polymorph, solvate, ester, tautomer, enantiomer, pharmaceutically acceptable salt or prodrug thereof, with the following formula:

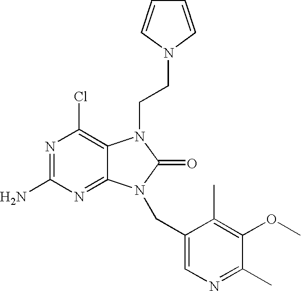

26. The compound of claim 6, or a polymorph, solvate, ester, tautomer, enantiomer, pharmaceutically acceptable salt or prodrug thereof, with the following formula:

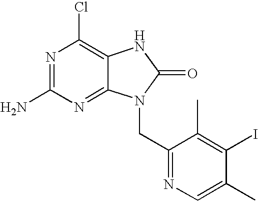

27. The compound of claim 6, wherein said compound is represented by the formula below, or a polymorph, solvate, ester, tautomer, enantiomer, pharmaceutically acceptable salt or prodrug thereof:

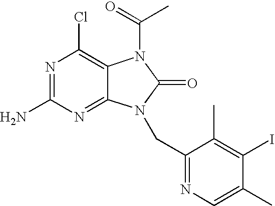

28. The compound of claim 6, wherein said compound is represented by the formula below, or a polymorph, solvate, ester, tautomer, enantiomer, pharmaceutically acceptable salt or prodrug thereof:

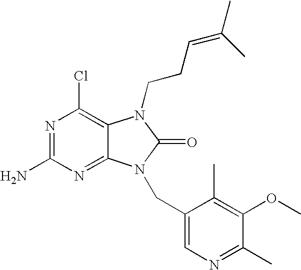

29. The compound of claim 6, wherein said compound is represented by the formula below, or a polymorph, solvate, ester, tautomer, enantiomer, pharmaceutically acceptable salt or prodrug thereof:

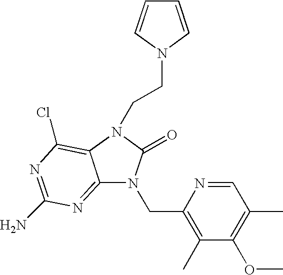

30. The compound of claim 6, wherein said compound is represented by the formula below, or a polymorph, solvate, ester, tautomer, enantiomer, pharmaceutically acceptable salt or prodrug thereof:

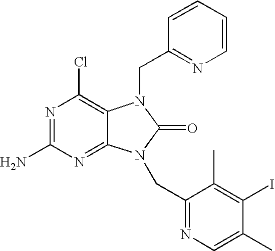

31. The compound of claim 6, wherein said compound is represented by the formula below, or a polymorph, solvate, ester, tautomer, enantiomer, pharmaceutically acceptable salt or prodrug thereof:

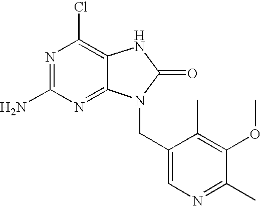

32. The compound of claim 6, wherein said compound is represented by the formula below, or a polymorph, solvate, ester, tautomer, enantiomer, pharmaceutically acceptable salt or prodrug thereof:

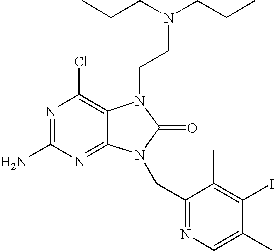

33. The compound of claim 6, wherein said compound is represented by the formula below, or a polymorph, solvate, ester, tautomer, enantiomer, pharmaceutically acceptable salt or prodrug thereof:

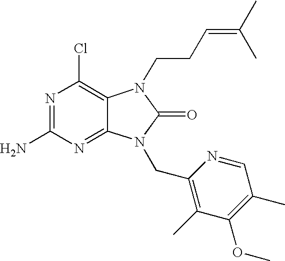

34. The compound of claim 6, wherein said compound is represented by the formula below, or a polymorph, solvate, ester, tautomer, enantiomer, pharmaceutically acceptable salt or prodrug thereof:

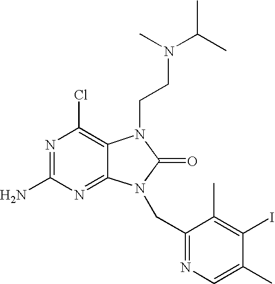

35. The compound of claim 6, wherein said compound is represented by the formula below, or a polymorph, solvate, ester, tautomer, enantiomer, pharmaceutically acceptable salt or prodrug thereof:

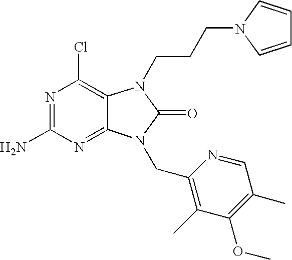

36. The compound of claim 6, wherein said compound is represented by the formula below, or a polymorph, solvate, ester, tautomer, enantiomer, pharmaceutically acceptable salt or prodrug thereof:

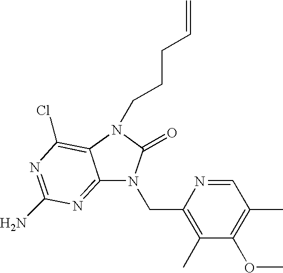

37. The compound of claim 6, wherein said compound is represented by the formula below, or a polymorph, solvate, ester, tautomer, enantiomer, pharmaceutically acceptable salt or prodrug thereof:

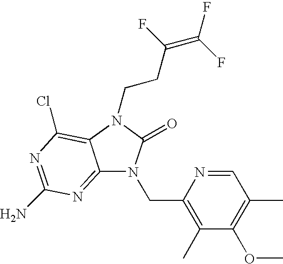

38. The compound of claim 6, wherein said compound is represented by the formula below, or a polymorph, solvate, ester, tautomer, enantiomer, pharmaceutically acceptable salt or prodrug thereof:

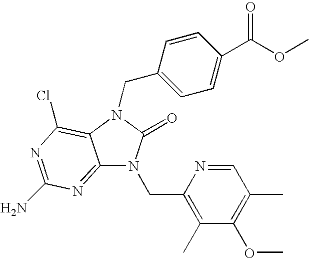

39. The compound of claim 6, wherein said compound is represented by the formula below, or a polymorph, solvate, ester, tautomer, enantiomer, pharmaceutically acceptable salt or prodrug thereof:

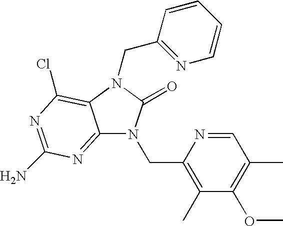

40. The compound of claim 6, wherein said compound is represented by the formula below, or a polymorph, solvate, ester, tautomer, enantiomer, pharmaceutically acceptable salt or prodrug thereof:

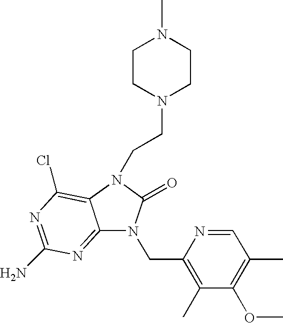

41. The compound, polymorph, solvate, tautomer, enantiomer, pharmaceutically acceptable salt or prodrug of claim 1 having from 0 to 25 substitutions, collectively.

42. A pharmaceutical composition comprising the compound, polymorph, solvate, ester, tautomer, enantiomer, pharmaceutically acceptable salt or prodrug of claim 1 and one or more pharmaceutical carriers or excipients.

43. A complex comprising the compound, polymorph, solvate, ester, tautomer, enantiomer, pharmaceutically acceptable salt or prodrug of claim 1, and at least one other compound.

44. The complex of claim 43 wherein one of said at least one other compound is an HSP90.

45. The complex of claim 44 wherein said HSP90 is human.

46. A method of inhibiting an HSP90, comprising:
contacting a cell having an HSP90 with a compound, polymorph, solvate, tautomer, enantiomer, pharmaceutically acceptable salt or prodrug of claim 1.

47. The method of claim 46 wherein said cell is a mammalian cell.

48. The method of claim 46 wherein said mammalian cell is human.

49. The method of claim 46 wherein said contacting occurs in vitro, in vivo or in situ.

50. The method of claim 46 wherein said contacting is part of an ex vivo procedure.

51. The method of claim 46 wherein said contacting is accomplished by intravenous, parenteral, oral or topical administration to a subject.

52. The method of claim 46 wherein said contacting is part of a therapy directed against cancer cells.

53. The method of claim 52 wherein said cancer cells are selected from breast cancer cells and melanoma cells.

54. Use of the compound, polymorph, solvate, ester, tautomer, enantiomer, pharmaceutically acceptable salt or prodrug of claim 1 in a chemotherapy regimen.

55. The use of claim 54 wherein said regimen is part of a combinational therapy that makes use of one or more other agents selected from the group consisting of radioisotopes, antibodies, recombinant products, small molecules, antineoplastic agents, Herceptin, taxol, taxanes and taxane derivatives, gleevac, alkylating agents, anti-metabolites; epidophyllotoxin; an antineoplastic enzyme; a topoisomerase inhibitor; procarbazine; mitoxantrone; platinum coordination complexes; biological response modifiers/growth inhibitors; hormonal/anti-hormonal therapeutic agents and haematopoietic growth factors, anthracycline drugs, vinca drugs, mitomycins, bleomycins, cytotoxic nucleosides, tepothilones, discodermolide, pteridine drugs, diynenes, podophyllotoxins, caminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin, podo-phyllotoxin derivatives, etoposide, etoposide phosphate or teniposide, melphalan, vinblastine, vincristine, leurosidine, vindesine, leurosine, paclitaxel, estramustine, carboplatin, cyclophosphamide, bleomycin, gemcitabine, ifosfamide, melphalan, hexamethyl melamine, thiotepa, cytarabine, edatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

56. Use of the compound, polymorph, solvate, tautomer, enantiomer, pharmaceutically acceptable salt or prodrug of claim 1 for treating one or more of inflammation, infectious disease, autoimmune disease, and ischemia.
